(19) United States
(12) Patent Application Publication
(10) Pub. No.: US 2025/0241927 A1
(43) Pub. Date: Jul. 31, 2025

(54) MEDICAMENT COMPRISING A P2X4 RECEPTOR ANTAGONIST FOR PREVENTING OR TREATING NOCICEPTIVE PAIN AND/OR VISCERAL PAIN

(71) Applicant: NIPPON CHEMIPHAR CO., LTD., Tokyo (JP)

(72) Inventors: Luca Antonioli, Pisa (IT); Matteo Fornai, Pisa (IT); Carolina Pellegrini, Pisa (IT); Lorenzo Di Cesare Mannelli, Firenze (IT); Carla Ghelardini, Firenze (IT); Elena Lucarini, Firenze (IT)

(21) Appl. No.: 18/694,663

(22) PCT Filed: Sep. 29, 2022
(86) PCT No.: PCT/EP2022/077138

§ 371 Date: Mar. 22, 2024

(30) Foreign Application Priority Data
Sep. 30, 2021 (IT) 102021000025124

(51) Int. Cl. A61K 31/5513; A61P 29/00

(52) U.S. Cl. CPC A61K 31/5513; A61P 29/00

(57) ABSTRACT

The present invention pertains to a medicament for preventing or treating nociceptive pain and/or visceral pain, the medicament comprising as an active ingredient a compound having a P2X4 receptor antagonistic action, or a pharmaceutically acceptable salt thereof.

### TECHNICAL FIELD

The present invention relates to a prevention or treating agent for nociceptive pain and/or visceral pain.

### BACKGROUND ART

According to the International Association for the Study of Pain, pain is defined as “an unpleasant sensory and emotional experience associated with, or resembling that associated with, actual or potential tissue damage”. Pain is classified by the cause, the site of occurrence, and the like. For example, pain occurring through a nociceptor is classified as “nociceptive pain” and generally distinguished from “neuropathic pain” which does not occur through a nociceptor. Nociceptive pain is generally classified into somatic pain and visceral pain, depending on the site of the pain. As defined by the International Association for the Study of Pain, pain is an unpleasant sensory or emotional experience, and control thereof is important in improving QOL.

For example, irritable bowel syndrome or inflammatory bowel disease is known as a cause of visceral pain.

Irritable bowel syndrome (IBS) is a typical functional gastrointestinal disorder and is a chronic disorder of which core symptoms include abdominal pains, abdominal discomfort, and an altered bowel habit related thereto, such as diarrhea and constipation. The symptom of the irritable bowel syndrome may be exacerbated by a psychological factor or stress.

As a drug treatment for the irritable bowel syndrome, for example, a tricyclic antidepressant is effective for a diarrhea type, however, a side effect such as drowsiness, constipation, or dry mouth is likely to be caused. A 5-HT₃ antagonist has been used as a drug for constipation-predominant-type irritable bowel syndrome overseas. In addition, a drug having a 5-HT₄ receptor stimulant action or 5-HT₄ receptor stimulant and 5-HT₃ receptor stimulant actions has been effective for abdominal pains and constipation overseas. However, 5-HT drugs are limitedly used due to side effects. Therefore, drug therapy for the irritable bowel syndrome has been so far extremely limited (Non Patent Literature 1).

Inflammatory bowel disease (IBD) is a multifactorial immune disorder which causes a chronic, recurrent inflammation in the intestinal tract. The inflammatory bowel disease is classified broadly into Crohn's disease (CD) and ulcerative colitis (UC) and may be referred to as narrowly defined inflammatory bowel disease such as a simple ulcer and non-specific multiple ulcers of the small intestine (Non Patent Literature 2).

Crohn's disease and ulcerative colitis clinically share common symptoms of diarrhea, a bloody stool, abdominal pains, and the like, however, etiology of the inflammatory bowel disease is currently unclear.

Examples of a cause of increase in incidence of the inflammatory bowel disease can include insufficient effectiveness and/or safety of a conventional drug (corticosteroid, immunosuppressant, and salicylate). Examples of other treatment options can include treatment using a biotechnological drug, and even though the treatment exhibits a certain degree of effectiveness, the treatment is accompanied by a serious side effect.

Therefore, there is currently an ongoing effort to search for a novel drug from which a positive benefit/risk profile is obtained, instead of settling for a pharmacological treatment of IBD (Non Patent Literature 3).

Under such circumstance, over many years, many evidence that ATP plays an important role regarding generation of an immune/inflammatory response have been reported. From these reports, it has been revealed that a P2X4 receptor is involved in mediating such ATP-regulated activation. Particularly, the P2X4 receptor maintains a proinflammatory response which sustains release of a proinflammatory cytokine such as IL-1β and IL-18. Furthermore, there are several evidence showing that P2X4 is involved in a plurality of immune/inflammatory pathological conditions such as postischemic inflammation, rheumatoid arthritis, respiratory tract inflammation of asthma, a neurodegenerative disease, and metabolic syndrome (Non Patent Literatures 4 to 6).

As described above, the conventional drugs or treatments in Non Patent Literatures 1 to 6 can also be used as medicaments for treating the irritable bowel syndrome or the inflammatory bowel disease, however, it is known that effectiveness thereof is weak, or the drugs or treatments can cause a certain side effect, and thus the drugs are not effective as the medicament for treating the irritable bowel syndrome or the inflammatory bowel disease.

Patent Literature 1 discloses a P2X4 receptor antagonist.

However, a compound described in an example in Patent Literature 1 is a selective serotonin reuptake inhibitor, for example, Paroxetine, Fluoxetine, or the like, and has a structure that is completely different from that of a compound of the present application, which is a benzodiazepine derivative compound. Furthermore, only a result of an experiment using a neuropathic pain pathology model in which a nerve injury (L5 spinal nerve injury model) is performed is shown, and whether the P2X4 receptor antagonist has a treatment effect in the irritable bowel syndrome or the inflammatory bowel disease is not determined.

In addition, Patent Literature 2 also discloses a compound exhibiting a P2X4 receptor antagonistic action, however, as in Patent Literature 1, only an effect exhibited by a neuropathic pain model is shown, and whether the compound has a treatment effect in the irritable bowel syndrome or the inflammatory bowel disease is unclear.

Moreover, the present applicant has also filed patent applications related to the P2X4 receptor antagonist as in Patent Literatures 3 to 9, however, whether the P2X4 receptor antagonist has a treatment effect in the irritable bowel syndrome or the inflammatory bowel disease is unclear in all of the applications.

Furthermore, none of Non Patent Literatures 1 to 6 and Patent Literatures 1 to 12 specifically teaches that the P2X4 receptor antagonist is useful for preventing or treating the nociceptive pain.

### CITATION LIST

### Patent Literature

Patent Literature 1: WO 2008/020651 A
Patent Literature 2: WO 2010/093061 A
Patent Literature 3: WO 2008/023847 A
Patent Literature 4: WO 2012/008478 A
Patent Literature 5: WO 2012/014910 A
Patent Literature 6: WO 2012/017876 A
Patent Literature 7: WO 2013/105608 A
Patent Literature 8: WO 2015/005468 A
Patent Literature 9: WO 2015/005467 A
Patent Literature 10: WO 2019/177117 A
Patent Literature 11: WO 2020/050253 A
Patent Literature 12: WO 2021/049628 A

### Non Patent Literature

Non Patent Literature 1: The Japanese Society of Gastroenterology Evidence-based Clinical Practice Guidelines for Irritable Bowel Syndrome (IBS) 2014
Non Patent Literature 2: The Journal of the Japanese Society of Internal Medicine 98 (1), 5-11, 2009 Jan. 10
Non Patent Literature 3: Katz S (2007) “Mind the Gap”: an unmet need for new therapy in IBD. Journal of clinical gastroenterology 41:799-809.
Non Patent Literature 4: Kawano A, Tsukimoto M, Mori D, Noguchi T, Harada H, Takenouchi T, Kitani H and Kojima S (2012) Regulation of P2X7-dependent inflammatory functions by P2X4 receptor in mouse macrophages. Biochemical and biophysical research communications 420:102-107.
Non Patent Literature 5: Sakaki H, Fujiwaki T, Tsukimoto M, Kawano A, Harada H and Kojima S (2013) P2X4 receptor regulates P2X7 receptor-dependent IL-1beta and IL-18 release in mouse bone marrow-derived dendritic cells. Biochemical and biophysical research communications 432:406-411.
Non Patent Literature 6: Suurvali J, Boudinot P, Kanellopoulos J and Ruutel Boudinot S (2017) P2X4: A fast and sensitive purinergic receptor. Biomedical journal 40:245-256.

### SUMMARY OF INVENTION

### Technical Problem

An object of the present invention is to provide a medicament for preventing or treating nociceptive pain and/or visceral pain.

### Solution to Problem

In order to solve the above problem, the present inventor has conducted intensive research, and as a result, the present inventor found that compounds represented by general formulas (A) to (BII) that have a P2X4 receptor antagonistic action are useful for preventing or treating the nociceptive pain, thus completing the present invention.

That is, the present invention provides a medicament for preventing and/or treating nociceptive pain and/or visceral pain, the medicament including a compound having a P2X4 receptor antagonistic action or a pharmaceutically acceptable salt thereof as an active ingredient.

As the compound having a P2X4 receptor antagonistic action, for example, compounds represented by the following general formulas (A) to (BII) can be used. More preferably, as the compound having a P2X4 receptor antagonistic action, the compound represented by the following general formula (BI) or (BII) can be used.

The medicament provided by the present invention can be used for preventing or treating nociceptive pain, particularly, for preventing or treating inflammatory pain. Furthermore, the medicament provided by the present invention can be used for, for example, preventing or treating the inflammatory pain, in which the inflammatory pain is pain in inflammatory bowel disease. Furthermore, the medicament provided by the present invention can be used for, for example, preventing or treating the inflammatory pain in inflammatory bowel disease, in which the inflammatory pain in inflammatory bowel disease is inflammatory pain in Crohn's disease, ulcerative colitis, simple ulcer, or non-specific multiple ulcers of the small intestine.

Alternatively/Furthermore, the medicament provided by the present invention can be used for, for example, preventing or treating visceral pain, particularly, for preventing or treating visceral pain caused by the intestinal tract. Furthermore, the medicament provided by the present invention can be used for, for example, preventing or treating the visceral pain caused by the intestinal tract, in which the visceral pain caused by the intestinal tract is visceral pain caused by inflammatory bowel disease. Furthermore, the medicament provided by the present invention can be used for, for example, preventing or treating the visceral pain caused by inflammatory bowel disease, in which the visceral pain caused by inflammatory bowel disease is visceral pain caused by Crohn's disease, ulcerative colitis, simple ulcer, or non-specific multiple ulcers of the small intestine.

Furthermore, the medicament provided by the present invention can be used for, for example, preventing or treating the visceral pain, in which the visceral pain is pain after remission or curing of inflammation.

From another viewpoint, the present invention provides use of a compound having a P2X4 receptor antagonistic action or a pharmaceutically acceptable salt thereof for producing the medicament; and a method of preventing or treating nociceptive pain, particularly, a method of preventing or treating inflammatory pain, the method of preventing or treating the inflammatory pain in which the inflammatory pain is pain in inflammatory bowel disease, or the method of preventing or treating the pain in inflammatory bowel disease, in which the inflammatory pain in inflammatory bowel disease is inflammatory pain in Crohn's disease, ulcerative colitis, simple ulcer, or non-specific multiple ulcers of the small intestine, the method including a step of administering the compound having a P2X4 receptor antagonistic action or a pharmaceutically acceptable salt thereof to a mammal including human at an effective dose for the prevention or treatment.

Alternatively/furthermore, the present invention provides use of a compound having a P2X4 receptor antagonistic action or a pharmaceutically acceptable salt thereof for producing the medicament; and a method of preventing or treating visceral pain, particularly, a method of preventing or treating visceral pain caused by the intestinal tract, the method of preventing or treating the visceral pain caused by the intestinal tract in which the visceral pain caused by the intestinal tract is visceral pain caused by inflammatory bowel disease, or the method of preventing or treating the visceral pain caused by inflammatory bowel disease, in which the visceral pain caused by inflammatory bowel disease is visceral pain caused by Crohn's disease, ulcerative colitis, simple ulcer, or non-specific multiple ulcers of small intestine, the method including a step of administering the compound having a P2X4 receptor antagonistic action or a pharmaceutically acceptable salt thereof to a mammal including human at an effective dose for the prevention or treatment.

Furthermore, the present invention provides use of a compound having a P2X4 receptor antagonistic action or a pharmaceutically acceptable salt thereof for producing the medicament; and the method of preventing or treating the visceral pain, in which the visceral pain is pain after remission or curing of inflammation, the method including a step of administering the compound having a P2X4 receptor antagonistic action or a pharmaceutically acceptable salt thereof to a mammal including human at an effective dose for the prevention or treatment.

### Advantageous Effects of Invention

The medicament of the present invention is used as a medicament for preventing or treating nociceptive pain, particularly, is used as a medicament for preventing or treating inflammatory pain, furthermore, is used as, for example, the medicament for preventing or treating the inflammatory pain, in which the inflammatory pain is pain in inflammatory bowel disease, and furthermore, is used as, for example, the medicament for preventing or treating the inflammatory pain in inflammatory bowel disease, in which the inflammatory pain in inflammatory bowel disease is inflammatory pain in Crohn's disease, ulcerative colitis, simple ulcer, or non-specific multiple ulcers of the small intestine; and
alternatively/furthermore, the medicament of the present invention is used as a medicament for preventing or treating visceral pain, particularly, is used as a medicament for preventing or treating visceral pain caused by the intestinal tract, furthermore, is used as, for example, the medicament for preventing or treating the visceral pain caused by the intestinal tract, in which the visceral pain caused by the intestinal tract is visceral pain caused by inflammatory bowel disease, and furthermore, is used as, for example, the medicament for preventing or treating the visceral pain caused by inflammatory bowel disease, in which the visceral pain caused by inflammatory bowel disease is visceral pain caused by Crohn's disease, ulcerative colitis, simple ulcer, or non-specific multiple ulcers of the small intestine, each of which is expected to be highly effective.

The medicament of the present invention is used as the medicament for preventing or treating the visceral pain, in which the visceral pain is pain after remission or curing of inflammation, each of which is expected to be highly effective.

### BRIEF DESCRIPTION OF DRAWINGS

FIG. 1A is a diagram showing an effect of acute administration of Compound A on visceral pain (visceral hypersensitivity) in rats induced by intrarectal injection of 2,4-dinitrobenzenesulfonic acid (DNBS).

FIG. 1B is a diagram showing effects of acute administration of Compound B on visceral pain (visceral hypersensitivity) in rats induced by intrarectal injection of DNBS.

FIG. 2A is a diagram showing an effect of repeated administration (prevention test) of Compound A on visceral pain (visceral hypersensitivity) in rats induced by intrarectal injection of DNBS.

FIG. 2B is a diagram showing an effect of repeated administration (prevention test) of Compound B on visceral pain (visceral hypersensitivity) in rats induced by intrarectal injection of DNBS.

FIG. 2C is a diagram showing an effect of the repeated administration (prevention test) of Compound A on colonic damage in rats induced by the intrarectal injection of DNBS.

FIG. 2D is a diagram showing an effect of the repeated administration (prevention test) of Compound B on colonic damage in rats induced by the intrarectal injection of DNBS.

FIG. 2E is a diagram showing weight loss in rats inducible by the intrarectal injection of DNBS and an effect of the repeated administration (prevention test) of Compound A on the weight loss.

FIG. 2F is a diagram showing weight loss in rats inducible by the intrarectal injection of DNBS and an effect of the repeated administration (prevention test) of Compound B on the weight loss.

FIG. 3A is a diagram showing an effect of repeated administration (intervention test) of Compound A on visceral pain (visceral hypersensitivity) in rats induced by intrarectal injection of DNBS.

FIG. 3B is a diagram showing an effect of repeated administration (intervention test) of Compound B on visceral pain (visceral hypersensitivity) in rats induced the by intrarectal injection of DNBS.

FIG. 3C is a diagram showing an effect of the repeated administration (intervention test) of Compound A on colonic damage in rats induced by the intrarectal injection of DNBS.

FIG. 3D is a diagram showing an effect of the repeated administration (intervention test) of Compound B on colonic damage in rats induced by intrarectal injection of DNBS.

FIG. 3E is a diagram showing weight loss in rats inducible by the intrarectal injection of DNBS and an effect of the repeated administration (intervention test) of Compound A on the weight loss.

FIG. 3F is a diagram showing weight loss in rats inducible by the intrarectal injection of DNBS and an effect of the repeated administration (intervention test) of Compound B on the weight loss.

FIG. 4 is a diagram showing an effect of Compound A on occludin expression in colonic mucosa in rats induced by intrarectal injection of DNBS.

FIG. 5 is a diagram showing an effect of Compound B on occludin expression in colonic mucosa in rats induced by intrarectal injection of DNBS.

### DESCRIPTION OF EMBODIMENTS

In one embodiment, a medicament provided by the present invention can be used as a medicament for preventing or treating nociceptive pain, particularly, inflammatory pain, and can also be used as a medicament for the following use.

In one embodiment, the medicament provided by the present invention can be used as a medicament for preventing or treating inflammatory pain in inflammatory bowel disease.

In one embodiment, the medicament provided by the present invention can be used as a medicament for preventing or treating inflammatory pain in Crohn's disease, ulcerative colitis, simple ulcer, or non-specific multiple ulcers of the small intestine.

In one embodiment, the medicament provided by the present invention can be used as a medicament for preventing or treating visceral pain.

In one embodiment, the medicament provided by the present invention can be used as a medicament for preventing or treating visceral pain caused by the intestinal tract.

In one embodiment, the medicament provided by the present invention can be used as a medicament for preventing or treating visceral pain caused by inflammatory bowel disease.

In one embodiment, the medicament provided by the present invention can be used as a medicament for preventing or treating visceral pain caused by Crohn's disease, ulcerative colitis, simple ulcer, or non-specific multiple ulcers of the small intestine.

In one embodiment, the medicament provided by the present invention can be used as the medicament for preventing or treating the visceral pain, in which the visceral pain is pain after remission or curing of inflammation.

Pain is broadly classified into “somatic pain” (pain on the body surface) and “visceral pain”, according to classification by the site of the pain. Visceral pain is reported as “poorly localized, periodic dull pain or burning sensation”. Regarding stimuli that cause the visceral pain, viscera do not respond to heat stimulation, which is different from somatic pain. Furthermore, rapid expansion, spasmodic contraction, and the like of hollow viscera such as stomach and intestine serve as causes of the visceral pain. Nerves that innervate an impaired organ run together with autonomic nerves, and therefore, are stimulated and frequently accompanied by an autonomic nervous symptom (nausea and vomiting, sweating, tachycardia, or the like).

On the other hand, pain is broadly classified into “nociceptive pain”, which is pain that occurs through a nociceptor, and pain that does not occur through a nociceptor, according to classification by the cause of the pain. The pain that does not occur through a nociceptor is classified into “neuropathic pain” and “psychogenic pain”. Nociceptive pain is generally classified into somatic pain and visceral pain, depending on the site of the pain. A nociceptor refers to a receptor for a stimulus causing pain (that is, a nociceptive stimulus) and can be a receptor for a heat stimulus, a mechanical stimulus, and/or a chemical stimulus.

A nociceptor for somatic pain is present on skin, a subcutaneous tissue, fascia, or other connective tissues, periosteum, endosteum, a joint capsule, or the like, and a nociceptor for the visceral pain is present on most viscera and connective tissues surrounding the viscera. In particular, pain caused by inflammation is referred to as “inflammatory pain” and can be included in the nociceptive pain. “Pain” is known to generally occur with actual tissue damage (for example, inflammation). On the other hand, pain is known to occur even in a case of no actual tissue damage (for example, pain that remains after curing of primary disease). As describe above, rapid expansion, spasmodic contraction, and the like of hollow viscera such as stomach and intestine serve as causes of the visceral pain, however, the visceral pain can be caused even in a case of no actual tissue damage (for example, during remission or after curing of inflammation which is the primary disease) and by extension stimulation or spasm of a smooth muscle, hyperextension of peritoneum, and the like.

In the present specification, the term “prevention” is a concept including preventing onset of a “diseased” or “abnormal” symptom, state, or disease before an outbreak thereof and an action or a method therefor.

In the present specification, particular examples of the “diseased” or “abnormal” symptom, state, or disease can include “pain”, that is, an unpleasant sensory or emotional experience.

In the present specification, the term “treatment” is a concept including eliminating, completely curing, healing, or remitting a “diseased” or “abnormal” symptom, state, or disease and an action or a method therefor, suppressing exacerbation of a “diseased” or “abnormal” symptom, state, or disease and an action or a method therefor, and improvement. Here, the term “improvement” is a concept including approach of a “diseased” or “abnormal” symptom, state, or disease to a “healthy” or “normal” state or an action or a method therefor, and causing a “diseased” or “abnormal” symptom, state, or disease to be in a “healthy” or “normal” state or an action or a method therefor. Therefore, the term “improvement” in one embodiment includes a concept in which a numerical value which serves as an index of a “diseased” or “abnormal” symptom or state becomes small or large so as to approach a normal value or be the normal value in accordance with the “improvement”. Furthermore, the term “suppression” is a concept including stopping or slowing down exacerbation or progression of a symptom, state, or disease and an action or a method therefor, and improving the symptom, state, or disease or an action or a method therefor. Here, the term “improvement” has the meaning described above. The expression “exacerbation or progression of symptom, state, or disease” includes exacerbation or progression of a “diseased” or “abnormal” symptom, state, or disease and exacerbation or progression from a “healthy” or “normal” state to a “diseased” or “abnormal” symptom, state, or disease. The term “suppression” in one embodiment is stopping or slowing down exacerbation or progression of a symptom, state, or disease or an action or a method therefor. The term “suppression” in another embodiment means stopping or slowing down exacerbation or progression of a symptom, state, or disease.

The term “treatment” in one embodiment is eliminating, completely curing, healing, or remitting a “diseased” or “abnormal” symptom, state, or disease and an action or a method therefor. The term “treatment” in another embodiment is eliminating, completely curing, healing, or remitting a “diseased” or “abnormal” symptom, state, or disease.

Therefore, for example, the expression “treatment of pain” is a concept including eliminating, completely curing, healing, or remitting pain and an action or a method therefor, suppressing exacerbation of the pain and an action or a method therefor, and improvement. In one embodiment, the expression “treatment of pain” is eliminating, completely curing, healing, or remitting pain and an action or a method therefor. In another embodiment, the expression “treatment of pain” is eliminating, completely curing, healing, or remitting pain.

In addition, the expression “treatment of pain” can also include a concept of management or control of pain. That is, the treatment of pain aims to “ensure sleep at night without disruption by pain”, “eliminate pain during rest”, or “eliminate pain during body movement” and includes achieving these aims and an action or a method therefor.

In the present specification, the symptom, state, or disease (for example, pain) or a numerical value which serves as an index of these can be evaluated by comparison thereof before and after administration of the medicament provided by the present invention or by comparison thereof between a group to which a placebo or a control that does not contain the medicament provided by the present invention is administered and a group to which the medicament provided by the present invention is administered.

In the present specification, the term “remission induction therapy” means a treatment method conducted for remission of a “diseased” or “abnormal” symptom, state, or disease during onset or exacerbation of the symptom, state, or disease, particularly during an acute phase of the exacerbation. Furthermore, the term “remission maintenance therapy” means a treatment method conducted for maintaining a remitted state of the symptom, state, or disease or for prevention of recurrence of the symptom, state, or disease. “Remission induction therapy” in one embodiment is a treatment method conducted for remission of irritable bowel syndrome or inflammatory bowel disease, or a symptom accompanying the irritable bowel syndrome or the inflammatory bowel disease, and the term “remission maintenance therapy” means a treatment method conducted for maintaining a remitted stated of the irritable bowel syndrome or the inflammatory bowel disease, or a symptom accompanying the irritable bowel syndrome or the inflammatory bowel disease, or for preventing recurrence of the irritable bowel syndrome or the inflammatory bowel disease, or a symptom accompanying the irritable bowel syndrome or the inflammatory bowel disease.

In the present specification, the expression “protection of mucosa” means maintaining or preserving a tissue or function of normal mucosa, or suppressing damage or failure of a tissue or function of mucosa. Here, the term “suppression” has the meaning described above. The expression “maintenance of mucosa” means preserving a tissue or function of normal mucosa, or suppressing deterioration of a tissue or function of mucosa. Here, the term “suppression” has the meaning described above.

In the present specification, the symptom, state, or disease, a numerical value which serves as an index of these, or a state or a function of a tissue of mucosa can be evaluated by comparison thereof before and after administration of the medicament provided by the present invention or by comparison between a group to which a placebo or a control that does not contain the medicament provided by the present invention is not administered and a group to which the medicament provided by the present invention is administered.

In the present specification, the term “suppression” can be used as a term meaning stopping or slowing down increase of supply in a living body.

In the present specification, the expression “maintenance of health of large intestinal tissue” means maintaining or preserving a normal large intestinal tissue or a function thereof, or suppressing damage or failure of a large intestinal tissue or a function thereof. Here, the term “suppression” has the meaning described above. The term “contribution” refers to exhibiting power to cause a certain state or function to become a desired state or function. The expression “contribute to maintenance of health of large intestinal tissue” in one embodiment refers to exhibiting power to cause a large intestinal tissue in a state of being damaged or damaged or a function thereof to be a large intestinal tissue in a normal state or a function thereof.

As an active ingredient of the medicament of the present invention, compounds represented by the following general formulas (A) to (BII) or a pharmaceutically acceptable salt thereof can be used.

Symbols used in the tables below and the like are as follows. Me: methyl group, Et: ethyl group, Pr: n-propyl group, iPr: isopropyl group, tBu: tert-butyl group, Ac: acetyl group, Ph: phenyl group

In the tables below and the like, a substituent may be marked together with a position number indicating a substitution position of the substituent. In addition, in order to distinguish position numbers that appear to be the same in a chemical formula, one position number may be indicated with a prime symbol “′” for convenience, however, as long as a definitive structure for a compound name can be specified, position numbers may be indicated without using the prime symbol.

(A-1) A compound represented by the following general formula (A) or a pharmaceutically acceptable salt thereof:

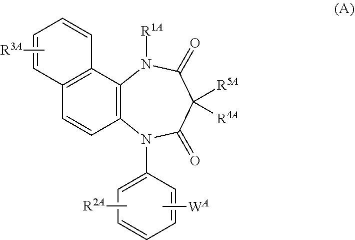

(in the formula, RIA represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, or an alkyl group having 1 to 3 carbon atoms and substituted with a phenyl group;
R^{2A} and R^{3A} may be the same or different, and represent a hydrogen atom, an alkyl group having 1 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, an acylamino group having 2 to 8 carbon atoms and substituted with 1 to 3 halogen atoms, an alkylsulfonylamino group having 1 to 8 carbon atoms, a carboxyl group, an acyl group having 2 to 8 carbon atoms, an alkoxycarbonyl group (the alkoxy moiety has 1 to 8 carbon atoms), a carbamoyl group, an alkylthio group having 1 to 8 carbon atoms, an alkylsulfinyl group having 1 to 8 carbon atoms, an alkylsulfonyl group having 1 to 8 carbon atoms, or a sulfamoyl group;
R^{4A} and R^{5A} may be the same or different, and represent a hydrogen atom, an alkyl group having 1 to 8 carbon atoms, an alkyl group having 1 to 8 carbon atoms and substituted with 1 to 3 halogen atoms, or an alkyl group having 1 to 3 carbon atoms and substituted with a phenyl group; and
W^{A} represents a five or six membered heterocyclic ring including 1 to 4 nitrogen atoms as the members of the ring, which may have a substituent).

In the general formula (A), examples of the alkyl group having 1 to 8 carbon atoms represented by R^{1A}, R^{2A}, R^{3A}, R^{4A}, and R^{5A} can include a methyl group, an ethyl group, a propyl group, an isopropyl group, a butyl group, an i-butyl group, a t-butyl group, a pentyl group, a hexyl group, and the like.

Examples of the alkenyl group having 2 to 8 carbon atoms represented by R^{1A} can include an allyl group and the like.

Examples of the alkyl group having 1 to 8 carbon atoms and substituted with 1 to 3 halogen atoms represented by R^{1A}, R^{2A}, R^{3A}, R^{4A}, and R^{5A} or R^{11A}, R^{12A}, R^{13A}, R^{14A}, and R^{15A} can include a methyl group, an ethyl group, a propyl group, an isopropyl group, a butyl group, a t-butyl group, and the like substituted with 1 to 3 halogen atoms such as a fluorine atom, a chlorine atom, and a bromine atom, and preferable examples thereof can include a trifluoromethyl group, a chloromethyl group, a 2-chloroethyl group, a 2-bromoethyl group, a 2-fluoroethyl group, and the like.

Examples of the alkyl group having 1 to 3 carbon atoms and substituted with a phenyl group represented by R^{1A}, R^{4A}, and R^{5A} can include a benzyl group and the like.

Examples of the alkoxy group having 1 to 8 carbon atoms represented by R^{2A} and R^{3A} can include a methoxy group, an ethoxy group, a propoxy group, an isopropoxy group, a butoxy group, an i-butoxy group, a t-butoxy group, a pentyloxy group, a hexyloxy group, and the like.

Examples of the alkoxy group having 1 to 8 carbon atoms and substituted with 1 to 3 halogen atoms represented by R^{2A} and R^{3A} can include a methyl group, an ethyl group, a propyl group, an isopropyl group, a butyl group, a t-butyl group, and the like substituted with 1 to 3 halogen atoms such as a fluorine atom, a chlorine atom, and a bromine atom, and preferable examples thereof can include a trifluoromethoxy group, a 2-chloroethoxy group, a 2-bromoethoxy group, a 2-fluoroethoxy group, and the like.

Examples of the halogen atom represented by R^{2A} and R^{3A} can include a fluorine atom, a chlorine atom, a bromine atom, and the like.

Examples of the alkylamino group having 1 to 8 carbon atoms represented by R^{2A} and R^{3A} can include a methylamino group, an ethylamino group, and the like.

Examples of the dialkylamino group having 1 to 8 carbon atoms represented by R^{2A} and R^{3A} can include a dimethylamino group, a diethylamino group, and the like.

Examples of the acylamino group having 2 to 8 carbon atoms represented by R^{2A} and R^{3A} can include an acetylamino group.

Examples of the acylamino group having 2 to 8 carbon atoms and substituted with 1 to 3 halogen atoms represented by R^{2A} and R^{3A} can include a trifluoromethylcarbonylamino group.

Examples of the alkylsulfonylamino group having 1 to 8 carbon atoms represented by R^{2A} and R^{3A} can include a methylsulfonylamino group.

Examples of the acyl group having 2 to 8 carbon atoms represented by R^{2A} and R^{3A} can include an acetyl group.

Examples of the alkoxycarbonyl group (the alkoxy moiety has 1 to 8 carbon atoms) represented by R^{2A} and R^{3A} can include a methoxycarbonyl group, an ethoxycarbonyl group, and the like.

Examples of the alkylthio group having 1 to 8 carbon atoms represented by R^{2A} and R^{3A} can include a methylthio group.

Examples of the alkylsulfinyl group having 1 to 8 carbon atoms represented by R^{2A} and R^{3A} can include a methylsulfinyl group.

Examples of the alkylsulfonyl group having 1 to 8 carbon atoms represented by R^{2A} and R^{3A} can include a methylsulfonyl group.

Examples of the five or six membered heterocyclic ring including 1 to 4 nitrogen atoms as the members of the ring, which may have a substituent, represented by W^{A} can include tetrazole, 1,2,4-triazole, 1,2,3-triazole, 1,2,4-oxadiazole, pyrazole, imidazole, oxazole, isoxazole, pyrrole, thiazole, pyridine, and pyrrolidine.

Examples of the substituent that may be included in the five or six membered heterocyclic ring including 1 to 4 nitrogen atoms as the members of the ring, which may have a substituent, represented by W^{A} can include an alkyl group having 1 to 8 carbon atoms such as a methyl group and an ethyl group, an alkyl group having 1 to 8 carbon atoms and substituted with 1 to 3 halogen atoms such as a trifluoromethyl group, a halogen atom such as a fluorine atom, a cyano group, an oxo group, a thioxo group, and the like.

1 to 3 of the same or different R^{2A}s and R^{3A}s in the general formula (A) may be present on a benzene ring substituted with R^{2A} and R^{3A}.

As the compound represented by the general formula (A), the following compounds are preferable.

(A-2) The compound according to (A-1), in which W^{A} represents tetrazole, 1,2,4-triazole, 1,2,3-triazole, 1,2,4-oxadiazole, pyrazole, or imidazole that may have a substituent selected from an alkyl group having 1 to 8 carbon atoms, an alkyl group having 1 to 8 carbon atoms and substituted with 1 to 3 halogen atoms, a halogen atom, a cyano group, an oxo group, and a thioxo group.

(A-3) The compound according to (A-1) or (A-2), in which W^{A} represents tetrazole, 1,2,4-triazole, or 1,2,3-triazole that may have a substituent selected from an alkyl group having 1 to 8 carbon atoms, an alkyl group having 1 to 8 carbon atoms and substituted with 1 to 3 halogen atoms, a halogen atom, and a cyano group.

(A-4) The compound according to any one of (A-1) to (A-3), in which W^{A} represents 5-oxo-1,2,4-oxadiazole or 5-thioxo-1,2,4-oxadiazole.

(A-5) The compound according to any one of (A-1) to (A-4), in which W^{A} represents tetrazole.

(A-6) The compound according to any one of (A-1) to (A-5), in which R^{1A} represents a hydrogen atom or an alkyl group having 1 to 8 carbon atoms.

(A-7) The compound according to any one of (A-1) to (A-6), in which R^{1A} represents a hydrogen atom.

(A-8) The compound according to any one of (A-1) to (A-7), in which R^{4A} represents a hydrogen atom, and R^{5A} represents a hydrogen atom or an alkyl group having 1 to 8 carbon atoms.

(A-9) The compound according to any one of (A-1) to (A-8), in which R^{4A} and R^{5A} both represent hydrogen atoms.

(A-10) The compound according to any one of (A-1) to (A-9), in which R^{2A} represents a hydrogen atom, an alkyl group having 1 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, a carboxyl group, an acyl group having 2 to 8 carbon atoms, or an alkoxycarbonyl group (the alkoxy moiety has 1 to 8 carbon atoms).

(A-11) The compound according to any one of (A-1) to (A-10), in which R^{2A} represents a hydrogen atom.

(A-12) The compound according to any one of (A-1) to (A-11), in which R^{3A} represents a hydrogen atom, an alkyl group having 1 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, a carboxyl group, an acyl group having 2 to 8 carbon atoms, or an alkoxycarbonyl group (the alkoxy moiety has 1 to 8 carbon atoms).

(A-13) The compound according to any one of (A-1) to (A-12), in which R^{3A} represents a hydrogen atom.

Examples of the pharmaceutically acceptable salt of the compound represented by the general formula (A) can include hydrochloride and the like, in a case where R^{2A} and R^{3A} in the general formula (A) represent amino groups or the like. In addition, in a case where R^{2A} and R^{3A} in the general formula (A) represent carboxyl groups, examples of the pharmaceutically acceptable salt of the compound represented by the general formula (A) can include a salt of an alkali metal such as sodium, potassium, and lithium.

Typical compounds included in the compounds represented by the general formula (A) are as follows.

### <Typical Compound A-100>

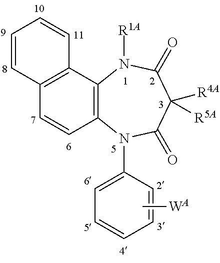

(R^{1A}, R^{4A}, R^{5A}, and W^{A} in the formula and a substitution position of W^{A} are indicated in Tables 1 to 3)

In Tables 1 to 3, the substitution position of W^{A} indicates a substitution position on a benzene ring. That is, position 2, position 3, and position 4 in the tables correspond to position 2′, position 3′, and position 4′ in a formula of Typical Compound A-100, respectively.

TABLE 1

| | Substitution | | |
|---|---|---|---|
| | position | | |
| R^{1A} | of W^{A} | W^{A} | R^{4A}/R^{5A} |
| H | 2- | 1H-Tetrazol-5-yl | H/H |
| H | 3- | 1H-Tetrazol-5-yl | H/H |
| H | 3- | (1-Methyl-1H-terazol)-5-yl | H/H |
| H | 4- | 1H-Tetrazol-5-yl | H/H |
| Me | 3- | 1H-Tetrazol-5-yl | H/H |
| Me | 3- | 1H-Tetrazol-5-yl | Me/H |
| Bn | 3- | 1H-Tetrazol-5-yl | H/H |
| H | 3- | 1H-Tetrazol-1-yl | H/H |
| H | 3- | 1H-Tetrazol-1-yl | Me/Me |
| H | 3- | (1,2,3-Triazol)-5-yl | H/H |
| H | 3- | (1,2,4-Triazol)-3-yl | H/H |
| H | 4- | (1,2,4-Triazol)-3-yl | H/H |

TABLE 2

| | Substitution | | |
|---|---|---|---|
| | position | | |
| R^{1A} | of W^{A} | W^{A} | R^{4A}/R^{5A} |
| H | 2- | (1,2,4-Triazol)-1-yl | H/H |
| H | 3- | (1,2,4-Triazol)-1-yl | H/H |
| H | 3- | [5-(Trifluoromethyl)-1,2,4-triazol]-3-yl | H/H |
| H | 3- | [5-(Trifluoromethyl)-1,2,4-triazol]-3-yl | Et/H |
| H | 3- | [5-Fluoro-1,2,3-triazol]-4-yl | H/H |
| H | 3- | [5-Fluoro-1,2,3-triazol]-4-yl | Me/Me |
| H | 3- | [5-Cyano-1,2,3-triazol]-4-yl | H/H |
| H | 4- | 1H-Imidazol-1-yl | H/H |
| H | 4- | 1H-Imidazol-1-yl | Pr/H |
| H | 3- | 1H-Imidazol-2-yl | H/H |
| H | 3- | 1H-Imidazol-4-yl | H/H |
| H | 3- | Imidazolin-2-yl | H/H |

TABLE 3

| | Substitution | | |
|---|---|---|---|
| | position | | |
| R^{1A} | of W^{A} | W^{A} | R^{4A}/R^{5A} |
| H | 2- | Pyrazol-3-yl | H/H |
| H | 3- | Pyrazol-4-yl | H/H |
| H | 3- | Pyrazol-5-yl | Me/H |
| H | 3- | (1,2,4-Oxadiazol)-3-yl | H/H |
| H | 3- | (1,3,4-Oxadiazol)-2-yl | H/H |
| H | 3- | (5-Oxo-1,2,4-oxadiazol)-3-yl | H/H |
| H | 3- | Pyrrol-1-yl | H/H |
| H | 4- | Pyrrolidin-2-yl | H/H |
| Me | 4- | Pyrrolidin-2-yl | Me/H |
| H | 4- | (1,3-Oxazol)-5-yl | H/H |
| H | 3- | (1,3-Oxazol)-5-yl | H/H |
| H | 2- | (1,3-Thiazol)-5-yl | H/H |

### <Typical Compound A-200>

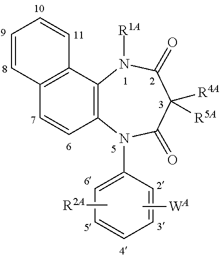

(R^{1A}, R^{2A}, R^{4A}, R^{5A}, and W^{A} in the formula and a substitution position of W^{A} are indicated in Tables 4 and 5)

In Tables 4 and 5, the substitution position of W^{A} indicates a substitution position on a benzene ring. That is, position 2, position 3, and position 4 in the tables correspond to position 2′, position 3′, and position 4′ in a formula of Typical Compound A-200, respectively.

TABLE 4

| | | Substitution | | |
|---|---|---|---|---|
| | | position | | |
| R^{1A} | R^{2A} | of W^{A} | W^{A} | R^{4A}/R^{5A} |
| H | 4-OH | 3- | 1H-Tetrazol-5-yl | H/H |
| H | 4-OMe | 3- | 1H-Tetrazol-5-yl | H/H |
| Me | 2-Cl | 3- | 1H-Tetrazol-5-yl | H/H |
| H | 2,6-Cl | 3- | 1H-Tetrazol-5-yl | H/H |
| H | 4-F | 3- | 1H-Tetrazol-5-yl | H/H |
| H | 4-Br | 3- | 1H-Tetrazol-5-yl | Et/H |
| H | 3-OMe | 4- | (1-Methyl-1H-terazol)-5-yl | H/H |
| H | 4-Me | 3- | 1H-Tetrazol-5-yl | H/H |

TABLE 5

| | | Substitution | | |
|---|---|---|---|---|
| | | position | | |
| R^{1A} | R^{2A} | of W^{A} | W^{A} | R^{4A}/R^{5A} |
| H | 4-Cl | 3- | (1,2,3-Triazol)-5-yl | Me/H |
| H | 4-CF₃ | 3- | (1,2,3-Triazol)-5-yl | H/H |
| H | 3-SMe | 4- | (1,2,4-Triazol)-1-yl | H/H |
| H | 3-SO₂Me | 4- | 1H-Imidazol-1-yl | H/H |
| H | 3-NHSO₂Me | 4- | 1H-Imidazol-1-yl | H/H |
| H | 4-OMe | 3- | 1H-Imidazol-4-yl | H/H |
| H | 4-F | 2- | Pyrazol-3-yl | H/H |

### <Typical Compound A-300>

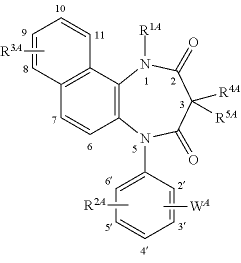

(R^{1A}, R^{2A}, R^{3A}, R^{4A}, R^{5A} and W^{A} in the formula and a substitution position of W^{A} are indicated in Tables 6 and 7)

In Tables 6 and 7, the substitution position of W^{A} indicates a substitution position on a benzene ring. That is, position 3 and position 4 in the tables correspond to position 3′ and position 4′ in a formula of Typical Compound A-300, respectively.

TABLE 6

| | | Substitution | | | |
|---|---|---|---|---|---|
| | | position | | | |
| R^{1A} | R^{2A} | of W^{A} | W^{A} | R^{3A} | R^{4A}/R^{5A} |
| H | H | 3- | 1H-Tetrazol-5-yl | 9-Br | H/H |
| H | 4-OMe | 3- | 1H-Tetrazol-5-yl | 9-Cl | H/H |
| H | 4-OH | 3- | 1H-Tetrazol-5-yl | 10-OMe | H/H |
| H | 2-Cl | 3- | 1H-Tetrazol-5-yl | 9-Br | H/H |
| H | 2,6-Cl | 3- | 1H-Tetrazol-5-yl | 9-Me | H/H |
| H | H | 3- | 1H-Tetrazol-5-yl | 10-Cl | Me/H |
| H | 3-OMe | 4- | (1-Methyl-1H- | 9-CF₃ | H/H |
| | | | terazol)-5-yl | | |

TABLE 7

| | | Substitution | | | |
|---|---|---|---|---|---|
| | | position | | | |
| R^{1A} | R^{2A} | of W^{A} | W^{A} | R^{3A} | R^{4A}/R^{5A} |
| H | 4-Me | 3- | 1H-Tetrazol-1-yl | 9-CN | Pr/H |
| Me | H | 3- | (1,2,3-Triazol)-5-yl | 9-OH | H/H |
| Et | H | 3- | (1,2,3-Triazol)-5-yl | 10-F | H/H |
| H | 3-Br | 4- | (1,2,4-Triazol)-1-yl | 9-SMe | H/H |
| Allyl | H | 4- | 1H-Imidazol-1-yl | 8-OMe | H/H |
| H | H | 3- | 1H-Imidazol-1-yl | 10-OMe | Me/Me |

(B-1) A compound represented by the following general formula (BI) or a pharmaceutically acceptable salt thereof:

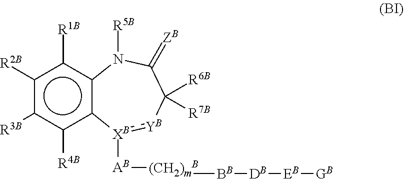

(in the formula, R^{1B} and R^{2B} may be the same or different, and represent a hydrogen atom, an alkyl group having 1 to 8 carbon atoms, a cycloalkyl group having 3 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), a phenyl group which may be substituted, a pyridyl group which may be substituted, or an aralkyl group (the aryl moiety has 6 to 10 carbon atoms, and the alkylene moiety has 1 to 8 carbon atoms), or
R^{1B} and R^{2B} may bind together to form a condensed ring selected from a naphthalene ring, a quinoline ring, an isoquinoline ring, a tetrahydronaphthalene ring, an indane ring, a tetrahydroquinoline ring, and a tetrahydroisoquinoline ring together with the benzene ring to which they bind, and the ring constituted by R^{1B} and R^{2B} bound to each other, together with the carbon atoms to which R^{1B} and R^{2B} bind may be substituted with 1 to 4 of the same or different substituents selected from an alkyl group having 1 to 8 carbon atoms, a cycloalkyl group having 3 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), and an aralkyl group (the aryl moiety has 6 to 10 carbon atoms, and the alkylene moiety has 1 to 8 carbon atoms),
R^{3B} and R^{4B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), or an aralkyl group (the aryl moiety has 6 to 10 carbon atoms, and the alkylene moiety has 1 to 8 carbon atoms),
R^{5B} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
R^{6B} and R^{7B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, or an amino group,
X^{B} represents C, CH, or N,
Y^{B} represents N, NH, or C(═O),
provided that when X^{B} is N, Y^{B} is not N or NH, and
when X^{B} is C or CH, Y^{B} is not C(═O),
the double line consisting of the solid line and the broken line represents a single bond or a double bond,
Z^{B} represents an oxygen atom or a sulfur atom,
A^{B} represents a benzene ring, a pyridine ring, a thiophene ring, a pyrimidine ring, a naphthalene ring, a quinoline ring, or an indole ring, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an aralkyl group (the aryl moiety has 6 to 10 carbon atoms, and the alkylene moiety has 1 to 8 carbon atoms), a phenyl group, and a pyridyl group, as a substituent, or represents an atomic bond,
B^{B} represents N(R^{8B})C(═O), NHCONH, CON(R^{9B}), NHC(═S)NH, N(R^{10B})SO₂, SO₂N(R^{11B}), or OSO₂, wherein
R^{8B}, R^{9B}, R^{10B}, and R^{11B} represent a hydrogen atom, an alkyl group having 1 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
D^{B} represents an alkylene chain having 1 to 6 carbon atoms, which may have 1 to 4 of the same or different substituents selected from an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with a hydroxyl group, and an aralkyl group (the aryl moiety has 6 to 10 carbon atoms, and the alkylene moiety has 1 to 8 carbon atoms), as a substituent, and may further have a double bond, or represents an atomic bond,
E^{B} represents O, S, NR^{12B}, or an atomic bond, wherein
R^{12B} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
G^{B} represents piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, an alkylsulfonyl group having 1 to 6 carbon atoms, an aralkyl group (the aryl moiety has 6 to 10 carbon atoms, and the alkylene moiety has 1 to 8 carbon atoms), a phenyl group which may be substituted, a pyridyl group which may be substituted, an imidazolyl group which may be substituted, an oxazolyl group which may be substituted, and a thiazolyl group which may be substituted, as a substituent, and
m^{B} represents an integer of 0 to 5,
provided that when R^{1B} and R^{2B} do not bind together to form a ring, those compounds are excluded wherein, X^{B} is C, Y^{B} is N, the double line consisting of the solid line and the broken line is a double bond, Z^{B} is an oxygen atom, A^{B} is a benzene ring, m^{B} is 0, B^{B} is C(═O)NH, E^{B} is an atomic bond, and G^{B} is a phenyl group).

(B-2) A compound represented by the following general formula (BII) or a pharmaceutically acceptable salt thereof:

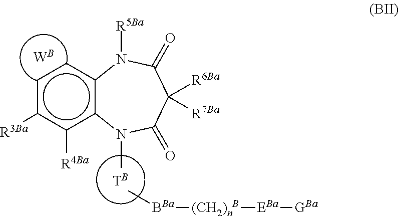

(In the formula,

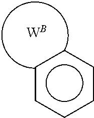

represents a naphthalene ring, a quinoline ring, an isoquinoline ring, a tetrahydronaphthalene ring, an indane ring, a tetrahydroquinoline ring, or a tetrahydroisoquinoline ring, and
these rings may be substituted with 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), and an aralkyl group (the aryl moiety has 6 to 10 carbon atoms, and the alkylene moiety has 1 to 8 carbon atoms),
R^{3Ba} and R^{4Ba} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), or an aralkyl group (the aryl moiety has 6 to 10 carbon atoms, and the alkylene moiety has 1 to 8 carbon atoms),
R^{5Ba} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
R^{6Ba} and R^{7Ba} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, or an amino group,

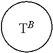

represents a benzene ring, a pyridine ring, a thiophene ring, a pyrimidine ring, a naphthalene ring, a quinoline ring, or an indole ring, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an aralkyl group (the aryl moiety has 6 to 10 carbon atoms, and the alkylene moiety has 1 to 8 carbon atoms), a phenyl group, and a pyridyl group, as a substituent,
B^{Ba} represents N(R^{8Ba})C(═O), NHCONH, CON(R^{9Ba}), NHC(═S)NH, N(R^{10Ba}) SO₂, SO₂N(R^{11Ba}), or OSO₂, wherein
R^{8Ba}, R^{9Ba}, R^{10Ba}, and R^{11Ba} represent a hydrogen atom, an alkyl group having 1 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
E^{Ba} represents O, S, NR^{12Ba}, or an atomic bond, wherein
R^{12Ba} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with a hydroxyl group, or an aralkyl group (the aryl moiety has 6 to 10 carbon atoms, and the alkylene moiety has 1 to 8 carbon atoms),
G^{Ba} represents piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, an alkylsulfonyl group having 1 to 6 carbon atoms, an aralkyl group (the aryl moiety has 6 to 10 carbon atoms, and the alkylene moiety has 1 to 8 carbon atoms), a phenyl group which may be substituted, a pyridyl group which may be substituted, an imidazolyl group which may be substituted, an oxazolyl group which may be substituted, and a thiazolyl group which may be substituted, as a substituent, and
n^{B} represents an integer of 0 to 5).

Next, the substituents in the general formulas (BI) and (BII) of the present specification is described.

Examples of the alkyl group having 1 to 8 carbon atoms can include a methyl group, an ethyl group, a propyl group, an isopropyl group, a butyl group, an i-butyl group, a t-butyl group, a pentyl group, a hexyl group, and the like.

Examples of the cycloalkyl group having carbon atoms 3 to 8 can include a cyclopropyl group, a cyclohexyl group, and the like.

Examples of the alkenyl group having 2 to 8 carbon atoms can include an allyl group, and the like.

Examples of the alkoxy group having 1 to 8 carbon atoms can include a methoxy group, an ethoxy group, a propoxy group, an isopropoxy group, a butoxy group, an i-butoxy group, a t-butoxy group, a pentyloxy group, a hexyloxy group, and the like.

Examples of the alkyl group having 1 to 8 carbon atoms and substituted with 1 to 3 halogen atoms can include a methyl group, an ethyl group, a propyl group, an isopropyl group, a butyl group, a t-butyl group, and the like substituted with 1 to 3 halogen atoms such as a fluorine atom, a chlorine atom, and a bromine atom, and preferable examples thereof can include a trifluoromethyl group, a chloromethyl group, a 2-chloroethyl group, a 2-bromoethyl group, a 2-fluoroethyl group, and the like.

Examples of the alkoxy group having 1 to 8 carbon atoms substituted with 1 to 3 halogen atoms can include a methoxy group, an ethoxy group, a propoxy group, an isopropoxy group, a butoxy group, a t-butoxy group, and the like substituted with 1 to 3 halogen atoms such as a fluorine atom, a chlorine atom, and a bromine atom, and preferable examples thereof can include a trifluoromethoxy group, a chloromethoxy group, a 2-chloroethoxy group, a 2-bromoethoxy group, a 2-fluoroethoxy group, and the like.

Examples of the halogen atom can include a fluorine atom, a chlorine atom, a bromine atom, and the like.

Examples of the alkylamino group having 1 to 8 carbon atoms can include a methylamino group, an ethylamino group, and the like.

Examples of the dialkylamino group having 2 to 8 carbon atoms can include a dimethylamino group, a diethylamino group, and the like.

Examples of the acylamino group having 2 to 8 carbon atoms can include an acetylamino group and the like.

Examples of the acyl group having 2 to 8 carbon atoms can include an acetyl group and the like.

Examples of the alkoxycarbonyl group (the alkoxy moiety has 1 to 8 carbon atoms) can include a methoxycarbonyl group and the like.

Examples of the aralkyl group (the aryl moiety has 6 to 10 carbon atoms, and the alkylene moiety has 1 to 8 carbon atoms) can include a benzyl group and the like.

Examples of the alkyl group having 1 to 8 carbon atoms and substituted with a hydroxyl group can include a 2-hydroxyethyl group and the like.

Examples of the alkylsulfinyl group having 1 to 6 carbon atoms can include a methanesulfinyl group and the like.

Examples of the alkylthio group having 1 to 6 carbon atoms can include a methylthio group and the like.

Examples of the alkylsulfonyl group having 1 to 6 carbon atoms can include a methanesulfonyl group and the like.

Examples of the substituent that may be included in the phenyl group which may be substituted, the pyridyl group which may be substituted, the imidazolyl group which may be substituted, the oxazolyl group which may be substituted, and the thiazolyl group which may be substituted can include a halogen atom, an alkyl group having 1 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, and the like.

As the compound represented by the general formula (BI), the following compounds are preferable.

### (B-1-1)

The compound according to (B-1), in which R^{1B} and R^{2B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, a phenyl group which may be substituted, a pyridyl group which may be substituted, or an aralkyl group (the aryl moiety has 6 to 10 carbon atoms, and the alkylene moiety has 1 to 8 carbon atoms).

### (B-1-2)

The compound according to (B-1) or (B-1-1), in which R^{1B} and R^{2B} bind together to form a naphthalene ring or a tetrahydronaphthalene ring together with the benzene ring to which they bind, and the benzene ring or the cyclohexene ring constituted by R^{1B} and R^{2B}, bound to each other, together with the carbon atoms to which R^{1B} and R^{2B} bind may be substituted with 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), and an aralkyl group (the aryl moiety has 6 to 10 carbon atoms, and the alkylene moiety has 1 to 8 carbon atoms).

### (B-1-3)

The compound according to (B-1) or (B-1-1), in which R^{1B} and R^{2B} bind together to form a naphthalene ring together with the benzene ring to which they bind, and the benzene ring constituted by R^{1B} and R^{2B}, bound to each other, together with the carbon atoms to which R^{1B} and R^{2B} bind may be substituted with 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, and an amino group.

### (B-1-4)

The compound according to (B-1) or (B-1-1), in which R^{1B} and R^{2B} bind together to form a naphthalene ring or a tetrahydronaphthalene ring together with the benzene ring to which they bind.

### (B-1-5)

The compound according to any one of (B-1) and (B-1-1) to (B-1-4), in which R^{3B} and R^{4B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, or an aralkyl group (the aryl moiety has 6 to 10 carbon atoms, and the alkylene moiety has 1 to 8 carbon atoms).

### (B-1-6)

The compound according to any one of (B-1) and (B-1-1) to (B-1-5), in which R^{5B} represents a hydrogen atom, an alkyl group having 1 to 8 carbon atoms, or an aralkyl group (the aryl moiety has 6 to 10 carbon atoms, and the alkylene moiety has 1 to 8 carbon atoms).

### (B-1-7)

The compound according to any one of (B-1) and (B-1-1) to (B-1-6), in which R^{5B} represents a hydrogen atom.

### (B-1-8)

The compound according to any one of (B-1) and (B-1-1) to (B-1-7), in which R^{6B} and R^{7B} may be the same or different, and represent a hydrogen atom, an alkyl group having 1 to 8 carbon atoms, an alkoxy group having 1 to 8 carbon atoms, an alkyl group having 1 to 8 carbon atoms and substituted with 1 to 3 halogen atoms, or an alkoxy group having 1 to 8 carbon atoms and substituted with 1 to 3 halogen atoms, or a pharmaceutically acceptable salt thereof.

### (B-1-9)

The compound according to any one of (B-1) and (B-1-1) to (B-1-8), in which R^{6B} and R^{7B} both represent hydrogen atoms.

### (B-1-10)

The compound according to any one of (B-1) and (B-1-1) to (B-1-9), in which R^{3B}, R^{4B}, R^{5B}, R^{6B}, and R^{7B} are hydrogen atoms.

### (B-1-11)

The compound according to any one of (B-1) and (B-1-1) to (B-1-10), in which X^{B} represents N, Y^{B} represents C(═O), and the double line consisting of the solid line and the broken line represents a single bond.

### (B-1-12)

The compound according to any one of (B-1) and (B-1-1) to (B-1-11), in which X^{B} represents C, Y^{B} represents N, and the double line consisting of the solid line and the broken line represents a double bond.

### (B-1-13)

The compound according to any one of (B-1) and (B-1-1) to (B-1-12), in which Z^{B} represents an oxygen atom.

### (B-1-14)

The compound according to any one of (B-1) and (B-1-1) to (B-1-13), in which A^{B} represents a phenyl group or a pyridyl group that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an aralkyl group (the aryl moiety has 6 to 10 carbon atoms, and the alkylene moiety has 1 to 8 carbon atoms), a phenyl group, and a pyridyl group as a substituent.

### (B-1-15)

The compound according to any one of (B-1) and (B-1-1) to (B-1-14), in which A^{B} represents a phenyl group that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, and an amino group as a substituent.

### (B-1-16)

The compound according to any one of (B-1) and (B-1-1) to (B-1-15), in which A^{B} represents a phenyl group or a pyridyl group.

### (B-1-17)

The compound according to any one of (B-1) and (B-1-1) to (B-1-16), in which A^{B} represents an atomic bond.

### (B-1-18)

The compound according to any one of (B-1) and (B-1-1) to (B-1-17), in which B^{B} represents NHC(═O), NHCONH, CONH, NHC(═S)NH, NHSO₂, SO₂NH, or OSO₂.

### (B-1-19)

The compound according to any one of (B-1) and (B-1-1) to (B-1-18), in which B^{B} represents NHC(═O), NHCONH, or NHSO₂.

### (B-1-20)

The compound according to any one of (B-1) and (B-1-1) to (B-1-19), in which B^{B} represents NHC(═O) or NHSO₂.

### (B-1-21)

The compound according to any one of (B-1) and (B-1-1) to (B-1-20), in which B^{B} represents NHC(═O).

### (B-1-22)

The compound according to any one of (B-1) and (B-1-1) to (B-1-21), in which D^{B} represents an alkylene chain having 1 to 6 carbon atoms that may have 1 to 4 of the same or different substituents selected from an alkyl group having 1 to 8 carbon atoms and an alkyl group having 1 to 8 carbon atoms and substituted with 1 to 3 halogen atoms as a substituent, and may further have a double bond.

### (B-1-23)

The compound according to any one of (B-1) and (B-1-1) to (B-1-22), in which D^{B} represents an atomic bond.

### (B-1-24)

The compound according to any one of (B-1) and (B-1-1) to (B-1-23), in which D^{B} has 1 to 4 of the same or different substituents selected from an alkyl group having 1 to 8 carbon atoms and an alkenyl group having 2 to 8 carbon atoms as a substituent.

### (B-1-25)

The compound according to any one of (B-1) and (B-1-1) to (B-1-24), in which D^{B} has 1 to 4 of the same or different substituents selected from an alkyl group having 1 to 3 carbon atoms and an alkenyl group having 2 or 3 carbon atoms as a substituent.

### (B-1-26)

The compound according to any one of (B-1) and (B-1-1) to (B-1-25), in which E^{B} represents an atomic bond.

### (B-1-27)

The compound according to any one of (B-1) and (B-1-1) to (B-1-26), in which G^{B} represents piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, and an alkylsulfonyl group having 1 to 6 carbon atoms as a substituent.

### (B-1-28)

The compound according to any one of (B-1) and (B-1-1) to (B-1-27), in which G^{B} represents benzene that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, and an alkylsulfonyl group having 1 to 6 carbon atoms as a substituent.

### (B-1-29)

The compound according to any one of (B-1) and (B-1-1) to (B-1-28), in which G^{B} represents benzene or pyridine that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, a halogen atom, a hydroxyl group, a nitro group, an amino group, a dialkylamino group having 2 to 8 carbon atoms, a carboxyl group, an alkylsulfinyl group having 1 to 6 carbon atoms, an alkylthio group having 1 to 6 carbon atoms, and an alkylsulfonyl group having 1 to 6 carbon atoms as a substituent.

### (B-1-30)

The compound according to any one of (B-1) and (B-1-1) to (B-1-29), in which G^{B} represents benzene that may have 1 to 4 of the same or different substituents selected from an alkyl group having 1 to 8 carbon atoms and substituted with 1 to 3 halogen atoms, a halogen atom, and a hydroxyl group as a substituent.

### (B-1-31)

The compound according to any one of (B-1) and (B-1-1) to (B-1-30), in which m^{B} represents 0.

### (B-1-32)

The compound according to any one of (B-1) and (B-1-1) to (B-1-31), in which A^{B} represents a benzene ring, m^{B} represents 0, B^{B} represents NHC(═O) or NHSO₂, D^{B} represents an alkyl group having 1 to 3 carbon atoms or an atomic bond, E^{B} represents an atomic bond, and G^{B} represents benzene that may have 1 to 4 of the same or different substituents selected from an alkyl group having 1 to 8 carbon atoms, an alkyl group having 1 to 8 carbon atoms and substituted with 1 to 3 halogen atoms, a halogen atom, and a hydroxyl group as a substituent.

### (B-1-33)

The compound according to any one of (B-1) and (B-1-1) to (B-1-32), in which A^{B} represents a benzene ring, m^{B} represents 0, B^{B} represents NHC(═O), D^{B} represents an atomic bond, E^{B} represents an atomic bond, and G^{B} represents benzene that may have 1 to 4 of the same or different substituents selected from an alkyl group having 1 to 8 carbon atoms and substituted with 1 to 3 halogen atoms, a halogen atom, and a hydroxyl group as a substituent.

### (B-1-34)

The compound according to any one of (B-1) and (B-1-1) to (B-1-33), in which R^{1B} and R^{2B} bind together to form a naphthalene ring together with the benzene ring to which they bind, R^{3B}, R^{4B}, R^{5B}, R^{6B}, and R^{7B} represent hydrogen atoms, X^{B} represents N, Y^{B} represents C(═O), the double line consisting of the solid line and the broken line represents a single bond, Z^{B} represents an oxygen atom, A^{B} represents a benzene ring, m^{B} represents 0, B^{B} represents NHC(═O) or NHSO₂, D^{B} represents an alkyl group having 1 to 3 carbon atoms or an atomic bond, E^{B} represents an atomic bond, and G^{B} represents benzene that may have 1 to 4 of the same or different substituents selected from an alkyl group having 1 to 8 carbon atoms, an alkyl group having 1 to 8 carbon atoms and substituted with 1 to 3 halogen atoms, a halogen atom, and a hydroxyl group as a substituent.

### (B-1-35)

The compound according to any one of (B-1) and (B-1-1) to (B-1-34), in which, in the general formula (BI), R^{1B} and R^{2B} bind together to form a naphthalene ring together with the benzene ring to which they bind, R^{3B}, R^{4B}, R^{3B}, R^{6B}, and R^{7B} represent hydrogen atoms, X^{B} represents N, Y^{B} represents C(═O), the double line consisting of the solid line and the broken line represents a single bond, Z^{B} represents an oxygen atom, A^{B} represents a benzene ring, m^{B} represents 0, B^{B} represents NHC(═O), D^{B} represents an atomic bond, E^{B} represents an atomic bond, and G^{B} represents benzene that may have 1 to 4 of the same or different substituents selected from an alkyl group having 1 to 8 carbon atoms and substituted with 1 to 3 halogen atoms, a halogen atom, and a hydroxyl group as a substituent.

As the compound represented the general formula (BII), the following compounds are preferable.

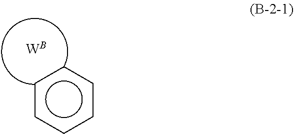

The compound according to (B-2) in which the above moiety represents a naphthalene ring or a tetrahydronaphthalene ring that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), and an aralkyl group (the aryl moiety has 6 to 10 carbon atoms, and the alkylene moiety has 1 to 8 carbon atoms) as a substituent.

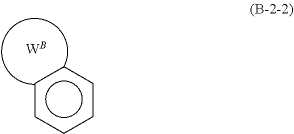

The compound according to (B-2) or (B-2-1) in which the above moiety represents a naphthalene ring that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, and an amino group as a substituent.

### (B-2-3)

The compound according to any one of (B-2), (B-2-1), and (B-2-2) in which R^{3Ba} and R^{4Ba} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, or an aralkyl group (the aryl moiety has 6 to 10 carbon atoms, and the alkylene moiety has 1 to 8 carbon atoms).

### (B-2-4)

The compound according to any one of (B-2) and (B-2-1) to (B-2-3), in which R^{5Ba} represents a hydrogen atom, an alkyl group having 1 to 8 carbon atoms, or an aralkyl group (the aryl moiety has 6 to 10 carbon atoms, and the alkylene moiety has 1 to 8 carbon atoms).

### (B-2-5)

The compound according to any one of (B-2) and (B-2-1) to (B-2-4), in which R^{5Ba} represents a hydrogen atom.

### (B-2-6)

The compound according to any one of (B-2) and (B-2-1) to (B-2-5), in which R^{6Ba} and R^{7Ba} may be the same or different, and represent a hydrogen atom, an alkyl group having 1 to 8 carbon atoms, an alkoxy group having 1 to 8 carbon atoms, an alkyl group having 1 to 8 carbon atoms and substituted with 1 to 3 halogen atoms, or an alkoxy group having 1 to 8 carbon atoms and substituted with 1 to 3 halogen atoms.

### (B-2-7)

The compound according to any one of (B-2) and (B-2-1) to (B-2-6), in which R^{6Ba} and R^{7Ba} both represent hydrogen atoms.

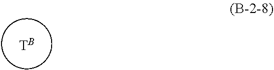

The compound according to any one of (B-2) and (B-2-1) to (B-2-7), in which the above moiety represents a phenyl group or a pyridyl group that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an aralkyl group (the aryl moiety has 6 to 10 carbon atoms, and the alkylene moiety has 1 to 8 carbon atoms), a phenyl group, and a pyridyl group as a substituent.

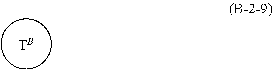

The compound according to any one of (B-2) and (B-2-1) to (B-2-8), in which the above moiety represents a phenyl group that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, and an amino group as a substituent.

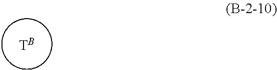

The compound according to any one of (B-2) and (B-2-1) to (B-2-9), in which the above moiety represents an atomic bond.

### (B-2-11)

The compound according to any one of (B-2) and (B-2-1) to (B-2-10), in which B^{Ba} represents NHC(═O), NHCONH, CONH, NHC(═S)NH, NHSO₂, SO₂NH, or OSO₂.

### (B-2-12)

The compound according to any one of (B-2) and (B-2-1) to (B-2-11), in which B^{Ba} represents NHC(═O), NHCONH, or NHSO₂.

### (B-2-13)

The compound according to any one of (B-2) and (B-2-1) to (B-2-12), in which E^{Ba} represents an atomic bond.

### (B-2-14)

The compound according to any one of (B-2) and (B-2-1) to (B-2-13), in which G^{Ba} represents piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, and an alkylsulfonyl group having 1 to 6 carbon atoms as a substituent.

### (B-2-15)

The compound according to any one of (B-2) and (B-2-1) to (B-2-14), in which G^{Ba} represents benzene that may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, and an alkylsulfonyl group having 1 to 6 carbon atoms as a substituent.

### (B-2-16)

The compound according to any one of (B-2) and (B-2-1) to (B-2-15), in which n^{B} represents 0.

In the general formula (BI), it is preferable that R^{B1} and R^{B2} bind together to form a condensed ring selected from a naphthalene ring or a tetrahydronaphthalene ring together with the benzene ring to which they bind, and it is particularly preferable that R^{B1} and R^{B2} bind together to form a naphthalene ring together with the benzene ring to which they bind.

In the general formula (BI), it is preferable that R^{B3}, R^{B4}, R^{B5}, R^{B6}, and R^{B7} represent hydrogen atoms.

In the general formula (BI), it is preferable that X^{B} represents N, Y^{B} represents C(═O), and the double line consisting of the solid line and the broken line represents a single bond.

In the general formula (BI), it is preferable that Z^{B} represents an oxygen atom.

In the general formula (BI), it is preferable that A^{B} represents a benzene ring or a pyridine ring, and it is particularly preferable that A^{B} represents a benzene ring.

In the general formula (BI), it is preferable that m^{B} represents 0 to 4, and it is particularly preferable that m^{B} represents 0.

In the general formula (BI), it is preferable that B^{B} represents N(R^{8B})C(═O) or N(R^{10B})SO₂, and in this case, it is more preferable that R^{8B} and R^{10B} represent hydrogen atoms. Furthermore, in the general formula (BI), it is particularly preferable that B^{B} represents NHC(═O).

In the general formula (BI), it is preferable that D^{B} represents an alkylene chain which has 1 to 4 of the same or different substituents selected from an alkyl group having 1 to 8 carbon atoms and an alkenyl group having 2 to 8 carbon atoms as a substituent, or represents an atomic bond, it is more preferable that D^{B} represents an alkylene chain which has 1 to 4 of the same or different substituents selected from an alkyl group having 1 to 3 carbon atoms and an alkenyl group having 2 or 3 carbon atoms as a substituent, or represents an atomic bond, and it is particularly preferable that D^{B} represents an atomic bond.

In the general formula (BI), it is preferable that E^{B} represents O or an atomic bond, and it is particularly preferable that E^{B} represents an atomic bond.

In the general formula (BI), it is preferable that G^{B} represents benzene or pyridine which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, a halogen atom, a hydroxyl group, a nitro group, an amino group, a dialkylamino group having 2 to 8 carbon atoms, a carboxyl group, an alkylsulfinyl group having 1 to 6 carbon atoms, an alkylthio group having 1 to 6 carbon atoms, and an alkylsulfonyl group having 1 to 6 carbon atoms as a substituent, and it is particularly preferable that G^{B} represents benzene which may have 1 to 4 of the same or different substituents selected from an alkyl group having 1 to 8 carbon atoms and substituted with 1 to 3 halogen atoms, a halogen atom, and a hydroxyl group as a substituent.

In the general formula (BI), it is particularly preferable that A^{B} represents a benzene ring, m^{B} represents 0, B^{B} represents NHC(═O) or NHSO₂, D^{B} represents an alkyl group having 1 to 3 carbon atoms or an atomic bond, E^{B} represents an atomic bond, and G^{B} represents benzene which may have 1 to 4 of the same or different substituents selected from an alkyl group having 1 to 8 carbon atoms, an alkyl group having 1 to 8 carbon atoms and substituted with 1 to 3 halogen atoms, a halogen atom, and a hydroxyl group as a substituent.

In the general formula (BI), it is particularly preferable that A^{B} represents a benzene ring, m^{B} represents 0, B^{B} represents NHC(═O), D^{B} represents an atomic bond, E^{B} represents an atomic bond, and G^{B} represents benzene which may have 1 to 4 of the same or different substituents selected from an alkyl group having 1 to 8 carbon atoms and substituted with 1 to 3 halogen atoms, a halogen atom, and a hydroxyl group as a substituent.

In the general formula (BI), it is more preferable that R^{B1} and R^{B2} bind together to form a naphthalene ring together with the benzene ring to which they bind, R^{B3}, R^{B4}, R^{B5}, R^{B6}, and R^{B7} represent hydrogen atoms, X^{B} represents N, Y^{B} represents C(═O), the double line consisting of the solid line and the broken line represents a single bond, Z^{B} represents an oxygen atom, A^{B} represents a benzene ring, m^{B} represents 0, B^{B} represents NHC(═O) or NHSO₂, D^{B} represents an alkyl group having 1 to 3 carbon atoms or an atomic bond, E^{B} represents an atomic bond, and G^{B} represents benzene which may have 1 to 4 of the same or different substituents selected from an alkyl group having 1 to 8 carbon atoms, an alkyl group having 1 to 8 carbon atoms and substituted with 1 to 3 halogen atoms, a halogen atom, and a hydroxyl group as a substituent.

In the general formula (BI), it is particularly preferable that R^{B1} and R^{B2} bind together to form a naphthalene ring together with the benzene ring to which they bind, R^{B3}, R^{B4}, R^{B5}, R^{B6}, and R^{B7} represent hydrogen atoms, X^{B} represents N, Y^{B} represents C(═O), the double line consisting of the solid line and the broken line represents a single bond, Z^{B} represents an oxygen atom, A^{B} represents a benzene ring, m^{B} represents 0, B^{B} represents NHC(═O), D^{B} represents an atomic bond, E^{B} represents an atomic bond, and G^{B} represents benzene which may have 1 to 4 of the same or different substituents selected from an alkyl group having 1 to 8 carbon atoms and substituted with 1 to 3 halogen atoms, a halogen atom, and a hydroxyl group as a substituent.

Typical compounds included in the compounds represented by the general formulas (BI) and/or (BII) are as follows.

### <Typical Compound Example B-100>

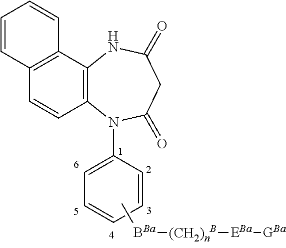

(B^{Ba} (substitution position), n^{B}, E^{Ba}, and G^{Ba} in the formula are indicated in Tables 8 to 17)

TABLE 8

| B^{Ba} (substitution | | | |
|---|---|---|---|
| position) | n^{B} | E^{Ba} | G^{Ba} |
| NHCO(4) | 0 | Atomic bond | Phenyl |
| NHCO(4) | 0 | Atomic bond | (2-CF₃)Phenyl |
| NHCO(4) | 0 | Atomic bond | (3-Br)Phenyl |
| NHCO(4) | 0 | Atomic bond | (4-CF₃)Phenyl |
| NHCO(4) | 0 | Atomic bond | (2-Me)Phenyl |
| NHCO(4) | 0 | Atomic bond | (2,6-Me)Phenyl |
| NHCO(4) | 0 | Atomic bond | (2,6-Cl)Phenyl |
| NHCO(4) | 0 | Atomic bond | (3-Cl)Phenyl |
| NHCO(4) | 1 | Atomic bond | Phenyl |
| NHC(═S)NH(4) | 0 | Atomic bond | Phenyl |
| NHCO(4) | 0 | Atomic bond | (2,3-OMe)Phenyl |
| NHCO(4) | 0 | Atomic bond | (2-OMe)Phenyl |
| NHCO(4) | 1 | Atomic bond | (2-Cl)Phenyl |
| NHCO(4) | 0 | Atomic bond | (2,3-Me)Phenyl |
| NHCO(4) | 0 | Atomic bond | (2,5-Me)Phenyl |
| NHCO(4) | 0 | Atomic bond | (2-Cl,5-Br)Phenyl |

TABLE 9

| B^{Ba} (substitution | | | |
|---|---|---|---|
| position) | n^{B} | E^{Ba} | G^{Ba} |
| NHCO(4) | 0 | Atomic bond | (2,4-Cl)Phenyl |
| NHCO(4) | 0 | Atomic bond | (2-OH)Phenyl |
| NHCO(4) | 0 | Atomic bond | (2,3-OH)Phenyl |
| NHC(═O)NH(4) | 0 | Atomic bond | Phenyl |
| NHCO(4) | 1 | Atomic bond | (2,6-Cl)Phenyl |
| NHCO(4) | 1 | Atomic bond | (2-OMe)Phenyl |
| NHCO(4) | 1 | Atomic bond | (2-OH)Phenyl |
| NHC(═S)NH(4) | 0 | Atomic bond | (2-Cl)Phenyl |
| NHCO(4) | 0 | Atomic bond | (3-CF₃)Phenyl |
| NHCO(4) | 1 | Atomic bond | (2-CF₃)Phenyl |
| NHC(═O)NH(4) | 0 | Atomic bond | (2-Cl)Phenyl |
| NHCO(4) | 0 | Atomic bond | (2-Cl,3-OMe)Phenyl |
| NHCO(4) | 2 | Atomic bond | Phenyl |
| NHCO(4) | 0 | Atomic bond | 3-indolyl |
| NHCO(4) | 0 | Atomic bond | (2-Cl,3-OH)Phenyl |
| NHCO(4) | 1 | O | Phenyl |

TABLE 10

| B^{Ba} (substitution | | | |
|---|---|---|---|
| position) | n^{B} | E^{Ba} | G^{Ba} |
| NHCO(4) | 1 | Atomic bond | (2-Cl,4-OMe)Phenyl |
| NHCO(4) | 0 | Atomic bond | (1-Me)imidazol 2-yl |
| NHCO(4) | 1 | Atomic bond | (2,4-Cl)Phenyl |
| NHCO(4) | 1 | Atomic bond | (2-Cl,4-OH)Phenyl |
| NHCO(4) | 1 | Atomic bond | pyridin 3-yl |
| NHCO(4) | 0 | Atomic bond | Benzimidazol 2-yl |
| NHCO(4) | 0 | Atomic bond | (2-Cl)Phenyl |
| NHCO(4) | 0 | Atomic bond | (2-Br)Phenyl |
| NHCO(4) | 0 | Atomic bond | (2-I)Phenyl |
| NHCO(4) | 1 | Atomic bond | (2-Me)Phenyl |
| NHCO(4) | 0 | Atomic bond | quinoxalin 2-yl |
| NHCO(4) | 0 | Atomic bond | (5-Me)thiophen 2-yl |
| NHCO(3) | 1 | Atomic bond | (2-Cl)Phenyl |
| NHCO(4) | 0 | Atomic bond | (2,4,6-Me)Phenyl |
| NHCO(4) | 0 | Atomic bond | (2-Et)Phenyl |
| NHC(═S)NH(4) | 0 | Atomic bond | (2-Me)Phenyl |

TABLE 11

| B^{Ba} (substitution | | | |
|---|---|---|---|
| position) | n^{B} | E^{Ba} | G^{Ba} |
| NHCO(4) | 0 | Atomic bond | (4-NMe₂)Phenyl |
| NHCO(4) | 1 | O | (2,4-Cl)Phenyl |
| NHCO(4) | 1 | O | (2-Me)Phenyl |
| NHCO(4) | 0 | Atomic bond | (2-Ac)Phenyl |
| NHCO(4) | 0 | Atomic bond | (2-tBu)Phenyl |
| NHCO(3) | 0 | Atomic bond | (2-I)Phenyl |
| NHCO(4) | 0 | Atomic bond | (1-Me)piperidin 4-yl |
| NHCO(4) | 0 | Atomic bond | benzofuran 2-yl |
| NHCO(4) | 0 | Atomic bond | (1-Me)indol 3-yl |
| NHCO(4) | 0 | Atomic bond | (2-allyl)Phenyl |
| NHCO(4) | 0 | Atomic bond | (2-nPr)Phenyl |
| NHCO(4) | 0 | Atomic bond | (2-iPrO)Phenyl |
| NHCO(4) | 0 | Atomic bond | 3-Me thiophen 2-yl |
| NHCO(4) | 1 | O | (2-Me,3-Cl)Phenyl |
| NHCO(4) | 0 | Atomic bond | (2-CF₃,4-F)Phenyl |
| NHCO(4) | 0 | Atomic bond | (2-OMe,4-F)Phenyl |

TABLE 12

| B^{Ba} (substitution | | | |
|---|---|---|---|
| position) | n^{B} | E^{Ba} | G^{Ba} |
| NHCO(4) | 0 | Atomic bond | (2-OH,4-F)Phenyl |
| NHCO(3) | 1 | Atomic bond | (2-I)Phenyl |
| NHCO(4) | 0 | Atomic bond | (3-NMe₂)Phenyl |
| NHCO(4) | 0 | Atomic bond | (2-OMe,4-I)Phenyl |
| NHCO(4) | 0 | Atomic bond | (2-OMe,6-F)Phenyl |
| NHCO(4) | 0 | Atomic bond | (2-OH,4-I)Phenyl |
| NHCO(4) | 0 | Atomic bond | (2-OH,6-F)Phenyl |
| NHCO(4) | 0 | Atomic bond | (2-F)Phenyl |
| NHCO(4) | 0 | Atomic bond | (2-NMe₂)Phenyl |
| NHCO(4) | 0 | Atomic bond | (2-OMe,6-Me)Phenyl |
| NHCO(4) | 0 | Atomic bond | (2-OH,6-Me)Phenyl |
| NHCO(4) | 2 | Atomic bond | (2-Me)Phenyl |
| CONH(4) | 0 | Atomic bond | Phenyl |
| CONH(4) | 1 | Atomic bond | Phenyl |
| NHCO(4) | 2 | Atomic bond | (2-Cl)Phenyl |
| CONH(4) | 1 | Atomic bond | (2-Cl)Phenyl |

TABLE 13

| B^{Ba} (substitution | | | |
|---|---|---|---|
| position) | n^{B} | E^{Ba} | G^{Ba} |
| CONH(4) | 0 | Atomic bond | (2-Cl)Phenyl |
| NHCO(4) | 0 | Atomic bond | (5-Br,2,3- |
| | | | methylenedioxy)Phenyl |
| NHCO(4) | 0 | Atomic bond | (2-OMe,6-Br)Phenyl |
| NHCO(4) | 0 | Atomic bond | (2-OH,6-Br)Phenyl |
| NHCO(4) | 0 | Atomic bond | (2-OMe,6-Cl)Phenyl |
| NHCO(4) | 0 | Atomic bond | (2-OH,6-Cl)Phenyl |
| NHCO(4) | 0 | Atomic bond | (2-OH,6-OMe)Phenyl |
| NHCO(4) | 0 | Atomic bond | (2-OMe,6-CF₃)Phenyl |
| NHCO(4) | 0 | Atomic bond | (2-OH,6-CF₃)Phenyl |
| NHCO(4) | 0 | Atomic bond | (2-Cl,5-SMe)Phenyl |
| NHCO(4) | 0 | Atomic bond | (2-SMe)Phenyl |
| NHCO(4) | 0 | Atomic bond | (3-SMe)Phenyl |
| NHCO(4) | 0 | Atomic bond | (2-OMe,6-Et)Phenyl |
| NHCO(4) | 0 | Atomic bond | (3-SO₂Me)Phenyl |
| NHCO(4) | 0 | Atomic bond | (2-OH,6-Et)Phenyl |
| NHCO(4) | 0 | Atomic bond | (3-S(═O)Me)Phenyl |

TABLE 14

| B^{Ba} (substitution | | | |
|---|---|---|---|
| position) | n^{B} | E^{Ba} | G^{Ba} |
| NHCO(4) | 0 | Atomic bond | (2-Cl,5-S(═O)Me)Phenyl |
| NHCO(4) | 0 | Atomic bond | (2-S(═O)Me)Phenyl |
| NHCO(4) | 0 | Atomic bond | (3-Cl)pyridin 2-yl |
| NHCO(4) | 0 | Atomic bond | (2-OMe,3-Cl)Phenyl |
| NHCO(4) | 0 | Atomic bond | (3-Me)pyridin 2-yl |
| NHCO(4) | 0 | Atomic bond | (2-OH,3-Cl)Phenyl |
| NHCO(4) | 0 | Atomic bond | (3-OH)pyridin 2-yl |
| NHCO(4) | 0 | Atomic bond | (3-Vinyl)pyridin 2-yl |
| NHCO(4) | 0 | Atomic bond | (2-Et)pyridin 2-yl |
| NHSO₂(4) | 0 | Atomic bond | (2-NO₂)Phenyl |
| NHSO₂(4) | 0 | Atomic bond | Phenyl |
| NHSO₂(4) | 0 | Atomic bond | (3-Br)Phenyl |
| NHSO₂(4) | 0 | Atomic bond | (3-OMe)Phenyl |
| NHSO₂(3) | 0 | Atomic bond | (2-NO₂)Phenyl |
| NMeSO₂(3) | 0 | Atomic bond | (2-NO₂)Phenyl |
| NHSO₂(3) | 0 | Atomic bond | naphthalen 2-yl |

TABLE 15

| B^{Ba} (substitution | | | |
|---|---|---|---|
| position) | n^{B} | E^{Ba} | G^{Ba} |
| NHSO₂(3) | 0 | Atomic bond | naphthalen 1-yl |
| NHSO₂(4) | 0 | Atomic bond | Cyclohexyl |
| NHSO₂(4) | 0 | Atomic bond | pyridin 3-yl |
| NHSO₂(4) | 0 | Atomic bond | (4-iPr)Phenyl |
| NHSO₂(4) | 1 | Atomic bond | Phenyl |
| NHSO₂(4) | 0 | Atomic bond | thiophen 2-yl |
| NHSO₂(4) | 0 | Atomic bond | naphthalen 2-yl |
| NBnSO₂(4) | 0 | Atomic bond | (2-NO₂)Phenyl |
| NMeSO₂(4) | 0 | Atomic bond | (3-Br)Phenyl |
| NMeSO₂(4) | 0 | Atomic bond | (2-NO₂)Phenyl |
| N(CH₂CH₂OH)SO₂(4) | 0 | Atomic bond | (2-NO₂)Phenyl |
| NHSO₂(4) | 1 | Atomic bond | (2-Cl)Phenyl |
| NHSO₂(4) | 1 | Atomic bond | (3-Br)Phenyl |
| NHSO₂(4) | 0 | Atomic bond | (2-CF₃)Phenyl |
| NHSO₂(4) | 1 | Atomic bond | (2-Br)Phenyl |
| NHSO₂(4) | 1 | Atomic bond | (2-Me)Phenyl |

TABLE 16

| B^{Ba} (substitution | | | |
|---|---|---|---|
| position) | n^{B} | E^{Ba} | G^{Ba} |
| NHSO₂(4) | 1 | Atomic bond | (2-NO₂)Phenyl |
| NHSO₂(4) | 2 | Atomic bond | Phenyl |
| NHSO₂(4) | 1 | Atomic bond | (4-Cl)Phenyl |
| NMeSO₂(4) | 1 | Atomic bond | (2-CF₃)Phenyl |
| NMeSO₂(4) | 1 | Atomic bond | (2-Et)Phenyl |
| NMeSO₂(4) | 1 | Atomic bond | (2,3-Me)Phenyl |
| NMeSO₂(4) | 2 | Atomic bond | (2-Cl)Phenyl |
| NMeSO₂(4) | 1 | Atomic bond | (2-NO₂)Phenyl |
| NMeSO₂(4) | 1 | Atomic bond | (2-NH₂)Phenyl |
| NMeSO₂(4) | 1 | Atomic bond | (2-NMe₂)Phenyl |

TABLE 17

| B^{Ba} (substitution | | | |
|---|---|---|---|
| position) | n^{B} | E^{Ba} | G^{Ba} |
| NHCO(4) | 0 | Atomic bond | pyridin 4-y1 |
| NHCO(4) | 1 | O | pyridin 3-yl |
| NHCO(4) | 0 | Atomic bond | pyridin 3-y1 |
| NHCO(4) | 0 | Atomic bond | (2-Me)pyridin 3-yl |
| NHCO(4) | 0 | Atomic bond | (2-Cl)pyridin 3-yl |
| NHCO(4) | 1 | O | pyridin 2-y1 |
| NHCO(4) | 0 | Atomic bond | (4-CF₃)pyridin 3-yl |
| NHCO(4) | 0 | Atomic bond | (2-iPr)Phenyl |

### <Typical Compound Example B-200>

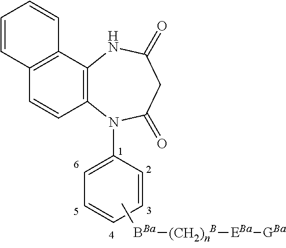

(B^{Ba} (substitution position), n^{B}, E^{Ba}, and G^{Ba} in the formula are indicated in Tables 18 and 19)

TABLE 18

| B^{Ba} (substitution | | | |
|---|---|---|---|
| position) | n^{B} | E^{Ba} | G^{Ba} |
| NHCO(4) | 0 | Atomic bond | Cyclohexyl |
| NHCO(4) | 0 | Atomic bond | (6-Me)pyridin-2-y1 |
| NHCO(4) | 0 | Atomic bond | (2-Me)pyridin-3-yl |
| NHCO(4) | 0 | Atomic bond | (2-OMe,3-Me)Phenyl |
| NHCO(4) | 0 | Atomic bond | (2,3-Cl)Phenyl |
| NHCO(4) | 0 | Atomic bond | (2-OH,3-Me)Phenyl |
| NHCO(4) | 0 | Atomic bond | (2-I)Phenyl |
| NHCO(4) | 1 | Atomic bond | (1-Me)pyrrol 2-yl |
| NHCO(4) | 1 | Atomic bond | (2-tBu)Phenyl |
| NHCO(4) | 0 | Atomic bond | (2-Isopropenyl)phenyl |
| NHCO(4) | 0 | Atomic bond | (2-iPr)Phenyl |
| NHCO(4) | 1 | Atomic bond | morpholin 2-yl |
| NHCO(4) | 0 | Atomic bond | (2-Cl)pyridin 2-yl |

TABLE 19

| B^{Ba} (substitution | | | |
|---|---|---|---|
| position) | n^{B} | E^{Ba} | G^{Ba} |
| NHSO₂(4) | 0 | Atomic bond | (2-NO₂)Phenyl |
| NMeSO₂(4) | 0 | Atomic bond | (2-NO₂)Phenyl |
| SO₂NH(4) | 0 | Atomic bond | Phenyl |
| OSO₂(4) | 0 | Atomic bond | (3-Br)Phenyl |
| NHSO₂(4) | 1 | Atomic bond | (2-Cl)Phenyl |
| NHSO₂(4) | 0 | Atomic bond | (3-Br)Phenyl |
| NHSO₂(4) | 0 | Atomic bond | (3-OMe)Phenyl |
| NHSO₂(4) | 1 | Atomic bond | (2,3-Cl)Phenyl |
| NHSO₂(4) | 1 | Atomic bond | (2,6-Cl)Phenyl |
| NHSO₂(4) | 1 | Atomic bond | (2-I)Phenyl |
| NMeSO₂(4) | 1 | Atomic bond | (2-Cl)Phenyl |

### <Typical Compound Example B-300>

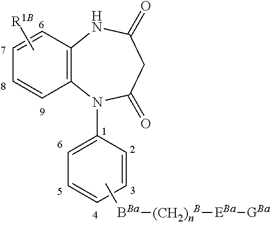

(R^{1B}, B^{Ba} (substitution position), n^{B}, E^{Ba}, and G^{Ba} in the formula are indicated in Table 20)

TABLE 20

| | B^{Ba} | | | |
|---|---|---|---|---|
| | (substitution | | | |
| R^{1B} | position) | n^{B} | E^{Ba} | G^{Ba} |
| 7-OMe | NHCO(4) | 0 | Atomic bond | (2,3-Me)Phenyl |
| 7-OH | NHCO(4) | 0 | Atomic bond | (2,3-Me)Phenyl |
| 6-Me | NHCO(4) | 0 | Atomic bond | (2,3-Me)Phenyl |
| 6,7-Me | NHCO(4) | 0 | Atomic bond | (2-I)Phenyl |
| 6-Et | NHCO(4) | 0 | Atomic bond | (2-I)Phenyl |
| 7-Ph | NHCO(4) | 0 | Atomic bond | (2-Isopropyl))Phenyl |
| 7-(Pyridin-3yl) | NHCO(4) | 0 | Atomic bond | (2-Isopropyl))Phenyl |
| 7-(Pyridin-2yl) | NHCO(4) | 0 | Atomic bond | (2-Isopropyl)Phenyl |
| 7-Cl | NHSO₂(4) | 0 | Atomic bond | (2-Isopropyl)Phenyl |
| 7-Br | NHSO₂(4) | 0 | Atomic bond | (2-Isopropyl)Phenyl |
| 7-CF₃ | NHSO₂(4) | 0 | Atomic bond | (2-Isopropyl)Phenyl |
| H | NHSO₂(4) | 0 | Atomic bond | (2-Isopropyl)Phenyl |
| 6-Me,7-Br | NHSO₂(4) | 0 | Atomic bond | (2-Isopropyl)Phenyl |
| 7-OMe | NHSO₂(4) | 1 | Atomic bond | (2-Cl)Phenyl |
| 7-OH | NHSO₂(4) | 1 | Atomic bond | (2-Cl)Phenyl |
| 6-Me | NHSO₂(4) | 1 | Atomic bond | (2-Cl)Phenyl |

### <Typical Compound Example B-400>

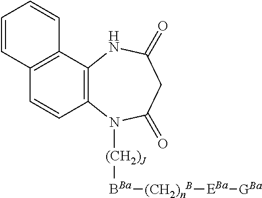

(B^{Ba} (substitution position), n^{B}, E^{Ba}, and G^{Ba} in the formula are indicated in Table 21)

TABLE 21

| B^{Ba} (substitution | | | |
|---|---|---|---|
| position | n^{B} | E^{Ba} | G^{Ba} |
| NHCO | 0 | Atomic bond | (2-Cl,3-OMe)Phenyl |
| NHCO | 0 | Atomic bond | (2-I)Phenyl |
| NHSO₂ | 1 | Atomic bond | (2-Cl)Phenyl |
| NHSO₂ | 1 | Atomic bond | (2-Cl)Phenyl |

### <Typical Compound Example B-500>

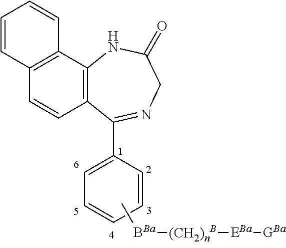

(B^{Ba} (substitution position), n^{B}, E^{Ba}, and G^{Ba} in the formula are indicated in Table 22)

TABLE 22

| B^{Ba} (substitution | | | |
|---|---|---|---|
| position) | n^{B} | E^{Ba} | G^{Ba} |
| NHCO(4) | 0 | Atomic bond | (2-Cl,3-OMe)Phenyl |
| NHCO(4) | 0 | Atomic bond | (2-Cl,3-OH)Phenyl |
| NHCO(4) | 0 | Atomic bond | (2-tBu)Phenyl |
| NHCO(4) | 0 | Atomic bond | (2-Cl,6-OMe)Phenyl |
| NHCO(4) | 0 | Atomic bond | (2-Cl,6-OH)Phenyl |
| NHSO2(3) | 0 | Atomic bond | Phenyl |
| NHSO2(4) | 0 | Atomic bond | (2-Cl)Phenyl |

### <Typical Compound Example B-600>

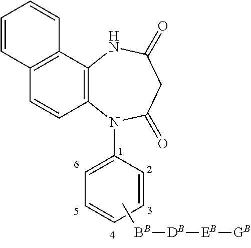

(B^{B} (substitution position), D^{B}, E^{B}, and G^{B} in the formula are indicated in Table 23)

TABLE 23

| B^{Ba} (substitution | | | |
|---|---|---|---|
| position | D^{B} | E^{B} | G^{B} |
| NHCO(4) | C(Me)H | Atomic bond | Phenyl |
| NHCO(4) | C(Me)₂ | Atomic bond | Phenyl |
| NHCO(4) | CH═CH | Atomic bond | Phenyl |
| NHCO(4) | C(Me)H | O | Phenyl |
| NHCO(4) | C(Me)₂ | O | Phenyl |
| NHCO(4) | CH═CH | Atomic bond | (2-Me)Phenyl |
| NHCO(4) | CH═CH | Atomic bond | (2-Cl)Phenyl |

### <Typical Compound Example B-700>

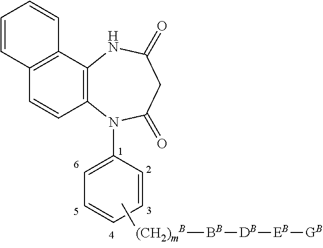

(m^{B} (substitution position), B^{B}, D^{B}, E^{B}, and G^{B} in the formula are indicated in Table 24)

TABLE 24

| m^{B} (substitution | | | | |
|---|---|---|---|---|
| position) | B^{B} | D^{B} | E^{B} | G^{B} |
| 1(4) | NHCO | Atomic bond | Atomic bond | Phenyl |
| 1(4) | NHCO | Atomic bond | Atomic bond | (2-Cl)Phenyl |
| 1(4) | NHSO₂ | CH₂ | Atomic bond | (2-Cl)Phenyl |

### <Typical Compound Example B-800>

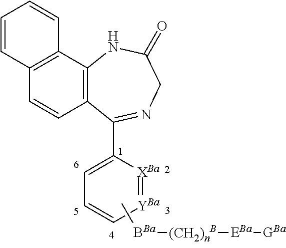

(X^{Ba}, Y^{Ba}, B^{Ba} (substitution position), n^{B}, E^{Ba}, and G^{Ba} in the formula are indicated in Table 25)

TABLE 25

| | | B^{Ba} | | | |
|---|---|---|---|---|---|
| | | (substitution | | | |
| X^{Ba} | Y^{Ba} | position) | n^{B} | E^{Ba} | G^{Ba} |
| CH | C—F | NHCO(4) | 0 | Atomic bond | (2,3-Me)Phenyl |
| CH | C—OH | NHCO(4) | 0 | Atomic bond | (2,3-Me)Phenyl |
| CH | C—F | NHCO(4) | 0 | Atomic bond | (2-I)Phenyl |
| CH | N | NHCO(4) | 0 | Atomic bond | (2-I)Phenyl |
| CH | N | NHCO(4) | 0 | Atomic bond | Phenyl |
| N | CH | NHCO(4) | 0 | Atomic bond | (2-I)Phenyl |
| CH | N | NHCO(4) | 0 | Atomic bond | (2-Cl)Phenyl |
| CH | N | NHCO(4) | 0 | Atomic bond | (2-OH)Phenyl |
| CH | N | NHC(═O)NH(4) | 0 | Atomic bond | (2-OH)Phenyl |
| CH | N | NHCO(4) | 0 | Atomic bond | (2-OH,6-Me)Phenyl |
| CH | N | NHCO(4) | 0 | Atomic bond | (2-OH,6-Cl)Phenyl |
| CH | N | NHCO(3) | 0 | Atomic bond | (2-OH,6-Cl)Phenyl |
| CH | N | NHCO(4) | 0 | Atomic bond | (2-Cl)pyridin 2-yl |
| CH | N | NHCO(4) | 1 | Atomic bond | (2-Cl)pyridin 2-yl |
| CH | N | NHCO(4) | 0 | Atomic bond | (2-Me)pyridin 2-yl |
| CH | C—OMe | NHSO₂(4) | 1 | Atomic bond | (2-Cl)Phenyl |
| CH | C—OH | NHSO₂(4) | 1 | Atomic bond | (2-Cl)Phenyl |

### <Typical Compound Example B-900>

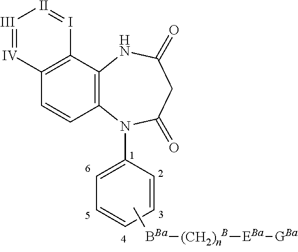

(I=II-III=IV, B^{Ba} (substitution position), n^{B}, E^{Ba}, and G^{Ba} in the formula are indicated in Table 26)

TABLE 26

| | B^{Ba} | | | |
|---|---|---|---|---|
| | (substitution | | | |
| I = II-III = IV | position) | n^{B} | E^{Ba} | G^{Ba} |
| N═CH—CH=CH | NHCO(4) | 0 | Atomic bond | (2-I)Phenyl |
| CH═N—CH=CH | NHCO(4) | 0 | Atomic bond | (2-I)Phenyl |
| CH═CH—N═CH | NHCO(4) | 0 | Atomic bond | (2-I)Phenyl |
| CH═CH—CH═N | NHCO(4) | 0 | Atomic bond | (2-I)Phenyl |
| N═CH—CH═CH | NHCO(4) | 1 | O | Phenyl |
| N═CH—CH═CH | NHCO(3) | 0 | Atomic bond | (2-I)Phenyl |
| N═CH—CH═CH | NHCO(4) | 0 | Atomic bond | (2-Cl)Phenyl |
| N═CH—CH═CH | NHCO(4) | 0 | Atomic bond | (2-OH)Phenyl |
| N═CH—CH═CH | NHC(═O)NH(4) | 0 | Atomic bond | (2-OH)Phenyl |
| N═CH—CH═CH | NHCO(4) | 1 | O | (2-OH,6-Me)Phenyl |
| N═CH—CH═CH | NHCO(4) | 0 | Atomic bond | (2-OH,6-Cl)Phenyl |
| N═CH—CH═CH | NHCO(3) | 0 | Atomic bond | (2-OH,6-Cl)Phenyl |
| N═CH—CH═CH | NHCO(4) | 0 | Atomic bond | (2-Cl)pyridin 2-yl |
| N═CH—CH═CH | NHCO(4) | 1 | Atomic bond | (2-Cl)pyridin 2-yl |
| N═CH—CH═CH | NHCO(4) | 0 | Atomic bond | (2-Me)pyridin 2-yl |
| CH═CH—N═CH | NHCO(4) | 0 | Atomic bond | (2-Cl)pyridin 3-yl |

### <Typical Compound Example B-1000>

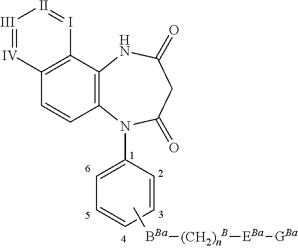

(I-II-III-IV, B^{Ba} (substitution position), n^{B}, E^{Ba}, and G^{Ba} in the formula are indicated in Table 27)

TABLE 27

| | B^{Ba} | | | |
|---|---|---|---|---|
| | (substitution | | | |
| I-II-III-IV | position) | n^{B} | E^{Ba} | G^{Ba} |
| NH—CH₂—CH₂—CH₂ | NHCO(4) | 0 | Atomic bond | (2-I)Phenyl |
| CH₂—NH—CH₂—CH₂ | NHCO(4) | 0 | Atomic bond | (2-I)Phenyl |
| CH₂—CH₂—NH—CH₂ | NHCO(4) | 0 | Atomic bond | (2-I)Phenyl |
| CH₂—CH₂—CH₂—NH | NHCO(4) | 0 | Atomic bond | (2-I)Phenyl |
| CH₂—CH₂—NH—CH₂ | NHCO(4) | 1 | O | Phenyl |
| CH₂—CH₂—NH—CH₂ | NHCO(3) | 0 | Atomic bond | (2-I)Phenyl |
| CH₂—CH₂—NH—CH₂ | NHCO(4) | 0 | Atomic bond | (2-Cl)Phenyl |
| CH₂—CH₂—NH—CH₂ | NHCO(4) | 0 | Atomic bond | (2-Cl)pyridin 3-yl |
| CH₂—CH₂—NH—CH₂ | NHCO(4) | 0 | Atomic bond | (2-OH)Phenyl |
| CH₂—CH₂—NH—CH₂ | NHC(═O)NH(4) | 0 | Atomic bond | (2-OH)Phenyl |
| CH₂—CH₂—NH—CH₂ | NHCO(4) | 1 | O | (2-OH,6-Me)Phenyl |
| CH₂—CH₂—NH—CH₂ | NHCO(4) | 0 | Atomic bond | (2-OH,6-Cl)Phenyl |
| CH₂—CH₂—NH—CH₂ | NHCO(3) | 0 | Atomic bond | (2-OH,6-Cl)Phenyl |
| CH₂—CH₂—NH—CH₂ | NHCO(4) | 0 | Atomic bond | (2-Cl)pyridin 2-yl |
| CH₂—CH₂—NH—CH₂ | NHCO(4) | 1 | Atomic bond | (2-Cl)pyridin 2-yl |
| CH₂—CH₂—NH—CH₂ | NHCO(4) | 0 | Atomic bond | (2-Me)pyridin 2-yl |
| CH₂—CH₂—NH—CH₂ | NHCO(4) | 0 | Atomic bond | (2-Cl)pyridin 3-yl |

### <Typical Compound Example B-1100>

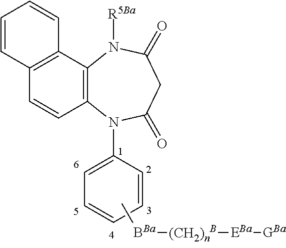

(R^{5Ba}, B^{Ba} (substitution position), n^{B}, E^{Ba}, and G^{Ba} in the formula are indicated in Table 28)

TABLE 28

| | B^{Ba} (substitution | | | |
|---|---|---|---|---|
| R^{5Ba} | position | n^{B} | E^{Ba} | G^{Ba} |
| Bn | NBnSO₂(4) | 0 | Atomic bond | (2-NO₂)Phenyl |
| Me | NBnSO₂(4) | 0 | Atomic bond | (2-NO₂)Phenyl |
| Et | NBnSO₂(4) | 0 | Atomic bond | (2-NO₂)Phenyl |

Since the compounds represented by the general formula (A) are disclosed in WO 2010/093061 A, all of the compounds can be easily obtained by referring to this international publication. The disclosure of this international publication is incorporated herein by reference in its entirety.

Since the compounds represented by the general formulas (BI) and (BII) are disclosed in WO 2013/105608 A, all of the compounds can be easily obtained by referring to this international publication. The disclosure of this international publication is incorporated herein by reference in its entirety.

Furthermore, WO 2010/093061 A and WO 2013/105608 A disclose that the compounds represented by the general formulas (A) to (BII) have a P2X4 receptor antagonistic action.

Specific examples of a preferable compound included in the compounds represented by the general formulas (A) to (BII) or a pharmaceutically acceptable salt thereof are shown below, however, the compound or a pharmaceutically acceptable salt thereof that can be used as the active ingredient of the medicament of the present invention is not limited thereto.
(Compound A1) 5-[3-(1H-tetrazol-5-yl)phenyl]-1H-naphtho[1,2-b][1,4]diazepine-2,4(3H,5H)-dione;
(Compound A2) 5-[3-(1H-tetrazol-5-yl)phenyl]-1H-naphtho[1,2-b][1,4]diazepine-2,4(3H,5H)-dione sodium salt;
(Compound A3) 5-[3-(1H-tetrazol-5-yl)phenyl]-1H-naphtho[1,2-b][1,4]diazepine-2,4(3H,5H)-dione potassium salt;
(Compound A4) 5-[4-(1H-tetrazol-5-yl)phenyl]-1H-naphtho[1,2-b][1,4]diazepine-2,4(3H,5H)-dione;
(Compound A5) 5-[4-(1H-tetrazol-5-yl)phenyl]-1H-naphtho[1,2-b][1,4]diazepine-2,4(3H,5H)-dione sodium salt;
(Compound A6) 1-methyl-5-[3-(1H-tetrazol-5-yl)phenyl]-1H-naphtho[1,2-b][1,4]diazepine-2,4(3H,5H)-dione;
(Compound A7) 1,3-dimethyl-5-[3-(1H-tetrazol-5-yl)phenyl]-1H-naphtho[1,2-b][1,4]diazepine-2,4(3H,5H)-dione;
(Compound A8) 5-[2-chloro-5-(1H-tetrazol-5-yl)phenyl]-1H-naphtho[1,2-b][1,4]diazepine-2,4(3H,5H)-dione;
(Compound A9) 5-[2-chloro-5-(1H-tetrazol-5-yl)phenyl]-1H-naphtho[1,2-b][1,4]diazepine-2,4(3H,5H)-dione sodium salt;
(Compound A10) 5-[2-methyl-5-(1H-tetrazol-5-yl)phenyl]-1H-naphtho[1,2-b][1,4]diazepine-2,4(3H,5H)-dione;
(Compound A11) 5-[2-methyl-5-(1H-tetrazol-5-yl)phenyl]-1H-naphtho[1,2-b][1,4]diazepine-2,4(3H,5H)-dione sodium salt;
(Compound A12) 5-[2-bromo-5-(1H-tetrazol-5-yl)phenyl]-1H-naphtho[1,2-b][1,4]diazepine-2,4(3H,5H)-dione;
(Compound A13) 5-[3-(2-methyl-2H-tetrazol-5-yl)phenyl]-1H-naphtho[1,2-b][1,4]diazepine-2,4(3H,5H)-dione;
(Compound A14) 5-[3-(1-methyl-1H-tetrazol-5-yl)phenyl]-1H-naphtho[1,2-b][1,4]diazepine-2,4(3H,5H)-dione;
(Compound A15) 5-[3-(5-oxo-4H-[1,2,4]oxadiazol-3-yl)phenyl]-1H-naphtho[1,2-b][1,4]diazepine-2,4(3H,5H)-dione;
(Compound A16) 5-[3-(5-thioxo-4H-[1,2,4]oxadiazol-3-yl)phenyl]-1H-naphtho[1,2-b][1,4]diazepine-2,4(3H,5H)-dione;
(Compound A17) 5-[3-(5-thioxo-4H-[1,2,4]oxadiazol-3-yl)phenyl]-1H-naphtho[1,2-b][1,4]diazepine-2,4(3H,5H)-dione sodium salt;
(Compound A18) 5-[3-(oxazol-2-yl)phenyl]-1H-naphtho[1,2-b][1,4]diazepine-2,4(3H,5H)-dione;
(Compound A19) 5-[3-(1H-pyrazol-4-yl)phenyl]-1H-naphtho[1,2-b][1,4]diazepine-2,4(3H,5H)-dione;
(Compound A20) 5-[4-fluoro-3-(1H-tetrazol-5-yl)phenyl]-1H-naphtho[1,2-b][1,4]diazepine-2,4(3H,5H)-dione;
(Compound A21) 5-[4-fluoro-3-(1H-tetrazol-5-yl)phenyl]-1H-naphtho[1,2-b][1,4]diazepine-2,4(3H,5H)-dione sodium salt;
(Compound B1) 5-(4-benzoylaminophenyl)-1H-naphtho[1,2-b][1,4]diazepine-2,4(3H,5H)-dione;
(Compound B2) 5-[4-[2-(trifluoromethyl)benzoyl]aminophenyl]-1H-naphtho[1,2-b][1,4]diazepine-2,4(3H,5H)-dione;
(Compound B3) 5-[4-(3-bromobenzoyl)aminophenyl]-1H-naphtho[1,2-b][1,4]diazepine-2,4(3H,5H)-dione;
(Compound B4) 5-[4-[4-(trifluoromethyl)benzoyl]aminophenyl]-1H-naphtho[1,2-b][1,4]diazepine-2,4(3H,5H)-dione;
(Compound B5) 5-[4-(2-methylbenzoyl)aminophenyl]-1H-naphtho[1,2-b][1,4]diazepine-2,4(3H,5H)-dione;
(Compound B6) 5-[4-(2,6-dimethylbenzoylamino)phenyl]-1H-naphtho[1,2-b][1,4]diazepine-2,4(3H,5H)-dione;
(Compound B7) 5-[4-(2,6-dichlorobenzoyl)aminophenyl]-1H-naphtho[1,2-b][1,4]diazepine-2,4(3H,5H)-dione;
(Compound B8) 5-[4-(3-chlorobenzoyl)aminophenyl]-1H-naphtho[1,2-b][1,4]diazepine-2,4(3H,5H)-dione;
(Compound B9) 5-[4-(2-phenylacetylamino)phenyl]-1H-naphtho[1,2-b][1,4]diazepine-2,4(3H,5H)-dione;
(Compound B10) 1-[4-(2,4-dioxo-1,2,3,4-tetrahydronaphtho[1,2-b][1,4]diazepin-5-yl)phenyl]-3-phenylthiourea;
(Compound B11) 5-[4-(2,3-dimethoxybenzoylamino)phenyl]-1H-naphtho[1,2-b][1,4]diazepine-2,4(3H,5H)-dione;
(Compound B12) 5-[4-(2-methoxybenzoylamino)phenyl]-1H-naphtho[1,2-b][1,4]diazepine-2,4(3H,5H)-dione;
(Compound B13) 5-[4-[(2-chlorophenylacetyl)amino]phenyl]-1H-naphtho[1,2-b][1,4]diazepine-2,4(3H,5H)-dione;
(Compound B14) 5-[4-(2,3-dimethylbenzoylamino)phenyl]-1H-naphtho[1,2-b][1,4]diazepine-2,4(3H,5H)-dione;
(Compound B15) 5-[4-(2,5-dimethylbenzoylamino)phenyl]-1H-naphtho[1,2-b][1,4]diazepine-2,4(3H,5H)-dione;
(Compound B16) 5-[4-(5-bromo-2-chlorobenzoylamino)phenyl]-1H-naphtho[1,2-b][1,4]diazepine-2,4(3H,5H)-dione;
(Compound B17) 5-[4-(2,4-dichlorobenzoylamino)phenyl]-1H-naphtho[1,2-b][1,4]diazepine-2,4(3H,5H)-dione;
(Compound B18) 5-[4-(2-hydroxybenzoylamino)phenyl]-1H-naphtho[1,2-b][1,4]diazepine-2,4(3H,5H)-dione;
(Compound B19) 5-[4-(2,3-dihydroxybenzoylamino)phenyl]-1H-naphtho[1,2-b][1,4]diazepine-2,4(3H,5H)-dione;
(Compound B20) 1-[4-(2,4-dioxo-1,2,3,4-tetrahydronaphtho[1,2-b][1,4]diazepin-5-yl)phenyl]-3-phenylurea;
(Compound B21) 5-[4-[(2,6-dichlorophenylacetyl)amino]phenyl]-1H-naphtho[1,2-b][1,4]diazepine-2,4(3H,5H)-dione;
(Compound B22) 5-[4-[(2-methoxyphenylacetyl)amino]phenyl]-1H-naphtho[1,2-b][1,4]diazepine-2,4(3H,5H)-dione;
(Compound B23) 5-[4-[(2-hydroxyphenylacetyl)amino]phenyl]-1H-naphtho[1,2-b][1,4]diazepine-2,4(3H,5H)-dione;
(Compound B24) 1-(2-chlorophenyl)-3-[4-(2,4-dioxo-1,2,3,4-tetrahydronaphtho[1,2-b][1,4]diazepin-5-yl)phenyl]thiourea;
(Compound B25) 5-[4-[3-(trifluoromethyl)benzoylamino]phenyl]-1H-naphtho[1,2-b][1,4]diazepine-2,4(3H,5H)-dione;
(Compound B26) 5-[4-[2-[2-(trifluoromethyl)phenyl]acetylamino]phenyl]-1H-naphtho[1,2-b][1,4]diazepine-2,4(3H,5H)-dione;
(Compound B27) 1-(2-chlorophenyl)-3-[4-(2,4-dioxo-1,2,3,4-tetrahydronaphtho[1,2-b][1,4]diazepin-5-yl)phenyl]urea;
(Compound B28) 5-[4-[(2-phenylpropionyl)amino]phenyl]-1H-naphtho[1,2-b][1,4]diazepine-2,4(3H,5H)-dione;
(Compound B29) 5-[4-(2-chloro-3-methoxybenzoylamino)phenyl]-1H-naphtho[1,2-b][1,4]diazepine-2,4(3H,5H)-dione;
(Compound B30) 5-[4-(3-phenylpropionylamino)phenyl]-1H-naphtho[1,2-b][1,4]diazepine-2,4(3H,5H)-dione;
(Compound B31) 5-[4-[(1H-indole-3-carbonyl)amino]phenyl]-1H-naphtho[1,2-b][1,4]diazepine-2,4(3H,5H)-dione;
(Compound B32) 5-[4-(2-chloro-3-hydroxybenzoylamino)phenyl]-1H-naphtho[1,2-b][1,4]diazepine-2,4(3H,5H)-dione;
(Compound B33) 5-[4-[(2-methyl-2-phenylpropionyl)amino]phenyl]-1H-naphtho[1,2-b][1,4]diazepine-2,4(3H,5H)-dione;
(Compound B34) 5-[4-(2-phenoxyacetylamino)phenyl]-1H-naphtho[1,2-b][1,4]diazepine-2,4(3H,5H)-dione;
(Compound B35) 5-[4-[2-(2-chloro-4-methoxyphenyl)acetylamino]phenyl]-1H-naphtho[1,2-b][1,4]diazepine-2,4(3H,5H)-dione;
(Compound B36) 5-[4-[(1-methyl-1H-imidazole-2-carbonyl)amino]phenyl]-1H-naphtho[1,2-b][1,4]diazepine-2,4(3H,5H)-dione;
(Compound B37) 5-[4-[2-(2,4-dichlorophenyl)acetylamino]phenyl]-1H-naphtho[1,2-b][1,4]diazepine-2,4(3H,5H)-dione;
(Compound B38) 5-[4-[2-(2-chloro-4-hydroxyphenyl)acetylamino]phenyl]-1H-naphtho[1,2-b][1,4]diazepine-2,4(3H,5H)-dione;
(Compound B39) 5-[4-(3-phenylpropenylamino)phenyl]-1H-naphtho[1,2-b][1,4]diazepine-2,4(3H,5H)-dione;
(Compound B40) 5-[4-[(3-pyridylacetyl)amino]phenyl]-1H-naphtho[1,2-b][1,4]diazepine-2,4(3H,5H)-dione hydrochloride;
(Compound B41) 5-[4-(1H-benzimidazole-2-carbonylamino)phenyl]-1H-naphtho[1,2-b][1,4]diazepine-2,4(3H,5H)-dione;
(Compound B42) 1-[4-(2,3-dimethylbenzoylamino)phenyl]-7-methoxy-1H-1,5-benzodiazepine-2,4(3H,5H)-dione;
(Compound B43) 5-[4-[(benzoylamino)methyl]phenyl]-1H-naphtho[1,2-b][1,4]diazepine-2,4(3H,5H)-dione;
(Compound B44) 5-[4-[(2-chlorobenzoylamino)methyl]phenyl]-1H-naphtho[1,2-b][1,4]diazepine-2,4(3H,5H)-dione;
(Compound B45) 1-[4-(2,3-dimethylbenzoylamino)phenyl]-7-hydroxy-1H-1,5-benzodiazepine-2,4(3H,5H)-dione;
(Compound B46) 5-[4-(2-chlorobenzoylamino)phenyl]-1H-naphtho[1,2-b][1,4]diazepine-2,4(3H,5H)-dione;
(Compound B47) 5-[4-(2-bromobenzoylamino)phenyl]-1H-naphtho[1,2-b][1,4]diazepine-2,4(3H,5H)-dione;
(Compound B48) 5-[4-(2-iodobenzoylamino)phenyl]-1H-naphtho[1,2-b][1,4]diazepine-2,4(3H,5H)-dione;
(Compound B49) 5-[4-(2,3-dimethylbenzoylamino)-3-fluorophenyl]-1H-naphtho[1,2-b][1,4]diazepine-2,4(3H,5H)-dione;
(Compound B50) 5-[4-[2-(2-methylphenyl)acetylamino]phenyl]-1H-naphtho[1,2-b][1,4]diazepine-2,4(3H,5H)-dione;
(Compound B51) 5-[4-[(quinoxalin-2-yl)carbonylamino]phenyl]-1H-naphtho[1,2-b][1,4]diazepine-2,4(3H,5H)-dione;
(Compound B52) 5-[4-[(5-methylthiophen-2-yl)carbonylamino]phenyl]-1H-naphtho[1,2-b][1,4]diazepine-2,4(3H,5H)-dione;
(Compound B53) 5-[3-[(2-chlorophenylacetyl)amino]phenyl]-1H-naphtho[1,2-b][1,4]diazepine-2,4(3H,5H)-dione;
(Compound B54) 5-[4-[(2,4,6-trimethylbenzoyl)amino]phenyl]-1H-naphtho[1,2-b][1,4]diazepine-2,4(3H,5H)-dione;
(Compound B55) 5-[4-(cyclohexylcarbonylamino)phenyl]-1,2,3,4,8,9,10,11-octahydronaphtho[1,2-b][1,4]diazepine-2,4(3H,5H)-dione;
(Compound B56) 1-[4-(2,3-dimethylbenzoyl)aminophenyl]-6-methyl-1H-1,5-benzodiazepine-2,4(3H,5H)-dione;
(Compound B57) 5-[4-[(2-ethylbenzoyl)amino]phenyl]-1H-naphtho[1,2-b][1,4]diazepine-2,4(3H,5H)-dione;
(Compound B58) 5-[4-[(6-methylpyridin-2-yl)carbonylamino]phenyl]-1,2,3,4,8,9,10,11-octahydronaphtho[1,2-b][1,4]diazepine-2,4(3H,5H)-dione;
(Compound B59) 5-[4-[(2-methylpyridin-3-yl)carbonylamino]phenyl]-1,2,3,4,8,9,10,11-octahydronaphtho[1,2-b][1,4]diazepine-2,4(3H,5H)-dione;
(Compound B60) 1-[4-(2,4-dioxo-1,2,3,4-tetrahydronaphtho[1,2-b][1,4]diazepin-5-yl)phenyl]-3-(2-methylphenyl)thiourea;
(Compound B61) 5-[4-(2-methoxy-3-methylbenzoyl)aminophenyl]-1,2,3,4,8,9,10,11-octahydronaphtho[1,2-b][1,4]diazepine-2,4(3H,5H)-dione;
(Compound B62) 5-[4-(2,3-dichlorobenzoyl)aminophenyl]-1,2,3,4,8,9,10,11-octahydronaphtho[1,2-b][1,4]diazepine-2,4(3H,5H)-dione;
(Compound B63) 5-[4-(2,3-dimethylbenzoylamino)-3-hydroxyphenyl]-1H-naphtho[1,2-b][1,4]diazepine-2,4(3H,5H)-dione;
(Compound B64) 5-[4-(2-chloro-3-methoxybenzoylamino)phenyl]-1,3-dihydronaphtho[1,2-e]-1,4-diazepin-2-one;
(Compound B65) 5-[4-[(4-dimethylaminobenzoyl)amino]phenyl]-1H-naphtho[1,2-b][1,4]diazepine-2,4(3H,5H)-dione;
(Compound B66) 5-[4-[2-(2,4-dichlorophenoxy)acetylamino]phenyl]-1H-naphtho[1,2-b][1,4]diazepine-2,4(3H,5H)-dione;
(Compound B67) 5-[4-[2-(2-methylphenoxy)acetylamino]phenyl]-1H-naphtho[1,2-b][1,4]diazepine-2,4(3H,5H)-dione;
(Compound B68) N-[4-(2,4-dioxo-1,2,3,4-tetrahydronaphtho[1,2-b][1,4]diazepin-5-yl)butyl]-2-chloro-3-methoxybenzamide;
(Compound B69) 5-[4-(2-chloro-3-hydroxybenzoylamino)phenyl]-1,3-dihydronaphtho[1,2-e]-1,4-diazepin-2-one;
(Compound B70) 5-[4-(2-acetylbenzoylamino)phenyl]-1H-naphtho[1,2-b][1,4]diazepine-2,4(3H,5H)-dione;
(Compound B71) 5-[4-(2-tert-butylbenzoylamino)phenyl]-1H-naphtho[1,2-b][1,4]diazepine-2,4(3H,5H)-dione;
(Compound B72) 5-[2-(2-iodobenzoyl)aminoethyl]-1H-naphtho[1,2-b][1,4]diazepine-2,4(3H,5H)-dione;
(Compound B73) 5-[3-[(2-iodobenzoyl)amino]phenyl]-1H-naphtho[1,2-b][1,4]diazepine-2,4(3H,5H)-dione;
(Compound B74) 6,7-dimethyl-1-[4-(2-iodobenzoyl)aminophenyl]-1H-1,5-benzodiazepine-2,4(3H,5H)-dione;
(Compound B75) 5-[4-[(1-methylpiperidin-4-yl)carbonylamino]phenyl]-1H-naphtho[1,2-b][1,4]diazepine-2,4(3H,5H)-dione hydrochloride;
(Compound B76) 5-[4-[(benzofuran-2-yl)carbonylamino]phenyl]-1H-naphtho[1,2-b][1,4]diazepine-2,4(3H,5H)-dione;
(Compound B77) 5-[4-[(1-methyl-1H-indol-3-yl)carbonylamino]phenyl]-1H-naphtho[1,2-b][1,4]diazepine-2,4(3H,5H)-dione;
(Compound B78) 5-[4-(2-propenylbenzoylamino)phenyl]-1H-naphtho[1,2-b][1,4]diazepine-2,4(3H,5H)-dione;
(Compound B79) 5-[4-(2-propylbenzoylamino)phenyl]-1H-naphtho[1,2-b][1,4]diazepine-2,4(3H,5H)-dione;
(Compound B80) 5-[3-fluoro-4-(2-iodobenzoylamino)phenyl]-1H-naphtho[1,2-b][1,4]diazepine-2,4(3H,5H)-dione;
(Compound B81) 5-[4-(2-hydroxy-3-methylbenzoyl)aminophenyl]-1,2,3,4,8,9,10,11-octahydronaphtho[1,2-b][1,4]diazepine-2,4(3H,5H)-dione;
(Compound B82) 5-[4-[(2-isopropoxybenzoyl)amino]phenyl]-1H-naphtho[1,2-b][1,4]diazepine-2,4(3H,5H)-dione;
(Compound B83) 5-[4-[(3-methylthiophen-2-yl)carbonylamino]phenyl]-1H-naphtho[1,2-b][1,4]diazepine-2,4(3H,5H)-dione;
(Compound B84) 5-[4-(2-phenoxypropionylamino)phenyl]-1H-naphtho[1,2-b][1,4]diazepine-2,4(3H,5H)-dione;
(Compound B85) 5-[4-[2-(4-chloro-2-methylphenoxy)acetylamino]phenyl]-1H-naphtho[1,2-b][1,4]diazepine-2,4(3H,5H)-dione;
(Compound B86) 5-[4-[(4-fluoro-2-trifluoromethyl)benzoyl]aminophenyl]-1H-naphtho[1,2-b][1,4]diazepine-2,4(3H,5H)-dione;
(Compound B87) 5-[4-(4-fluoro-2-methoxybenzoyl)aminophenyl]-1H-naphtho[1,2-b][1,4]diazepine-2,4(3H,5H)-dione;
(Compound B88) 5-[4-(4-fluoro-2-hydroxybenzoyl)aminophenyl]-1H-naphtho[1,2-b][1,4]diazepine-2,4(3H,5H)-dione;
(Compound B89) 5-[3-[(2-iodophenylacetyl)amino]phenyl]-1H-naphtho[1,2-b][1,4]diazepine-2,4(3H,5H)-dione;
(Compound B90) 5-[4-(2-methyl-2-phenoxypropionylamino)phenyl]-1H-naphtho[1,2-b][1,4]diazepine-2,4(3H,5H)-dione;
(Compound B91) 5-[4-(2-tert-butylbenzoylamino)phenyl]-1,3-dihydronaphtho[1,2-e]-1,4-diazepin-2-one;
(Compound B92) 5-[4-[(3-dimethylaminobenzoyl)amino]phenyl]-1H-naphtho[1,2-b][1,4]diazepine-2,4(3H,5H)-dione;
(Compound B93) 5-[4-(4-iodo-2-methoxybenzoylamino)phenyl]-1H-naphtho[1,2-b][1,4]diazepine-2,4(3H,5H)-dione;
(Compound B94) 5-[4-(6-fluoro-2-methoxybenzoylamino)phenyl]-1H-naphtho[1,2-b][1,4]diazepine-2,4(3H,5H)-dione;
(Compound B95) 5-[4-(2-hydroxy-4-iodobenzoylamino)phenyl]-1H-naphtho[1,2-b][1,4]diazepine-2,4(3H,5H)-dione;
(Compound B96) 5-[4-(6-fluoro-2-hydroxybenzoylamino)phenyl]-1H-naphtho[1,2-b][1,4]diazepine-2,4(3H,5H)-dione;
(Compound B97) 5-[4-(2-fluorobenzoyl)aminophenyl]-1H-naphtho[1,2-b][1,4]diazepine-2,4(3H,5H)-dione;
(Compound B98) 5-[4-[(2-dimethylaminobenzoyl)amino]phenyl]-1H-naphtho[1,2-b][1,4]diazepine-2,4(3H,5H)-dione;
(Compound B99) 5-[4-(2-methoxy-6-methylbenzoylamino)phenyl]-1H-naphtho[1,2-b][1,4]diazepine-2,4(3H,5H)-dione;
(Compound B100) 5-[4-(2-hydroxy-6-methylbenzoylamino)phenyl]-1H-naphtho[1,2-b][1,4]diazepine-2,4(3H,5H)-dione;
(Compound B101) 5-[4-[3-(2-methylphenyl)propionylamino]phenyl]-1H-naphtho[1,2-b][1,4]diazepine-2,4(3H,5H)-dione;
(Compound B102) 5-(4-phenylcarbamoylphenyl)-1H-naphtho[1,2-b][1,4]diazepine-2,4(3H,5H)-dione;
(Compound B103) 5-(4-benzylcarbamoylphenyl)-1H-naphtho[1,2-b][1,4]diazepine-2,4(3H,5H)-dione;
(Compound B104) 5-[4-[3-(2-methylphenyl)propenoylamino]phenyl]-1H-naphtho[1,2-b][1,4]diazepine-2,4(3H,5H)-dione;
(Compound B105) 5-[4-[3-(2-chlorophenyl)propionylamino]phenyl]-1H-naphtho[1,2-b][1,4]diazepine-2,4(3H,5H)-dione;
(Compound B106) 5-[4-(2-iodobenzoyl)aminophenyl]-1,2,3,4,8,9,10,11-octahydronaphtho[1,2-b][1,4]diazepine-2,4(3H,5H)-dione;
(Compound B107) 5-[4-[(1-methyl-1H-pyrrol-2-ylacetyl)amino]phenyl]-1,2,3,4,8,9,10,11-octahydronaphtho[1,2-b][1,4]diazepine-2,4(3H,5H)-dione;
(Compound B108) 5-[4-(2-chlorobenzyl)carbamoylphenyl]-1H-naphtho[1,2-b][1,4]diazepine-2,4(3H,5H)-dione;
(Compound B109) 5-[4-[3-(2-chlorophenyl)propenoylamino]phenyl]-1H-naphtho[1,2-b][1,4]diazepine-2,4(3H,5H)-dione;
(Compound B110) 5-[4-(2-chlorophenyl)carbamoylphenyl]-1H-naphtho[1,2-b][1,4]diazepine-2,4(3H,5H)-dione;
(Compound B111) 5-[4-(6-bromo-2,3-methylenedioxybenzoylamino)phenyl]-1H-naphtho[1,2-b][1,4]diazepine-2,4(3H,5H)-dione;
(Compound B112) 5-[4-(6-bromo-2-methoxybenzoylamino)phenyl]-1H-naphtho[1,2-b][1,4]diazepine-2,4(3H,5H)-dione;
(Compound B113) 5-[4-[(2-tert-butylbenzoyl)amino]phenyl]-1,2,3,4,8,9,10,11-octahydronaphtho[1,2-b][1,4]diazepine-2,4(3H,5H)-dione;
(Compound B114) 5-[2-(2-iodobenzoyl)aminopyridin-5-yl]-1H-naphtho[1,2-b][1,4]diazepine-2,4(3H,5H)-dione;
(Compound B115) 5-[4-(6-bromo-2-hydroxybenzoylamino)phenyl]-1H-naphtho[1,2-b][1,4]diazepine-2,4(3H,5H)-dione;
(Compound B116) 5-[4-(6-chloro-2-methoxybenzoylamino)phenyl]-1H-naphtho[1,2-b][1,4]diazepine-2,4(3H,5H)-dione;
(Compound B117) 5-[4-(2-iodobenzoylamino)phenyl]-1H-[1,4]diazepino[2,3-h]quinoline-2,4(3H,5H)-dione;
(Compound B118) 5-[4-(6-chloro-2-hydroxybenzoylamino)phenyl]-1H-naphtho[1,2-b][1,4]diazepine-2,4(3H,5H)-dione;
(Compound B119) 5-[4-(2-hydroxy-6-methoxybenzoylamino)phenyl]-1H-naphtho[1,2-b][1,4]diazepine-2,4(3H,5H)-dione;
(Compound B120) 5-[4-[2-methoxy-6-(trifluoromethyl)benzoylamino]phenyl]-1H-naphtho[1,2-b][1,4]diazepine-2,4(3H,5H)-dione;
(Compound B121) 5-[4-[2-hydroxy-6-(trifluoromethyl)benzoylamino]phenyl]-1H-naphtho[1,2-b][1,4]diazepine-2,4(3H,5H)-dione;
(Compound B122) 5-[4-[(2-isopropenylbenzoyl)amino]phenyl]-1,2,3,4,8,9,10,11-octahydronaphtho[1,2-b][1,4]diazepine-2,4(3H,5H)-dione;
(Compound B123) 5-[4-[(2-isopropylbenzoyl)amino]phenyl]-1,2,3,4,8,9,10,11-octahydronaphtho[1,2-b][1,4]diazepine-2,4(3H,5H)-dione;
(Compound B124) 5-[4-[2-chloro-5-(methylthio)benzoylamino]phenyl]-1H-naphtho[1,2-b][1,4]diazepine-2,4(3H,5H)-dione;
(Compound B125) 5-[4-[2-(methylthio)benzoylamino]phenyl]-1H-naphtho[1,2-b][1,4]diazepine-2,4(3H,5H)-dione;
(Compound B126) 5-[4-[3-(methylthio)benzoylamino]phenyl]-1H-naphtho[1,2-b][1,4]diazepine-2,4(3H,5H)-dione;
(Compound B127) 5-[4-[2-ethyl-6-methoxybenzoylamino]phenyl]-1H-naphtho[1,2-b][1,4]diazepine-2,4(3H,5H)-dione;
(Compound B128) 5-[4-(3-methanesulfonylbenzoylamino)phenyl]-1H-naphtho[1,2-b][1,4]diazepine-2,4(3H,5H)-dione;
(Compound B129) 6-ethyl-1-[4-(2-iodobenzoyl)aminophenyl]-1H-1,5-benzodiazepine-2,4(3H,5H)-dione;
(Compound B130) 5-[4-[2-ethyl-6-hydroxybenzoylamino]phenyl]-1H-naphtho[1,2-b][1,4]diazepine-2,4(3H,5H)-dione;
(Compound B131) 5-[4-(3-methanesulfinylbenzoylamino)phenyl]-1H-naphtho[1,2-b][1,4]diazepine-2,4(3H,5H)-dione;
(Compound B132) 5-[4-(2-chloro-5-methanesulfinylbenzoylamino)phenyl]-1H-naphtho[1,2-b][1,4]diazepine-2,4(3H,5H)-dione;
(Compound B133) 5-[4-(2-methanesulfinylbenzoylamino)phenyl]-1H-naphtho[1,2-b][1,4]diazepine-2,4(3H,5H)-dione;
(Compound B134) 5-[4-[[2-(4-morpholinyl)acetyl]amino]phenyl]-1,2,3,4,8,9,10,11-octahydronaphtho[1,2-b][1,4]diazepine-2,4(3H,5H)-dione hydrochloride;
(Compound B135) 5-[4-(2-chloro-6-methoxybenzoylamino)phenyl]-1,3-dihydronaphtho[1,2-e]-1,4-diazepin-2-one;
(Compound B136) 5-[4-[[(3-chloropyridin-2-yl)carbonyl]amino]phenyl]-1H-naphtho[1,2-b][1,4]diazepine-2,4(3H,5H)-dione;
(Compound B137) 5-[4-(2-chloro-6-hydroxybenzoylamino)phenyl]-1,3-dihydronaphtho[1,2-e]-1,4-diazepin-2-one;
(Compound B138) 5-[4-(3-chloro-2-methoxybenzoylamino)phenyl]-1H-naphtho[1,2-b][1,4]diazepine-2,4(3H,5H)-dione;
(Compound B139) 5-[4-[(3-methylpyridin-2-yl)carbonylamino]phenyl]-1H-naphtho[1,2-b][1,4]diazepine-2,4(3H,5H)-dione;
(Compound B140) 5-[4-[[(3-chloropyridin-2-yl)carbonyl]amino]phenyl]-1,2,3,4,8,9,10,11-octahydronaphtho[1,2-b][1,4]diazepine-2,4(3H,5H)-dione;
(Compound B141) 5-[4-(3-chloro-2-hydroxybenzoylamino)phenyl]-1H-naphtho[1,2-b][1,4]diazepine-2,4(3H,5H)-dione;
(Compound B142) 5-[4-[[(3-hydroxypyridin-2-yl)carbonyl]amino]phenyl]-1H-naphtho[1,2-b][1,4]diazepine-2,4(3H,5H)-dione;
(Compound B143) 5-[4-[(3-vinylpyridin-2-yl)carbonylamino]phenyl]-1H-naphtho[1,2-b][1,4]diazepine-2,4(3H,5H)-dione;
(Compound B144) 5-[4-[(3-ethylpyridin-2-yl)carbonylamino]phenyl]-1H-naphtho[1,2-b][1,4]diazepine-2,4(3H,5H)-dione;
(Compound B145) N-[4-(2,4-dioxo-1,2,3,4-tetrahydronaphtho-[1,2-b][1,4]-diazepin-5-yl)phenyl]-2-nitrobenzenesulfonamide;
(Compound B146) N-[4-(2,4-dioxo-1,2,3,4-tetrahydronaphtho[1,2-b][1,4]diazepin-5-yl)phenyl]benzenesulfonamide;
(Compound B147) 3-bromo-N-[4-(2,4-dioxo-1,2,3,4-tetrahydronaphtho[1,2-b][1,4]diazepin-5-yl)phenyl]benzenesulfonamide;
(Compound B148) N-[4-(2,4-dioxo-1,2,3,4-tetrahydronaphtho[1,2-b][1,4]diazepin-5-yl)phenyl]-3-methoxybenzenesulfonamide;
(Compound B149) N-[3-(2-oxo-2,3-dihydro-1H-naphtho[1,2-e][1,4]diazepin-5-yl)phenyl]benzenesulfonamide;
(Compound B150) N-[3-(2,4-dioxo-1,2,3,4-tetrahydronaphtho-[1,2-b][1,4]-diazepin-5-yl)phenyl]-2-nitrobenzenesulfonamide;
(Compound B151) N-[4-(2,4-dioxo-1,2,3,4,8,9,10,11-octahydro-naphtho[1,2-b][1,4]-diazepin-5-yl)phenyl]-2-nitrobenzenesulfonamide;
(Compound B152) N-[4-(2,4-dioxo-1,2,3,4,8,9,10,11-octahydro-naphtho[1,2-b][1,4]-diazepin-5-yl)phenyl]-N-methyl-2-nitrobenzenesulfonamide;
(Compound B153) N-[3-(2,4-dioxo-1,2,3,4-tetrahydronaphtho-[1,2-b][1,4]-diazepin-5-yl)phenyl]-N-methyl-2-nitrobenzenesulfonamide;
(Compound B154) 4-(2,4-dioxo-1,2,3,4,8,9,10,11-octahydronaphtho[1,2-b][1,4]diazepin-5-yl)-N-phenylbenzenesulfonamide;
(Compound B155) N-[3-(2,4-dioxo-1,2,3,4-tetrahydronaphtho[1,2-b]-[1,4]diazepin-5-yl)phenyl]-2-naphthalenesulfonamide;
(Compound B156) N-[3-(2,4-dioxo-1,2,3,4-tetrahydronaphtho[1,2-b]-[1,4]diazepin-5-yl)phenyl]-1-naphthalenesulfonamide;
(Compound B157) N-[4-(2,4-dioxo-1,2,3,4-tetrahydronaphtho[1,2-b][1,4]diazepin-5-yl)phenyl]cyclohexanesulfonamide;
(Compound B158) N-[4-(2,4-dioxo-1,2,3,4-tetrahydronaphtho[1,2-b][1,4]diazepin-5-yl)phenyl]-3-pyridinesulfonamide hydrochloride;
(Compound B159) N-[4-(2,4-dioxo-1,2,3,4-tetrahydronaphtho[1,2-b][1,4]diazepin-5-yl)phenyl]-4-isopropylbenzenesulfonamide;
(Compound B160) N-[4-(2,4-dioxo-1,2,3,4-tetrahydronaphtho[1,2-b][1,4]diazepin-5-yl)phenyl]phenylmethanesulfonamide;
(Compound B161) N-[4-(2,4-dioxo-1,2,3,4-tetrahydronaphtho[1,2-b][1,4]diazepin-5-yl)phenyl]-2-thiophene-sulfonamide;
(Compound B162) N-[4-(2,4-dioxo-1,2,3,4-tetrahydronaphtho[1,2-b][1,4]diazepin-5-yl)phenyl]-2-naphthalenesulfonamide;
(Compound B163) 4-(2,4-dioxo-1,2,3,4,8,9,10,11-octahydronaphtho-[1,2-b][1,4]diazepin-5-yl)phenyl 3-bromobenzene-sulfonate;
(Compound B164) N-benzyl-N-[4-(1-benzyl-2,4-dioxo-1,2,3,4-tetrahydronaphtho[1,2-b][1,4]diazepin-5-yl)phenyl]-2-nitrobenzenesulfonamide;
(Compound B165) N-benzyl-N-[4-(2,4-dioxo-1,2,3,4-tetrahydronaphtho[1,2-b][1,4]diazepin-5-yl)phenyl]-2-nitrobenzenesulfonamide;
(Compound B166) 3-bromo-N-[4-(2,4-dioxo-1,2,3,4-tetrahydronaphtho[1,2-b][1,4]diazepin-5-yl)phenyl]-N-methylbenzenesulfonamide;
(Compound B167) N-[4-(2,4-dioxo-1,2,3,4-tetrahydronaphtho-[1,2-b][1,4]-diazepin-5-yl)phenyl]-N-methyl-2-nitrobenzenesulfonamide;
(Compound B168) N-[4-(2,4-dioxo-1,2,3,4-tetrahydronaphtho-[1,2-b][1,4]-diazepin-5-yl)phenyl]-N-(2-hydroxyethyl)-2-nitrobenzenesulfonamide;
(Compound B169) N-[4-(7-chloro-2,4-dioxo-2,3,4,5-tetrahydro-1H-benzo[b][1,4]diazepin-1-yl)phenyl]benzenesulfonamide;
(Compound B170) N-[4-(7-bromo-2,4-dioxo-2,3,4,5-tetrahydro-1H-benzo[b][1,4]diazepin-1-yl)phenyl]benzenesulfonamide;
(Compound B171) N-[4-[(2,4-dioxo-7-(trifluoromethyl)-2,3,4,5-tetrahydro-1H-benzo[b][1,4]diazepin-1-yl)]phenyl]benzenesulfonamide;
(Compound B172) N-[4-(2,4-dioxo-2,3,4,5-tetrahydro-1H-benzo[b][1,4]diazepin-1-yl)phenyl]benzenesulfonamide;
(Compound B173) 1-(2-chlorophenyl)-N-[4-(2,4-dioxo-1,2,3,4-tetrahydronaphtho[1,2-b][1,4]diazepin-5-yl)phenyl]methanesulfonamide;
(Compound B174) 1-(3-bromophenyl)-N-[4-(2,4-dioxo-1,2,3,4-tetrahydronaphtho[1,2-b][1,4]diazepin-5-yl)phenyl]methanesulfonamide;
(Compound B175) N-[4-(2,4-dioxo-1,2,3,4-tetrahydronaphtho-[1,2-b][1,4]-diazepin-5-yl)phenyl]-2-trifluoromethylbenzenesulfonamide;
(Compound B176) N-[4-(7-bromo-6-methyl-2,4-dioxo-2,3,4,5-tetrahydro-1H-benzo[b][1,4]diazepin-1-yl)phenyl]benzenesulfonamide;
(Compound B177) 1-(2-chlorophenyl)-N-[4-(2,4-dioxo-1,2,3,4,8,9,10,11-octahydronaphtho[1,2-b][1,4]diazepin-5-yl)phenyl]methanesulfonamide;
(Compound B178) 3-bromo-N-[4-(2,4-dioxo-1,2,3,4,8,9,10,11-octahydronaphtho[1,2-b][1,4]diazepin-5-yl)phenyl]benzenesulfonamide;
(Compound B179) N-[4-(2,4-dioxo-1,2,3,4,8,9,10,11-octahydronaphtho[1,2-b][1,4]diazepin-5-yl)phenyl]-3-methoxybenzenesulfonamide;
(Compound B180) 1-(2-bromophenyl)-N-[4-(2,4-dioxo-1,2,3,4-tetrahydronaphtho[1,2-b][1,4]diazepin-5-yl)phenyl]methanesulfonamide;
(Compound B181) N-[4-(2,4-dioxo-1,2,3,4-tetrahydronaphtho[1,2-b][1,4]diazepin-5-yl)phenyl]-1-(2-methylphenyl)methanesulfonamide;
(Compound B182) N-[4-(2,4-dioxo-1,2,3,4-tetrahydronaphtho[1,2-b][1,4]diazepin-5-yl)phenyl]-1-(2-nitrophenyl)methanesulfonamide;
(Compound B183) N-[4-(2,4-dioxo-1,2,3,4-tetrahydronaphtho[1,2-b][1,4]diazepin-5-yl)phenyl]-2-phenylethanesulfonamide;
(Compound B184) 1-(2,3-dichlorophenyl)-N-[4-(2,4-dioxo-1,2,3,4,8,9,10,11-octahydronaphtho[1,2-b][1,4]diazepin-5-yl)phenyl]methanesulfonamide;
(Compound B185) 1-(2-chlorophenyl)-N-[4-(2,4-dioxo-7-methoxy-1H-benzo[1,2-b][1,4]diazepin-1-yl)phenyl]methanesulfonamide;
(Compound B186) 1-(2-chlorophenyl)-N-[4-(2,4-dioxo-7-hydroxy-1H-benzo[1,2-b][1,4]diazepin-1-yl)phenyl]methanesulfonamide;
(Compound B187) 1-(4-chlorophenyl)-N-[4-(2,4-dioxo-1,2,3,4-tetrahydronaphtho[1,2-b][1,4]diazepin-5-yl)phenyl]methanesulfonamide;
(Compound B188) 1-(2-chlorophenyl)-N-[4-(2,4-dioxo-1,2,3,4-tetrahydronaphtho[1,2-b][1,4]diazepin-5-yl)benzyl]methanesulfonamide;
(Compound B189) 1-(2-chlorophenyl)-N-[4-(2,4-dioxo-1,2,3,4-tetrahydronaphtho[1,2-b][1,4]diazepin-5-yl)-2-methoxyphenyl]methanesulfonamide;
(Compound B190) 1-(2-chlorophenyl)-N-[4-(2,4-dioxo-1,2,3,4-tetrahydronaphtho[1,2-b][1,4]diazepin-5-yl)-2-hydroxyphenyl]methanesulfonamide;
(Compound B191) 1-(2,6-dichlorophenyl)-N-[4-(2,4-dioxo-1,2,3,4,8,9,10,11-octahydronaphtho[1,2-b][1,4]diazepin-5-yl)phenyl]methanesulfonamide;
(Compound B192) 1-(2-chlorophenyl)-N-[4-(2,4-dioxo-6-methyl-1H-benzo[1,2-b][1,4]diazepin-1-yl)phenyl]methanesulfonamide;
(Compound B193) 1-(2-chlorophenyl)-N-[3-(2,4-dioxy-1,2,3,4-tetrahydronaphtho[1,2-b][1,4]diazepin-5-yl) propyl]methanesulfonamide;
(Compound B194) 1-(2-chlorophenyl)-N-[2-(2,4-dioxo-1,2,3,4-tetrahydronaphtho[1,2-b][1,4]diazepin-5-yl)ethyl]methanesulfonamide;
(Compound B195) N-[4-(2,4-dioxo-1,2,3,4,8,9,10,11-octahydronaphtho[1,2-b][1,4]diazepin-5-yl)phenyl]-1-(2-iodophenyl)methanesulfonamide;
(Compound B196) 1-(2-chlorophenyl)-N-[4-(2,4-dioxo-1,2,3,4,8,9,10,11-octahydronaphtho[1,2-b][1,4]diazepin-5-yl)phenyl]-N-methylmethanesulfonamide;
(Compound B197) 1-(2-chlorophenyl)-N-[4-(2-oxo-2,3-dihydro-1H-naphtho[1,2-e][1,4]diazepin-5-yl)phenyl]methanesulfonamide;
(Compound B198) 1-[2-(trifluoromethyl)phenyl]-N-[4-(2,4-dioxo-1,2,3,4-tetrahydronaphtho[1,2-b][1,4]diazepin-5-yl)phenyl]phenyl-N-methylmethanesulfonamide;
(Compound B199) 1-(2-ethylphenyl)-N-[4-(2,4-dioxo-1,2,3,4-tetrahydronaphtho[1,2-b][1,4]diazepin-5-yl)phenyl]phenyl-N-methylmethanesulfonamide;
(Compound B200) 1-(2,3-dimethylphenyl)-N-[4-(2,4-dioxo-1,2,3,4-tetrahydronaphtho[1,2-b][1,4]diazepin-5-yl)phenyl]phenyl-N-methylmethanesulfonamide;
(Compound B201) 2-(2-chlorophenyl)-N-[4-(2,4-dioxo-1,2,3,4-tetrahydronaphtho[1,2-b][1,4]diazepin-5-yl)phenyl]phenyl-N-methylethanesulfonamide;
(Compound B202) 1-(2-nitrophenyl)-N-[4-(2,4-dioxo-1,2,3,4-tetrahydronaphtho[1,2-b][1,4]diazepin-5-yl)phenyl]phenyl-N-methylmethanesulfonamide;
(Compound B203) 1-(2-aminophenyl)-N-[4-(2,4-dioxo-1,2,3,4-tetrahydronaphtho[1,2-b][1,4]diazepin-5-yl)phenyl]phenyl-N-methylmethanesulfonamide;
(Compound B204) 1-(2-dimethylaminophenyl)-N-[4-(2,4-dioxo-1,2,3,4-tetrahydronaphtho[1,2-b][1,4]diazepin-5-yl)phenyl]phenyl-N-methylmethanesulfonamide;
(Compound B205) 5-[4-[(pyridin-4-yl)carbonylamino]phenyl]-1H-naphtho[1,2-b][1,4]diazepine-2,4(3H,5H)-dione hydrochloride;
(Compound B206) 5-[4-[2-[(pyridin-3-yl)oxy]acetylamino]phenyl]-1H-naphtho[1,2-b][1,4]diazepine-2,4(3H,5H)-dione hydrochloride;
(Compound B207) 5-[4-[(pyridin-3-yl)carbonylamino]phenyl]-1H-naphtho[1,2-b][1,4]diazepine-2,4(3H,5H)-dione hydrochloride;
(Compound B208) 5-[4-[(2-methylpyridin-3-yl)carbonylamino]phenyl]-1H-naphtho[1,2-b][1,4]diazepine-2,4(3H,5H)-dione hydrochloride;
(Compound B209) 5-[4-[(2-chloropyridin-3-yl)carbonylamino]phenyl]-1H-naphtho[1,2-b][1,4]diazepine-2,4(3H,5H)-dione;
(Compound B210) 5-[4-[2-[(pyridin-2-yl)oxy]acetylamino]phenyl]-1H-naphtho[1,2-b][1,4]diazepine-2,4(3H,5H)-dione;
(Compound B211) 5-[4-[[4-(trifluoromethyl)pyridin-3-yl]carbonylamino]phenyl]-1H-naphtho[1,2-b][1,4]diazepine-2,4(3H,5H)-dione;
(Compound B212) 5-[4-[(2-chloropyridin-3-yl)carbonylamino]phenyl]-1H-[1,4]diazepino[2,3-f]isoquinoline-2,4(3H,5H)-dione;
(Compound B213) 5-[4-[(2-chloropyridin-3-yl)carbonylamino]phenyl]-8,9,10,11-tetrahydro-1H-[1,4]diazepino[2,3-f]isoquinoline-2,4(3H,5H)-dione; and
(Compound B214) 5-[4-[(2-isopropylbenzoyl)amino]phenyl]-1H-naphtho[1,2-b][1,4]diazepine-2,4(3H,5H)-dione

A more preferable compound or a pharmaceutically acceptable salt thereof that can be used as the active ingredient of the medicament of the present invention is included in the compounds represented by the general formulas (A) to (BII) or a pharmaceutically acceptable salt thereof. Specific examples of the more preferable compound or a pharmaceutically acceptable salt thereof can include 5-[3-(1H-tetrazol-5-yl)phenyl]-1H-naphtho[1,2-b][1,4]diazepine-2,4(3H,5H)-dione; a 5-[3-(1H-tetrazol-5-yl)phenyl]-1H-naphtho[1,2-b][1,4]diazepine-2,4(3H,5H)-dione sodium salt; a 5-[3-(1H-tetrazol-5-yl)phenyl]-1H-naphtho[1,2-b][1,4]diazepine-2,4(3H,5H)-dione potassium salt; 5-[3-(5-thioxo-4H-[1,2,4]oxadiazol-3-yl)phenyl]-1H-naphtho[1,2-b][1,4]diazepine-2,4(3H,5H)-dione; a 5-[3-(5-thioxo-4H-[1,2,4]oxadiazol-3-yl)phenyl]-1H-naphtho[1,2-b][1,4]diazepine-2,4(3H,5H)-dione sodium salt; 5-[4-fluoro-3-(1H-tetrazol-5-yl)phenyl]-1H-naphtho[1,2-b][1,4]diazepine-2,4(3H,5H)-dione; a 5-[4-fluoro-3-(1H-tetrazol-5-yl)phenyl]-1H-naphtho[1,2-b][1,4]diazepine-2,4(3H,5H)-dione sodium salt; 5-[4-[2-(trifluoromethyl)benzoyl]aminophenyl]-1H-naphtho[1,2-b][1,4]diazepine-2,4(3H,5H)-dione; 5-[4-[(2-chlorophenylacetyl)amino]phenyl]-1H-naphtho[1,2-b][1,4]diazepine-2,4(3H,5H)-dione; 1-[4-(2,4-dioxo-1,2,3,4-tetrahydronaphtho[1,2-b][1,4]diazepin-5-yl)phenyl]-3-phenylurea; 5-[4-(2-iodobenzoylamino)phenyl]-1H-naphtho[1,2-b][1,4]diazepine-2,4(3H,5H)-dione; 5-[4-[(2-ethylbenzoyl)amino]phenyl]-1H-naphtho[1,2-b][1,4]diazepine-2,4(3H,5H)-dione; 5-[4-(2-tert-butylbenzoylamino)phenyl]-1H-naphtho[1,2-b][1,4]diazepine-2,4(3H,5H)-dione; 5-[4-(2-iodobenzoyl)aminophenyl]-1,2,3,4,8,9,10,11-octahydronaphtho[1,2-b][1,4]diazepine-2,4(3H,5H)-dione; 5-[4-(6-chloro-2-hydroxybenzoylamino)phenyl]-1H-naphtho[1,2-b][1,4]diazepine-2,4(3H,5H)-dione; N-[4-(2,4-dioxo-1,2,3,4-tetrahydronaphtho[1,2-b][1,4]diazepin-5-yl)phenyl]benzenesulfonamide; 1-(2-chlorophenyl)-N-[4-(2,4-dioxo-1,2,3,4-tetrahydronaphtho[1,2-b][1,4]diazepin-5-yl)phenyl]methanesulfonamide; 1-(2-chlorophenyl)-N-[4-(2,4-dioxo-1,2,3,4,8,9,10,11-octahydronaphtho[1,2-b][1,4]diazepin-5-yl)phenyl]methanesulfonamide; 1-(2-bromophenyl)-N-[4-(2,4-dioxo-1,2,3,4-tetrahydronaphtho[1,2-b][1,4]diazepin-5-yl)phenyl]methanesulfonamide; N-[4-(2,4-dioxo-1,2,3,4-tetrahydronaphtho[1,2-b][1,4]diazepin-5-yl)phenyl]-1-(2-methylphenyl)methanesulfonamide; N-[4-(2,4-dioxo-1,2,3,4-tetrahydronaphtho[1,2-b][1,4]diazepin-5-yl)phenyl]-2-phenylethanesulfonamide; 1-(2-chlorophenyl)-N-[4-(2,4-dioxo-1,2,3,4,8,9,10,11-octahydronaphtho[1,2-b][1,4]diazepin-5-yl)phenyl]-N-methylmethanesulfonamide; 1-(2-chlorophenyl)-N-[4-(2-oxo-2,3-dihydro-1H-naphtho[1,2-e][1,4]diazepin-5-yl)phenyl]methanesulfonamide; 5-[4-[(2-methylpyridin-3-yl)carbonylamino]phenyl]-1H-naphtho[1,2-b][1,4]diazepine-2,4(3H,5H)-dione hydrochloride; 5-[4-[(2-chloropyridin-3-yl)carbonylamino]phenyl]-1H-naphtho[1,2-b][1,4]diazepine-2,4(3H,5H)-dione; 5-[4-[2-[(pyridin-2-yl)oxy]acetylamino]phenyl]-1H-naphtho[1,2-b][1,4]diazepine-2,4(3H,5H)-dione; and 5-[4-[(2-isopropylbenzoyl)amino]phenyl]-1H-naphtho[1,2-b][1,4]diazepine-2,4(3H,5H)-dione. Note that the active ingredient of the medicament of the present invention is not limited to the above specific compounds or a pharmaceutically acceptable salt thereof.

An even more preferable compound or a pharmaceutically acceptable salt thereof that can be used as the active ingredient of the medicament of the present invention is included in the compounds represented by the general formulas (A) to (BII) or a pharmaceutically acceptable salt thereof. Specific examples of the even more preferable compound or a pharmaceutically acceptable salt thereof can include 5-[3-(1H-tetrazol-5-yl)phenyl]-1H-naphtho[1,2-b][1,4]diazepine-2,4(3H,5H)-dione; a 5-[3-(1H-tetrazol-5-yl)phenyl]-1H-naphtho[1,2-b][1,4]diazepine-2,4(3H,5H)-dione sodium salt; 5-[3-(5-thioxo-4H-[1,2,4]oxadiazol-3-yl)phenyl]-1H-naphtho[1,2-b][1,4]diazepine-2,4(3H,5H)-dione; a 5-[3-(5-thioxo-4H-[1,2,4]oxadiazol-3-yl)phenyl]-1H-naphtho[1,2-b][1,4]diazepine-2,4(3H,5H)-dione sodium salt; 5-[4-[2-(trifluoromethyl)benzoyl]aminophenyl]-1H-naphtho[1,2-b][1,4]diazepine-2,4(3H,5H)-dione; 5-[4-(2-iodobenzoylamino)phenyl]-1H-naphtho[1,2-b][1,4]diazepine-2,4(3H,5H)-dione; and 5-[4-(6-chloro-2-hydroxybenzoylamino)phenyl]-1H-naphtho[1,2-b][1,4]diazepine-2,4(3H,5H)-dione. Note that the active ingredient of the medicament of the present invention is not limited to the above specific compounds or a pharmaceutically acceptable salt thereof.

Stereoisomers of the compounds represented by the general formulas (A) to (BII), such as a cis-trans isomer, an optically active form, or a racemic form thereof, may exist as well, all of which are included in the present invention.

Tautomers of the compounds represented by the general formulas (A) to (BII) may exist as well, and the tautomers show the same activities as those of the compounds and are included in the present invention.

Furthermore, the compounds represented by the general formulas (A) to (BII) may have one or two or more asymmetric carbons depending on the type of substituent, and any optical isomer or any mixture or a racemic form of optical isomers, which is based on the asymmetric carbon, or a diastereoisomer or any mixture of diastereoisomers, which is based on two or more asymmetric carbons, may also be used as the active ingredient of the medicament of the present invention. In a case where the compounds represented by the general formulas (AI) to (BII) have a double bond or a cyclic structure, a geometric isomer may exist, and, in addition to a pure form of the geometric isomer, a mixture of geometric isomers, which are contained in the mixture at any ratio, may also be used as the active ingredient of the medicament of the present invention.

In addition to the compounds represented by the general formulas (A) to (BII), a free form of the compound or any solvate of a salt form of the compound may also be used as active ingredient of the medicament of the present invention. The solvate also includes a hydrate.

In the present specification, the compounds represented by the general formulas (A) to (BII) are not particularly limited, and the compounds represented by the general formulas (A) to (BII) can include the stereoisomer of the compound, such as the cis-trans isomer, the optically active form, or the racemic form thereof; the tautomer of the compound; any optical isomer or racemic form of the compound, which is based on an asymmetric carbon; and the diastereoisomer of the compound and a mixture thereof, which is based on two or more asymmetric carbons.

In addition to the compounds represented by the general formulas (A) to (BII), acid addition salts or base addition salts of these compounds may also be used as the active ingredient of the medicament of the present invention. As the acid addition salt, for example, a mineral acid salt such as hydrochloride, sulfate, and nitrate; an organic acid salt such as methanesulfonate, p-toluenesulfonate, oxalate, and malate; and the like can be used, however, the acid addition salt is not limited thereto. Examples of the base addition salt can include a metal salt such as a lithium salt, a sodium salt, a potassium salt, a magnesium salt, and a calcium salt; an ammonium salt; an organic amine salt such as a triethylamine salt and an ethanolamine salt; and the like, however, the base addition salt is not limited thereto. Among these salts, a pharmaceutically acceptable salt is preferably used as the active ingredient of the medicament of the present invention.

The medicament provided by the present invention can be used in preventing or treating nociceptive pain, particularly, inflammatory pain. Furthermore, the medicament provided by the present invention can be used for preventing or treating the inflammatory pain, in which the inflammatory pain is pain in inflammatory bowel disease. Furthermore, the medicament provided by the present invention can be used for, for example, preventing or treating the inflammatory pain in inflammatory bowel disease, in which the inflammatory pain in inflammatory bowel disease is inflammatory pain in Crohn's disease, ulcerative colitis, simple ulcer, or non-specific multiple ulcers of the small intestine.

Alternatively/Furthermore, the medicament provided by the present invention can be used in preventing or treating visceral pain, particularly, visceral pain caused by the intestinal tract. Furthermore, the medicament provided by the present invention can be used for preventing or treating the visceral pain caused by the intestinal tract, in which the visceral pain caused by the intestinal tract is visceral pain caused by inflammatory bowel disease. Furthermore, the medicament provided by the present invention can be used for, for example, preventing or treating the visceral pain caused by inflammatory bowel disease, in which the visceral pain caused by inflammatory bowel disease is visceral pain caused by Crohn's disease, ulcerative colitis, simple ulcer, or non-specific multiple ulcers of the small intestine.

Furthermore, the medicament provided by the present invention can be used for preventing or treating the visceral pain, in which the visceral pain is pain after remission or curing of inflammation.

The medicament provided by the present invention shows significant analgesic action compared to a DNBS+vehicle group in an acute administration test. In addition, the medicament provided by the present invention was shown to have a higher analgesic effect than a DNBS+dexamathasone group.

Thus, it is suggested that, in one aspect, the medicament provided by the present invention has an analgesic action on inflammatory pain and visceral pain in addition to the action of suppressing inflammation.

The medicament provided by the present invention shows significant analgesic action compared to a DNBS+vehicle group in a preventive administration test. In addition, the medicament provided by the present invention was shown to have a similar degree of analgesic effect as compared to a DNBS+dexamathasone group.

Thus, it is suggested that the medicament provided by the present invention has an analgesic action on inflammatory pain and/or visceral pain while suppressing inflammation. Furthermore, it is suggested that, in one aspect, the medicament provided by the present invention is used in treatment by remission maintenance therapy.

The medicament provided by the present invention shows significant analgesic action compared to a DNBS+vehicle group in an intervention test. In addition, the medicament provided by the present invention was shown to have a higher analgesic effect than a DNBS+dexamathasone group.

Thus, it is suggested that, in one aspect, the medicament provided by the present invention has an effect of repairing a tissue damage after colitis (including inflammatory bowel disease) is already established. The assertion of this effect can also be supported by the fact that the medicament provided by the present invention has an effect of counteracting a decrease in occludin. In addition, it is suggested that, in one aspect, the medicament provided by the present invention has an effect of treating visceral pain after remission or curing of inflammation as well. Furthermore, it is suggested that, in one aspect, the medicament provided by the present invention is used in treatment by remission induction therapy.

As another aspect, the medicament of the present invention can be used for any one or a combination of two or more of the following uses.
Prevention or treatment of irritable bowel syndrome or inflammatory bowel disease
Prevention or treatment of ulcerative colitis or Crohn's disease of the inflammatory bowel disease
Remission induction therapy or remission maintenance therapy in the inflammatory bowel disease
Suppression or reduction of an adhesion or ulcer in an intestinal tissue of irritable bowel syndrome or inflammatory bowel disease
Suppression or reduction of a region of transmural inflammation in an intestinal tissue of irritable bowel syndrome or inflammatory bowel disease
Suppression or reduction of histological damage in an intestinal tissue of the irritable bowel syndrome or the inflammatory bowel disease
Suppression or reduction of mucosal necrosis in an intestinal tissue of irritable bowel syndrome or the inflammatory bowel disease
Suppression or reduction of cellular infiltration in an intestinal tissue of irritable bowel syndrome or inflammatory bowel disease
Protection or maintenance of mucosa in an intestinal tissue of irritable bowel syndrome or the inflammatory bowel disease
Prevention or treatment of the irritable bowel syndrome or the inflammatory bowel disease accompanied by an adhesion or ulcer in an intestinal tissue
Prevention or treatment of the irritable bowel syndrome or the inflammatory bowel disease accompanied by development of a region of transmural inflammation in an intestinal tissue
Prevention or treatment of the irritable bowel syndrome or the inflammatory bowel disease accompanied by histological damage in an intestinal tissue
Prevention or treatment of the irritable bowel syndrome or the inflammatory bowel disease accompanied by necrosis or damage of mucosa in an intestinal tissue
Prevention or treatment of the irritable bowel syndrome or the inflammatory bowel disease accompanied by cellular infiltration in an intestinal tissue
Prevention or treatment of the irritable bowel syndrome or the inflammatory bowel disease accompanied by increase in a proinflammatory cytokine
Prevention or treatment of the irritable bowel syndrome or the inflammatory bowel disease accompanied by increase in interleukin-1β
Prevention or treatment of the irritable bowel syndrome or the inflammatory bowel disease accompanied by colonic damage and increase in a proinflammatory cytokine
Prevention or treatment of the irritable bowel syndrome or the inflammatory bowel disease accompanied by colonic damage and increase in interleukin-1β

Note that the subject of the application of the medicament of the present invention is not limited thereto.

The medicament of the present invention can be administered by oral administration or parenteral administration. The medicament of the present invention can be produced in a suitable dosage form such as a tablet, granules, a powder, a capsule, a suspension, an inhalation, a dry powder inhaler, an inhalation liquid, an aerosol inhaler, an injection, and a suppository, according to a general method in the technical field of preparations.

These preparations can be produced using a general technique. For example, in a case of the tablet, a general pharmaceutically acceptable additive such as a diluent, a disintegrant, a binder, a lubricant, and a coloring agent can be used. Examples of the diluent can include lactose, D-mannitol, microcrystalline cellulose, glucose, and the like. Examples of the disintegrant can include starch, carboxymethylcellulose calcium (CMC-Ca), and the like. Examples of the lubricant can include magnesium stearate, talc, and the like. Examples of the binder can include hydroxypropyl cellulose (HPC), gelatin, polyvinyl pyrrolidone (PVP), and the like.

A pharmaceutically acceptable additive such as a solvent, a stabilizer, a solubilizing agent, a suspension, an emulsifier, an analgesic agent, a buffer, and a preservative is used in preparation of the injection. The pharmaceutically acceptable additive and a method of preparing a preparation can be suitably selected by those skilled in the art.

Types of the inhalation for the parenteral administration include an aerosol, a dry powder for inhalation, a liquid for inhalation (for example, a solution for inhalation, a suspension for inhalation, and the like), and a capsule inhalation, and the liquid for inhalation may be in a form that is used by being dissolved or suspended in water or another suitable medium at the time of use. The inhalation can be applied using a suitable inhalation container. For example, when administering the liquid for inhalation, a sprayer (an atomizer or a nebulizer) or the like can be used, and when administering the dry powder for inhalation, an inhaler for a dry powder drug or the like can be used.

The inhalation can be produced according to a known method. For example, the inhalation is produced by obtaining a dry powder or a liquid of the compounds represented by the general formulas (A) to (BII), blending the dry powder or the liquid of the compounds into an inhalation propellant or a carrier, and filling a suitable inhalation container with the blended product. In a case of powderizing the compounds represented by the general formulas (A) to (BII), the powderization is performed according to a general method. For example, a dry powder is prepared by obtaining a fine powder of the compounds and lactose, starch, magnesium stearate, or the like, thereby obtaining a homogeneous mixture, or by granulating the compounds. In addition, in a case where the compounds represented by the general formulas (A) to (BII) are liquefied, for example, the compounds may be dissolved in a liquid carrier such as water, physiological saline, and an organic solvent. As the propellant, a conventionally known propellant, for example, alternative chlorofluorocarbon, a liquefied gas propellant (for example, fluorohydrocarbon, liquefied petroleum, diethyl ether, dimethyl ether, and the like), a compressed gas (for example, a soluble gas (for example, carbon dioxide gas, nitrous oxide gas, and the like), an insoluble gas (for example, nitrogen gas and the like)), and the like are used.

A pharmaceutically acceptable additive may be suitably blended into the inhalation as necessary. The additive may be any additive that is generally used. For example, a solid diluent (for example, sucrose, lactose, glucose, mannitol, sorbitol, maltose, cellulose, and the like), a liquid diluent (for example, propylene glycol and the like), a binder (starch, dextrin, methylcellulose, hydroxypropylcellulose, polyvinyl pyrrolidone, polyethylene glycol, sucrose, and the like), a lubricant (for example, magnesium stearate, light anhydrous silicic acid, talc, sodium lauryl sulfate, and the like), a corrigent (for example, citric acid, menthol, glycyrrhizin ammonium salt, glycine, an orange powder, and the like), a preservative (for example, sodium benzoate, sodium bisulfite, methylparaben, propylparaben, and the like), a stabilizer (for example, citric acid, sodium citrate, and the like), a suspending agent or an emulsifier (for example, methylcellulose, polyvinyl pyrrolidone, polyvinyl alcohol, lecithin, sorbitan trioleate, and the like), a dispersing agent (for example, a surfactant and the like), a solvent (for example, water and the like), a tonicity agent (for example, sodium chloride, concentrated glycerin, and the like), a pH regulator (for example, hydrochloric acid, sulfuric acid, and the like) a solubilizer (for example, ethanol and the like), an antiseptic agent (benzalkonium chloride, paraben, and the like), a colorant, a buffer agent (sodium phosphate, sodium acetate, and the like), a thickening agent (carboxy vinyl polymer and the like), an absorption promoter, and the like are used. The liquid for inhalation is prepared by, for example, suitably selecting the antiseptic agent, the colorant, the buffer agent, the tonicity agent, the thickening agent, the absorption promoter, or the like as necessary. Furthermore, the dry powder for inhalation is prepared by, for example, suitably selecting the lubricant, the binder, the diluent, the colorant, the antiseptic agent, the absorption promoter (bile salt, chitosan, and the like), or the like as necessary.

Furthermore, in order to prepare the compounds represented by the general formulas (A) to (BII) in a sustained release form, the inhalation may contain a biodegradable polymer. Examples of the biodegradable polymer can include a fatty acid ester polymer or a copolymer thereof, polyacrylic acid esters, polyhydroxybutyric acids, polyalkylene oxalates, polyorthoester, polycarbonate, and polyamino acids, and one polymer can be used alone, or two or more thereof can be used in combination. Furthermore, a phospholipid such as egg yolk lecithin, chitosan, or the like may also be used. Examples of the fatty acid ester polymer or a copolymer thereof can include polylactic acid, polyglycolic acid, polycitric acid, polymalic acid, and a lactic acid-glycolic acid copolymer, and one polymer or copolymer can be used alone, or two or more thereof can be used in combination. In addition, one of poly-α-cyanoacrylic acid ester, poly-β-hydroxybutyric acid, polytrimethylene oxide, polyorthoester, polyorthocarbonate, polyethylenecarbonate, poly-γ-benzyl-L-glutamic acid, and poly-L-alanine can be used alone, or two or more thereof can be used in combination. The fatty acid ester polymer or a copolymer thereof is preferably polylactic acid, polyglycolic acid, or a lactic acid-glycolic acid copolymer, and is more preferably a lactic acid-glycolic acid copolymer. Furthermore, a microsphere or a nanosphere may be prepared by encapsulating a drug using the biodegradable polymer such as a lactic acid-glycolic acid copolymer.

In other words, the medicament of the present invention can be provided as a medicament composition including the active ingredient and the pharmaceutically acceptable additive.

A dose of the medicament of the present invention is not particularly limited, and generally, to an adult,
approximately 0.01 μg to 100 mg, and preferably 0.3 μg to 10 mg can be administered per day as an active ingredient amount, in a case of administration by an inhalation, a dry powder inhaler, an inhalation liquid, and an aerosol inhaler,
approximately 0.01 mg to 100 mg can be administered per day as an active ingredient amount, in a case of administration by an injection, and
approximately 0.01 mg to 2,000 mg can be administered per day, in a case of oral administration. Note that the dose is not limited to the above doses, and the dose can be increased or decreased depending on the age or a symptom.

The medicament of the present invention can be used in combination with another drug that is useful in treating or preventing various types of nociceptive pain, particularly, inflammatory pain. Individual components in such combination can be administered in divided preparations or in a single preparation separately at different times or at the same time, during the period of treatment or prevention. Therefore, it should be understood that the present invention includes both administration at the same time or administrations at different times, and the administration in the present invention should be understood in such a way. A scope of the combination of the medicament of the present invention and another drug that is useful in treating or preventing the various types of nociceptive pain, particularly, inflammatory pain, includes, in principle, a combination with any medicament preparation that is useful in treating or preventing the various types of nociceptive pain, particularly, inflammatory pain. Another drug that is useful in treating or preventing the various types of nociceptive pain, particularly, inflammatory pain, can also include a medicament that is useful in treating or preventing inflammation itself which causes the pain.

Each preparation in the combined preparation according to the present invention can be selected in various forms and can be produced with the above preparations at the same time. Furthermore, a drug combination which includes the medicament of the present invention and another drug for treating or preventing various types of nociceptive pain, particularly, inflammatory pain, can also be easily produced by those skilled in the art according to a conventional method or a conventional art.

The combination includes not only a combination of the medicament of the present invention and one other active substance, but also a combination of the medicament of the present invention and two or more other active substances. There are many examples of the combination of the medicament of the present invention and one or two or more activators selected from the drugs for treating or preventing various types of nociceptive pain, particularly, inflammatory pain.

The present invention has the following aspects.

<1a> A method of preventing or treating nociceptive pain, the method including administering a compound having a P2X4 receptor antagonistic action (for example, the compounds represented by the general formulas (A) to (BII)) or a pharmaceutically acceptable salt thereof to a subject in need of the method (for example, mammals including human) at a dose effective for treating nociceptive pain;

<2a> A method of preventing or treating visceral pain, the method including administering a compound having a P2X4 receptor antagonistic action (for example, the compounds represented by the general formulas (A) to (BII)) or a pharmaceutically acceptable salt thereof to a subject in need of the method (for example, mammals including human) at a dose effective for treating visceral pain;

<3a> The method according to <1a> and <2a> which is a method of preventing or treating the nociceptive pain and the visceral pain;

<4a> The method according to <1a> or <3a>, in which the nociceptive pain is inflammatory pain;

<5a> The method according to <4a>, in which the inflammatory pain is pain in inflammatory bowel disease;

<6a> The method according to <2a> or <3a>, in which the visceral pain is visceral pain caused by the intestinal tract;

<7a> The method according to <6a>, in which the visceral pain caused by the intestinal tract is visceral pain caused by inflammatory bowel disease; and

<8a> The method according to any one of <2a>, <3a>, <6a>, and <7a>, in which the visceral pain is pain after remission or curing of inflammation.

The present invention has the following other aspects.

<1b> A compound having a P2X4 receptor antagonistic action (for example, the compounds represented by the general formulas (A) to (BII)) or a pharmaceutically acceptable salt thereof, which is for use in prevention or treatment of nociceptive pain;

<2b> A compound having a P2X4 receptor antagonistic action (for example, the compounds represented by the general formulas (A) to (BII)) or a pharmaceutically acceptable salt thereof, which is for use in prevention or treatment of visceral pain;

<3b> The compound or a pharmaceutically acceptable salt thereof for the use according to <1b> and <2b>, which is the nociceptive pain and the visceral pain;

<4b> The compound or a pharmaceutically acceptable salt thereof for the use according to <1b> or <3b>, in which the nociceptive pain is inflammatory pain;

<5b> The compound or a pharmaceutically acceptable salt thereof for the use according to <4b>, in which the inflammatory pain is pain in inflammatory bowel disease;

<6b> The compound or a pharmaceutically acceptable salt thereof for the use according to <2b> or <3b>, in which the visceral pain is visceral pain caused by the intestinal tract;

<7b> The compound or a pharmaceutically acceptable salt thereof for the use according to <6b>, in which the visceral pain caused by the intestinal tract is visceral pain caused by inflammatory bowel disease; and

<8b> The compound or a pharmaceutically acceptable salt thereof for the use according to any one of <2b>, <3b>, <6b>, and <7b>, in which the visceral pain is pain after remission or curing of inflammation.

The present invention further has the following other aspects.

<1c> Use of a compound having a P2X4 receptor antagonistic action (for example, the compounds represented by the general formulas (A) to (BII)) or a pharmaceutically acceptable salt thereof for producing a medicament for preventing or treating nociceptive pain;

<2c> Use of a compound having a P2X4 receptor antagonistic action (for example, the compounds represented by the general formulas (A) to (BII)) or a pharmaceutically acceptable salt thereof for producing a medicament for preventing or treating visceral pain;

<3c> The use according to <1c> and <2c>, for producing a medicament for preventing or treating the nociceptive pain and the visceral pain;

<4c> The use according to <1c> or <3c>, in which the nociceptive pain is inflammatory pain;

<5c> The use according to <4c>, in which the inflammatory pain is pain in inflammatory bowel disease;

<6c> The use according to <2c> or <3c>, in which the visceral pain is visceral pain caused by the intestinal tract;

<7c> The use according to <6c>, in which the visceral pain caused by the intestinal tract is visceral pain caused by inflammatory bowel disease; and

<8c> The use according to any one of <2c>, <3c>, <6c>, and <7c>, in which the visceral pain is pain after remission or curing of inflammation.

### EXAMPLES

Hereinafter, the present invention is more specifically described with Examples, however, the scope of the present invention is not limited to the following Examples.

In the following Examples, as a P2X4 receptor antagonist, a 5-[3-(5-thioxo-4H-[1,2,4]oxadiazol-3-yl)phenyl]-1H-naphtho[1,2-b][1,4]diazepine-2,4(3H,5H)-dione sodium salt (a compound in Example 14 of WO 2010/093061 A: hereinafter, referred to as “Compound A”), 5-[4-(2-iodobenzoylamino)phenyl]-1H-naphtho[1,2-b][1,4]diazepine-2,4(3H,5H)-dione (a compound in Example 48 of WO 2013/105608 A: hereinafter, referred to as “Compound B”), and other compounds shown below were used:
a 5-[3-(1H-tetrazol-5-yl)phenyl]-1H-naphtho[1,2-b][1,4]diazepine-2,4(3H,5H)-dione sodium salt; 5-[4-[2-(trifluoromethyl)benzoyl]aminophenyl]-1H-naphtho[1,2-b][1,4]diazepine-2,4(3H,5H)-dione; 5-[4-[(2-chlorophenylacetyl)amino]phenyl]-1H-naphtho[1,2-b][1,4]diazepine-2,4(3H,5H)-dione; 1-[4-(2,4-dioxo-1,2,3,4-tetrahydronaphtho[1,2-b][1,4]diazepin-5-yl)phenyl]-3-phenylurea; 5-[4-[(2-ethylbenzoyl)amino]phenyl]-1H-naphtho[1,2-b][1,4]diazepine-2,4(3H,5H)-dione; 5-[4-(2-tert-butylbenzoylamino)phenyl]-1H-naphtho[1,2-b][1,4]diazepine-2,4(3H,5H)-dione; 5-[4-(2-iodobenzoyl)aminophenyl]-1,2,3,4,8,9,10,11-octahydronaphtho[1,2-b][1,4]diazepine-2,4(3H,5H)-dione; 5-[4-(6-chloro-2-hydroxybenzoylamino)phenyl]-1H-naphtho[1,2-b][1,4]diazepine-2,4(3H,5H)-dione; N-[4-(2,4-dioxo-1,2,3,4-tetrahydronaphtho[1,2-b][1,4]diazepin-5-yl)phenyl]benzenesulfonamide; 1-(2-chlorophenyl)-N-[4-(2,4-dioxo-1,2,3,4-tetrahydronaphtho[1,2-b][1,4]diazepin-5-yl)phenyl]methanesulfonamide; 1-(2-chlorophenyl)-N-[4-(2,4-dioxo-1,2,3,4,8,9,10,11-octahydronaphtho[1,2-b][1,4]diazepin-5-yl)phenyl]methanesulfonamide; 1-(2-bromophenyl)-N-[4-(2,4-dioxo-1,2,3,4-tetrahydronaphtho[1,2-b][1,4]diazepin-5-yl)phenyl]methanesulfonamide; N-[4-(2,4-dioxo-1,2,3,4-tetrahydronaphtho[1,2-b][1,4]diazepin-5-yl)phenyl]-1-(2-methylphenyl)methanesulfonamide; N-[4-(2,4-dioxo-1,2,3,4-tetrahydronaphtho[1,2-b][1,4]diazepin-5-yl)phenyl]-2-phenylethanesulfonamide; 1-(2-chlorophenyl)-N-[4-(2,4-dioxo-1,2,3,4,8,9,10,11-octahydronaphtho[1,2-b][1,4]diazepin-5-yl)phenyl]-N-methylmethanesulfonamide; 1-(2-chlorophenyl)-N-[4-(2-oxo-2,3-dihydro-1H-naphtho[1,2-e][1,4]diazepin-5-yl)phenyl]methanesulfonamide; 5-[4-[(2-methylpyridin-3-yl)carbonylamino]phenyl]-1H-naphtho[1,2-b][1,4]diazepine-2,4(3H,5H)-dione hydrochloride; 5-[4-[(2-chloropyridin-3-yl)carbonylamino]phenyl]-1H-naphtho[1,2-b][1,4]diazepine-2,4(3H,5H)-dione; 5-[4-[2-[(pyridin-2-yl)oxy]acetylamino]phenyl]-1H-naphtho[1,2-b][1,4]diazepine-2,4(3H,5H)-dione; and 5-[4-[(2-isopropylbenzoyl)amino]phenyl]-1H-naphtho[1,2-b][1,4]diazepine-2,4(3H,5H)-dione.

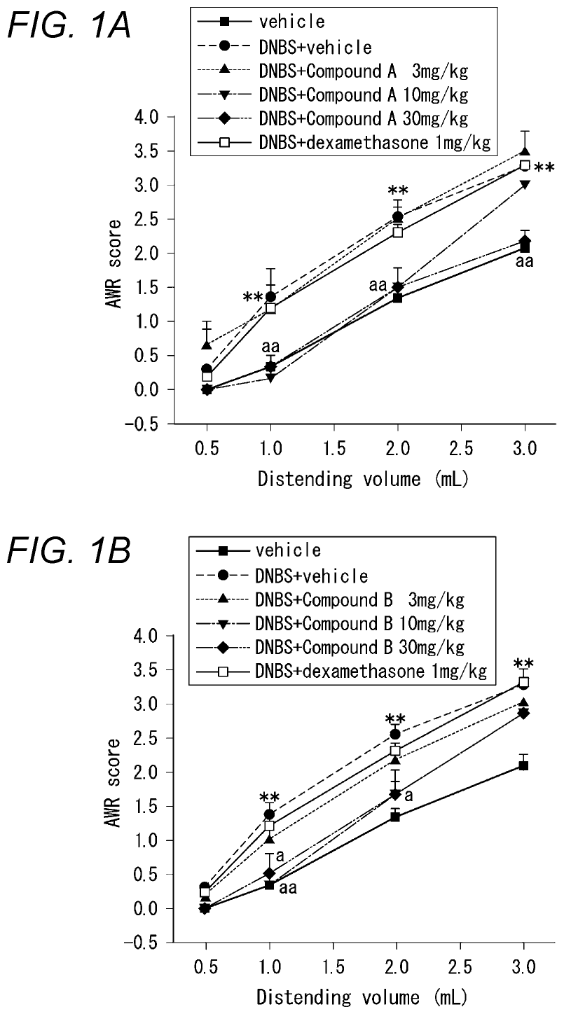

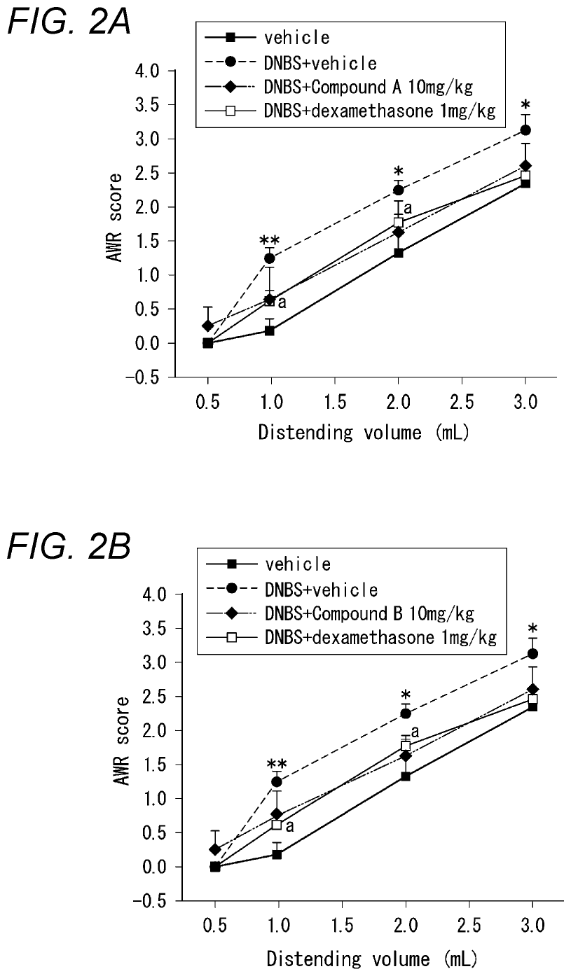

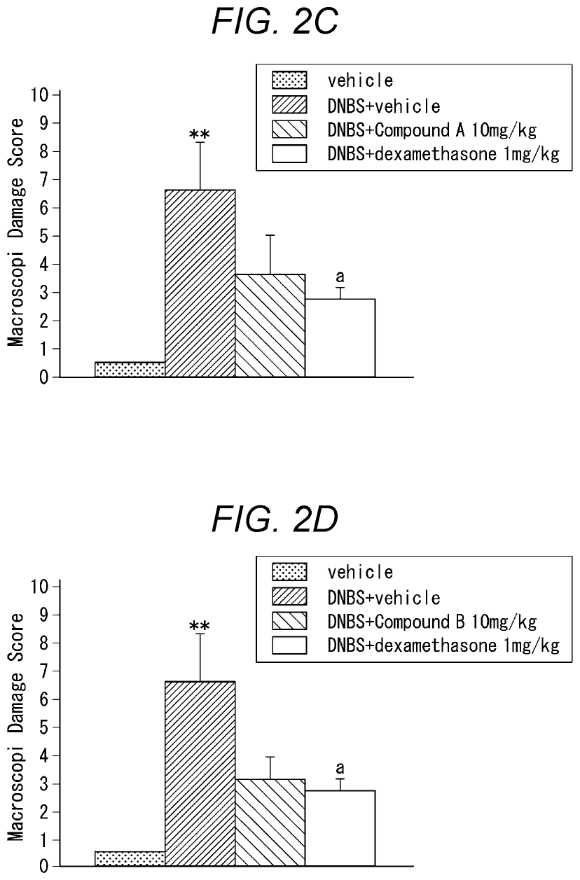

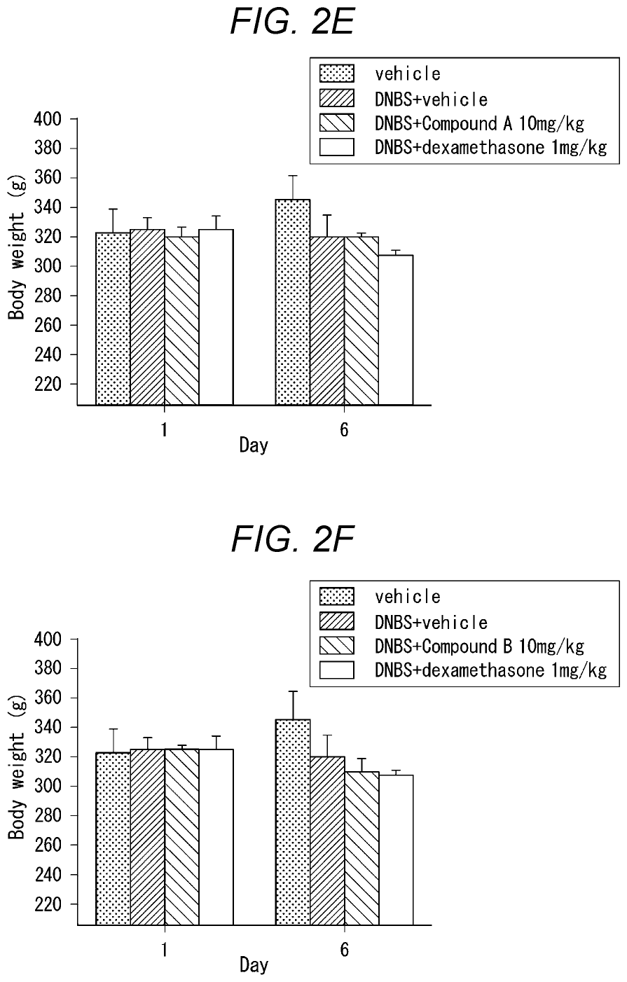

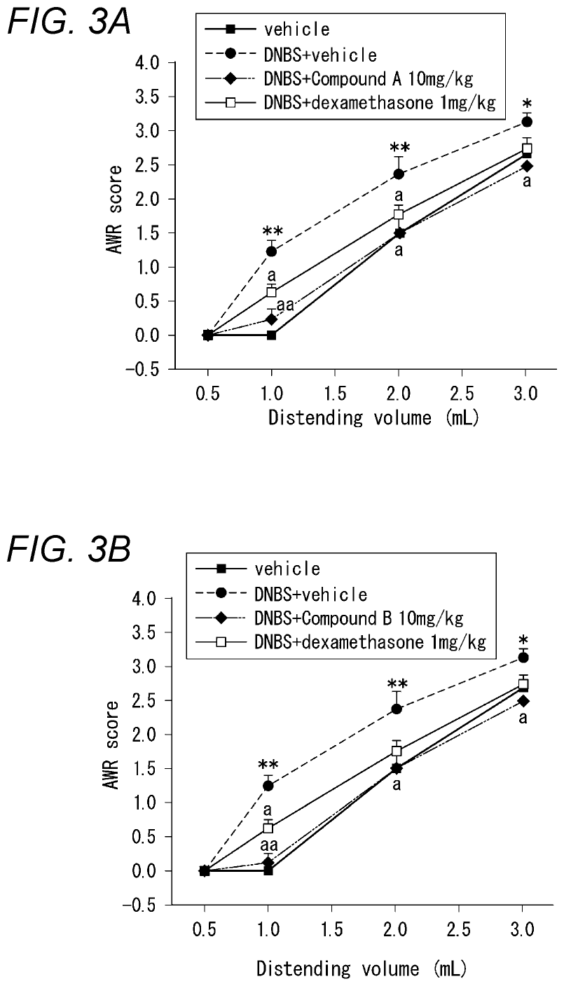

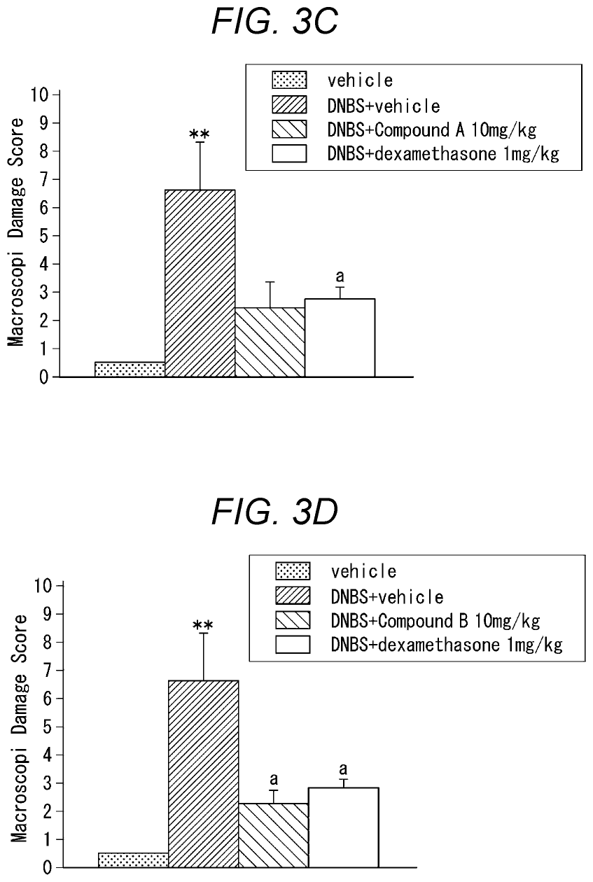

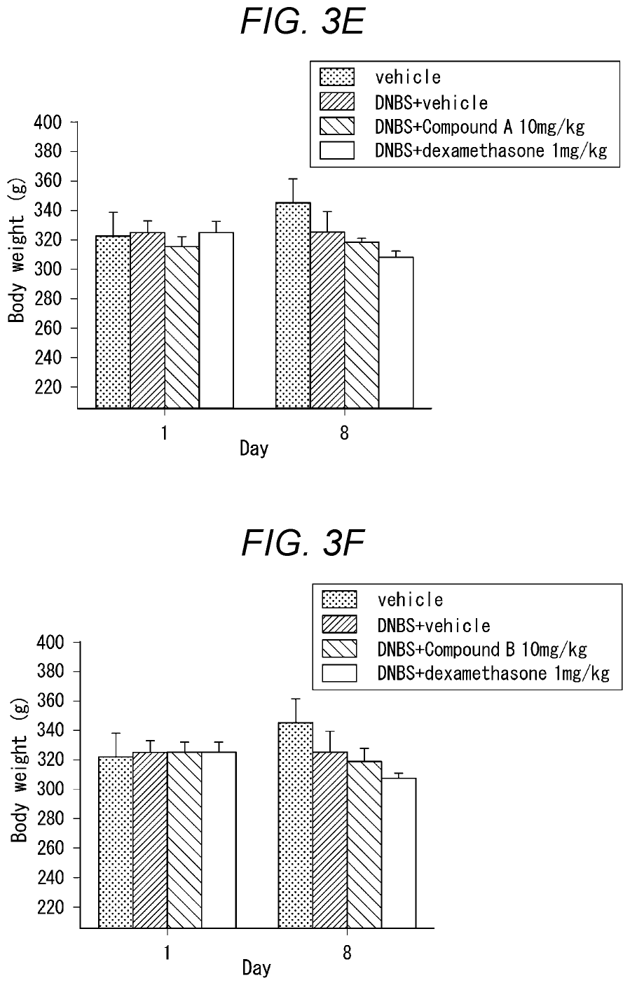

Note that, in Examples of the present application, significant differences are indicated with symbols “*” or “**” and “a” or “aa” in FIG. 1A, FIG. 1B, FIG. 2A to FIG. 2D, and FIG. 3A to FIG. 3D. “*” and “a” indicates that P<0.05, and “**” and “aa” indicates that P<0.01, respectively. In addition, the symbol “*” or “**” indicates that there is a significant difference as compared with control rats, and the symbol “a” or “aa” indicates that there is a significant difference as compared with a DNBS-induced colitis model.

### Example 1 (Measurement of P2X4 Receptor Antagonistic Action of Compound Which is Active Ingredient of Present Invention)

### (Test Method)

A P2X4 receptor antagonistic action of a compound which is the active ingredient of the present invention was measured. 1321N1 cells into which ATP receptors (human P2X4) were introduced were used as a P2X4 receptor stable expression system. The P2X4-receptor-expressing cells were seeded into a 96-well plate and cultured for 24 hours under a condition of 37° C. and 5% CO2 to be used for calcium measurement. Fura-2AM, which is a calcium fluorescent indicator, was dissolved in an extracellular fluid for calcium imaging, and the seeded cells were treated therewith and left to stand at room temperature for 45 minutes, thereby incorporating Fura-2AM into the cells. EnVision (PerkinElmer) which is a microplate reader was used for the measurement. 510 nm fluorescences F340 and F380 that are emitted when the cells are irradiated with light radiating from a xenon lamp and transmitted through 340 nm and 380 nm filters, respectively, were observed, and change in a ratio value F340/F380 was used as an index of intracellular calcium change. Measurement was performed by adding ATP to each well such that the final concentration of ATP becomes 1 μM and observing an ATP-induced intracellular calcium response over time. Inhibitory activity of a test substance was measured by performing pretreatment on the test substance for 15 minutes by adding ATP thereto, and the inhibitory activity was calculated by comparison with a case of absence of the test substance. The results are shown in the following Table 29.

### (Test Result)

TABLE 29

| Test substance | IC₅₀(μM) |
|---|---|
| Compound A2 | 0.53 |
| Compound A17 | 0.27 |
| (Compound A) | |
| Compound B2 | 0.75 |
| Compound B13 | 0.54 |
| Compound B20 | 1.2 |
| Compound B48 | 0.30 |
| (Compound B) | |
| Compound B57 | 0.72 |
| Compound B71 | 0.4 |
| Compound B106 | 1.8 |
| Compound B118 | 1.10 |
| Compound B146 | 0.67 |
| Compound B173 | 0.064 |
| Compound B177 | 0.056 |
| Compound B180 | 0.05 |
| Compound B181 | 0.068 |
| Compound B183 | 0.42 |
| Compound B196 | 0.97 |
| Compound B197 | 0.44 |
| Compound B208 | 1.3 |
| Compound B209 | 0.94 |
| Compound B210 | 1.4 |
| Compound B214 | 0.62 |

### Materials and Methods

### (Animals)

Male Sprague-Dawley rats (Envigo, Varese, Italy) weighing approximately 300 g at the beginning of a test were used. The animals were housed in Centro Stabulazione Animali da Laboratorio (CeSAL, University of Florence) and used at least one week after their arrival. Four rats were raised per cage (size 26×41 cm). The animals were fed with standard laboratory diet and tap water ad libitum and kept at 23±1° C. with a 12-hour light/dark cycle (light at 7:00 in the morning). All animal manipulations were carried out according to the European Community Guidelines for Animal Care (DL116/92) and the application of European Communities Council Directive of Nov. 24, 1986 (86/609/EEC). The ethical policy of the University of Florence complies with the Guide for the Care and Use of Laboratory Animals of the US National Institutes of Health (NIH Publication No. 85-23, revised 1996, University of Florence assurance number: A5278-01). Formal approval to conduct the experiments described was obtained from the Animal Subjects Review Board of the University of Florence. Experiments involving animals have been reported according to ARRIVE guidelines (McGrath and Lilley, Br. J. Pharmacol. 2015; 172:3189-3193). All efforts were made to minimize suffering of the animals and to reduce the number of animals used.

### (Induction of Colitis)

Colitis was induced by intrarectal administration of 15 mg of 2,4-dinitrobenzenesulfonic acid (DNBS; Sigma-Aldrich S.r.l., Milano, Italia) in 0.25 ml of 50% ethanol via a polyethylene PE-60 catheter inserted approximately 8 cm into the anus, while the rat was under short-term anesthesia by isoflurane (2%). To control rats, 0.25 ml of a vehicle was administered.

### (Administration of Medicament)

Compound A and Compound B were dissolved in water and orally administered 30 minutes before the beginning of a test, and an acute effect was evaluated.

In addition, in order to evaluate an effect of repeated administration of a P2X4 antagonist, two different tests (that is, prevention test and intervention test) were adopted. In the prevention test, the test medicament was administered daily to the rats immediately after the delivery of DNBS (Day 1), and the treatment was continued until the rats were sacrificed on the 6^{th} day (Day 6). In the intervention test, the daily treatment with the medicament began 2 days after the delivery of DNBS (Day 3), and the rats were sacrificed on the 8^{th} day (Day 8).

### (Evaluation of Visceral Sensitivity by Abdominal Withdrawal Reflex (AWR))

Visceral sensitivity to colorectal distension (CRD) was evaluated by measurement of abdominal withdrawal reflex (AWR) in conscious animals using semiquantitative scores (Lucarini et al., Cells. 2020; 9 (8): E1772). Briefly, the rats were anesthetized with isoflurane. A lubricated latex balloon (length: 4.5 cm) that is assembled into an embolectomy catheter by being attached to a polyethylene tube to be connected to a syringe filled with water was inserted into the rectum and the descending colon of adult rats through the anus. The tube was fixed to the tail by taping to hold the balloon in place. The rats were then allowed to recover from anesthesia for 15 minutes. The AWR measurement was performed by a blind observer with visual observation of responses of the animals to staged CRD (0.5, 1, 2, and 3 mL) which were assigned with the following scores:
no behavioral response to colorectal distension (0);
no movement during colorectal distension and occasional clinching of head at start of stimulation (1);
abdominal muscles slightly contract, however, abdomen is not lifted from platform (2);
strong contraction of abdominal muscles and lifting of abdomen from platform are observed (3); and
body arching and lifting of pelvic structure and scrotum are observed (4).

### (Histological Evaluation of Colonic Damage)

Evaluation of colonic damage was performed at a macroscopic level according to criteria previously reported by Antonioli et al. (Antonioli L et al., The Journal of pharmacology and experimental therapeutics 2010; 335:434-442).

The macroscopic criteria were as follows:
presence of adhesion between colon and another intraperitoneal organ (0-2: no adhesions: 0 points, adhesion with only one adjacent organ: 1 point, and adhesions with a plurality of adjacent organs: 2 points);
consistency of colon fecal matter (indirect marker of diarrhea; 0-2: formed stool: 0 points, loose stool: 1 point, and liquid stool: 2 points);
thickening of colonic wall (mm: 1 point was scored for 1 mm); and
presence and spread of hyperemia and macroscopic mucosal damage (0-5: none: 0 points, hyperemic: 1 point, ulcers without hyperemia or thickening of intestinal wall: 2 points, ulcer with inflammation at one site: 3 points, ulcers with inflammation at two or more sites: 4 points, and wide site of damage exceeding 1 cm along length of colon: 5 points).

### (Statistical Analysis)

Behavioral measurement was performed on five animals for each treatment performed in the two different test groups. All experimental procedures were performed by researchers who did not know about the treatment. Results were expressed as mean±S.E.M. Analysis of variance of behavioral data was performed by one-way analysis of variance using Bonferroni's significant difference procedure used for post-hoc comparison. Statistics of electrophysiological data were performed by Student's paired or unpaired t-test, or one-way analysis of variance followed by Bonferroni's post-hoc test, as appropriate. A P value below 0.05 was considered significant. Data were analyzed using the “Origin 9” software (OriginLab Corporation, Northampton, USA).

### Example 2 (Acute Administration Test)

### Effects of Acute Administration of P2X4 Antagonists Compound A and Compound B on Visceral Pain in Rats Induced by 2,4-Dinitrobenzenesulfonic Acid (DNBS)

FIG. 1 shows effects of acute administration of Compound A and Compound B on visceral pain (visceral hypersensitivity) in rats induced by intrarectal injection of DNBS (15 mg dissolved in 0.25 mL of EtOH 50%). Visceral sensitivity was evaluated by measuring abdominal withdrawal reflex (AWR) to colorectal distension (0.5 to 3 mL).

Seven days after the rectal injection, the DNBS-treated animals showed significantly higher abdominal responses to colorectal distension than the controls. The acute administration of Compound A (10 to 30 mg/kg) significantly and dose-dependently decreased the entity of AWR to colorectal distension in the DNBS-treated rats. A notable visceral pain threshold for the DNBS animals returned to a control value after administration of higher doses (FIG. 1A). Furthermore, the acute administration of Compound B (10 and 30 mg/kg) significantly reduced AWR to colorectal distension (1 to 2 mL).

Unlike Compounds A or B, dexamethasone 1 mg/kg did not affect visceral pain when acutely administered to the DNBS-treated animals.

### Example 3 (Prevention Test)

### Effects of Repeated Administration (Prevention Test) of P2X4 Antagonists Compound A and Compound B on Visceral Pain in Rats Induced by 2,4-Dinitrobenzenesulfonic Acid (DNBS)

FIG. 2 shows effects of the repeated administration (prevention test) of Compound A and Compound B on visceral pain (visceral hypersensitivity) and colonic damage in rats induced by DNBS injection. Visceral sensitivity was evaluated by measuring AWR to colorectal distension (0.5 to 3 mL). In the prevention test, the test medicament was administered daily to the rats immediately after the administration of DNBS, and the treatment was continued until the rats were sacrificed on the 6^{th} day (Day 6). The evaluation was performed 6 days after the induction of colitis and 24 hours after the last treatment with Compound A (A) and Compound B (B). After the test, the animals were sacrificed, and macroscopic damage scores were assigned to their colons (FIG. 2C and FIG. 2D). In FIG. 2E and FIG. 2F, weights (g) of the animals before the DNBS injection (Day 1) and at the end of the preventive treatment with Compound A and Compound B (Day 6) are shown, respectively.

The prevention test using Compound A or Compound B counteracted the colonic damage (FIG. 2C and FIG. 2D) and the onset of visceral pain (visceral hypersensitivity) occurring as a result of the DNBS injection (FIG. 2A and FIG. 2B). These effects were comparable to those exhibited by dexamethasone (1 mg/kg) with significant differences compared to the results from the DNBS+vehicle group (FIG. 2A to FIG. 2D). The administration of the P2X4 antagonists did not affect the weight loss caused by DNBS (approximately 10%, not significant; FIG. 2E and FIG. 2F). Notably, the administration of dexamethasone may contribute to the weight loss, as there is a reduction in muscle mass associated with sarcopenia which is one of the side effects of dexamethasone.

### Example 4 (Intervention Test)

### Effects of Repeated Administration (Intervention Test) of P2X4 Antagonists Compound A and Compound B on Visceral Pain in Rats Induced by 2,4-Dinitrobenzenesulfonic Acid (DNBS)

FIG. 3 shows effects of the repeated treatment with Compound A and Compound B in a case of applying the intervention test. In the test, the administration of the compounds began 2 days after the delivery of DNBS (Day 3) and was continued until the rats were sacrificed on the 8^{th} day (Day 8). Evaluation was performed 8 days after the induction of colitis (Day 8) and 24 hours after the last treatment with Compound A (A) and Compound B (B). After the test, the animals were sacrificed, and macroscopic damage scores were assigned to their colons (FIG. 3C and FIG. 3D). In FIG. 3E and FIG. 3F, weights (g) of the animals before the DNBS injection (Day 1) and at the end of the intervention treatment with Compound A and Compound B (Day 8) are reported, respectively.

Both the intervention treatment with Compound A and Compound B significantly reduces the onset of visceral pain (visceral hypersensitivity) occurring after the DNBS injection (FIG. 3A and FIG. 3B) and is more effective than the treatment with dexamethasone. The anti-hyperalgesia effect exhibited by these P2X4 antagonists, as well as dexamethasone, was accompanied by a significant reduction in colonic damage (FIGS. 3C and 3D). The administration of P2X4 antagonists does not affect weight loss caused by DNBS (approximately 10%, not significant; FIG. 3E and FIG. 3F). Notably, the administration of dexamethasone may contribute to the weight loss, as there is a reduction in muscle mass associated with sarcopenia which is one of the side effects of dexamethasone.

### Example 5 (Occludin Expression in Colonic Tissue)

An occludin (an integral plasma-membrane protein located at tight junctions) expression effect in the colonic tissues when the colons of the rats were subjected to DNBS-induced damage and the expression effect in the case of the administration of Compound A or Compound B were observed by western blotting.

### (Preparation of Model Animals to Be Used)

Sprague-Dawley rats (body weights of 200 to 225 g) which were male albino rats were used as model animals. Three model animals were housed in one cage in a temperature-controlled environment at 22-24° C., 50-60% humidity, and a light cycle of 12 hours, were given ad libitum access to standard laboratory feed and tap water, and were not used for at least one week after being brought into the laboratory.

The model animals were reared and handled in accordance with the provision of Directive 210/63/UE of the Council of the European Communities approved and adopted by the Italian government. The experiment was approved by the Ethics Committee on Animal Experimentation of the University of Pisa and the Italian Ministry of Health (authorization no. 674/2016-PR).

### (Preparation of DNBS-Induced Rats)

The procedures were performed according to the method described in Example 1.

### (Induction of Colitis)

The procedures were performed according to the method described in Example 1.

### (Sample Preparation)

The weight of the colonic tissue (30 μg of proteins) was measured, and the tissue was homogenized in a lysis buffer.

### (Test Method)

Proteins were quantified by the Bradford method. The proteins were separated with a precast 4-20% polyacrylamide gel (manufactured by Bio-Rad Laboratories, Inc., Mini-PROTEAN® TGX gel) and then transferred onto a PVDF membrane (manufactured by Bio-Rad Laboratories, Inc., Trans-Blot® Turbo™ PVDF

Transfer packs). The membrane was blocked with 3% BSA diluted with Tris-buffered saline (TBS, 20 mM Tris-HCl, pH 7.5, 150 mM NaCl) containing 0.1% Tween 20. An anti-occludin antibody (ab167161, manufactured by Abcam plc., dilution 1:5,000) was used as the antibody. Protein bands were detected using an ECL reagent (Clarity® Western ECL Blotting Substrate, Bio-Rad Laboratories, Inc.). Concentrations were measured with ImageJ software.

### (Observation of Occludin Expression)

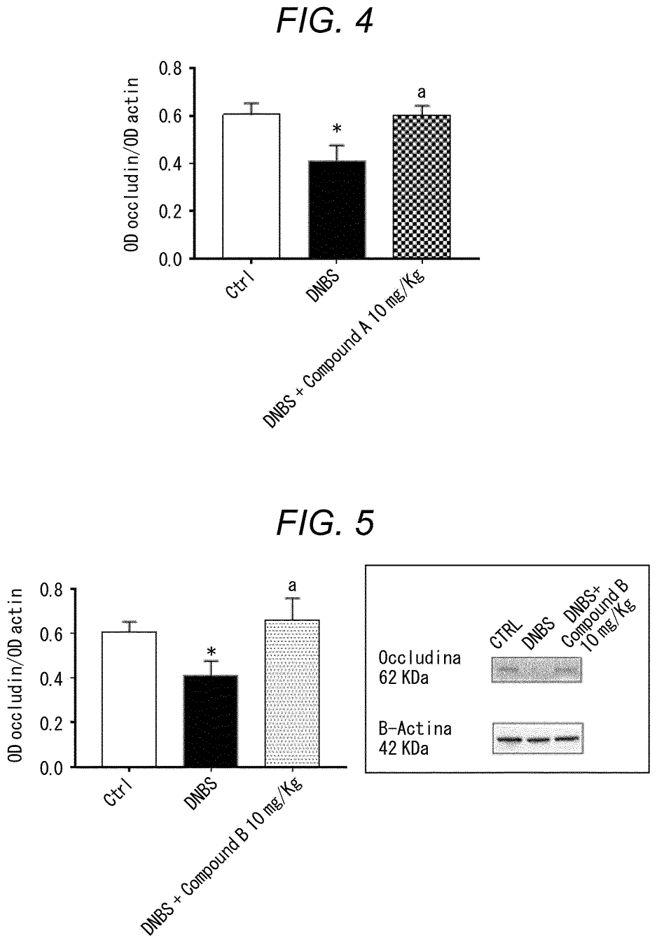

Colonic mucosa of the DNBS-induced rats showed significant decrease in occludin compared to the control rats (FIGS. 4 and 5).

The occludin expression increased in inflamed tissues of the groups administered with Compound A (10 mg/kg) (FIG. 4), Compound B (10 mg/kg) (FIG. 5), and dexamethasone (1 mg/kg, not shown in the drawing), compared to the DNBS-induced rats.

Alternatively, the occludin expression increased in inflamed tissues of the group administered with dexamethasone (1 mg/kg, not shown in the drawing), compared to the DNBS-induced rats.

From the above results, it was understood that Compound A and Compound B counteract the decrease in tight junction occludin observed in the DNBS-induced rats. Therefore, it was understood that Compound A and Compound B contribute to the function of maintaining health of the large intestinal tissue.

### INDUSTRIAL APPLICABILITY

The medicament of the present invention can be used for prevention or treatment of nociceptive pain, particularly, inflammatory pain.

1-11. (canceled)

12. A method of preventing or treating nociceptive pain, the method comprising administering an effective amount of a compound having a P2X4 receptor antagonistic action or a pharmaceutically acceptable salt thereof to a subject in need thereof.

13. A method of preventing or treating visceral pain, the method comprising administering an effective amount of a compound having a P2X4 receptor antagonistic action or a pharmaceutically acceptable salt thereof to a subject in need thereof.

14. A method of preventing or treating nociceptive pain and visceral pain, the method comprising administering an effective amount of a compound having a P2X4 receptor antagonistic action or a pharmaceutically acceptable salt thereof to a subject in need thereof.

15. The method according to claim 12, wherein the compound is a compound represented by the following the general formula (A): 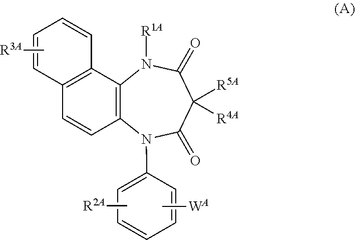
wherein, in the formula, R^{1A} represents a hydrogen atom, an alkyl group having 1 to 8 carbon atoms, an alkenyl group having 2 to 8 carbon atoms, an alkyl group having 1 to 8 carbon atoms substituted with 1 to 3 halogen atoms, or an alkyl group having 1 to 3 carbon atoms substituted with phenyl;
R^{2A} and R^{3A} may be the same or different, and represent a hydrogen atom, an alkyl group having 1 to 8 carbon atoms, an alkoxy group having 1 to 8 carbon atoms, an alkyl group having 1 to 8 carbon atoms substituted with 1 to 3 halogen atoms, an alkoxy group having 1 to 8 carbon atoms substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, an acylamino group having 2 to 8 carbon atoms substituted with 1 to 3 halogen atoms, an alkylsulfonylamino group having 1 to 8 carbon atoms, a carboxyl group, an acyl group having 2 to 8 carbon atoms, an alkoxycarbonyl group (the alkoxy moiety thereof has 1 to 8 carbon atoms), a carbamoyl group, an alkylthio group having 1 to 8 carbon atoms, an alkylsulfinyl group having 1 to 8 carbon atoms, an alkylsulfonyl group having 1 to 8 carbon atoms, or a sulfamoyl group;
R^{4A} and R^{5A} may be the same or different, and represent a hydrogen atom, an alkyl group having 1 to 8 carbon atoms, an alkyl group having 1 to 8 carbon atoms substituted with 1 to 3 halogen atoms, or an alkyl group having 1 to 3 carbon atoms substituted with phenyl; and
W^{A} represents a five or six membered heterocyclic ring comprising 1 to 4 nitrogen atoms as the members of the ring, which may have a substituent.

16. The method according to claim 12, wherein the compound is a compound represented by the following the general formula (BI): 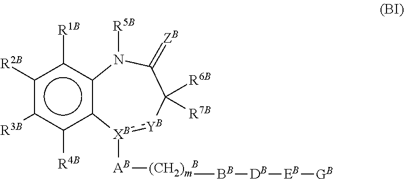
wherein, in the formula, R^{1B} and R^{2B} may be the same or different, and represent a hydrogen atom, an alkyl group having 1 to 8 carbon atoms, a cycloalkyl group having 3 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), a phenyl group which may be substituted, a pyridyl group which may be substituted, or an aralkyl group (the aryl moiety has 6 to 10 carbon atoms, and the alkylene moiety has 1 to 8 carbon atoms),
or
R^{1B} and R^{2B} may bind together to form a condensed ring selected from naphthalene ring, quinoline ring, isoquinoline ring, tetrahydronaphthalene ring, indane ring, tetrahydroquinoline ring, and tetrahydroisoquinoline ring together with the benzene ring to which they bind, and the ring constituted by R^{1B} and R^{2B}, bound to each other, together with the carbon atoms to which R^{1B} and R^{2B} bind may be substituted with 1 to 4 of the same or different substituents selected from an alkyl group having 1 to 8 carbon atoms, a cycloalkyl group having 3 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), and an aralkyl group (the aryl moiety has 6 to 10 carbon atoms, and the alkylene moiety has 1 to 8 carbon atoms),
R^{3B} and R^{4B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acylamino group having 2 to 8 carbon atoms, a carboxyl group, an acyl group having 2 to 8 carbon atoms, an alkoxycarbonyl group (the alkoxy moiety has 1 to 8 carbon atoms), or an aralkyl group (the aryl moiety has 6 to 10 carbon atoms, and the alkylene moiety has 1 to 8 carbon atoms),
R^{5B} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with hydroxyl group, or an aralkyl group (the aryl moiety has 6 to 10 carbon atoms, and the alkylene moiety has 1 to 8 carbon atoms),
R^{6B} and R^{7B} may be the same or different, and represent a hydrogen atom, an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, or an amino group,
X^{B} represents C, CH, or N,
Y^{B} represents N, NH, or C(═O),
provided that when X^{B} is N, Y^{B} is not N or NH, and
when X^{B} is C or CH, Y^{B} is not C(═O),
the double line consisting of the solid line and the broken line represents a single bond or a double bond,
Z^{B} represents oxygen atom, or sulfur atom,
A^{B} represents benzene ring, pyridine ring, thiophene ring, pyrimidine ring, naphthalene ring, quinoline ring, or indole ring, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an aralkyl group (the aryl moiety has 6 to 10 carbon atoms, and the alkylene moiety has 1 to 8 carbon atoms), phenyl group, and pyridyl group, as a substituent, or represents an atomic bond,
B^{B} represents N(R^{8B})C(═O), NHCONH, CON(R^{9B}), NHC(═S)NH, N(R^{10B})SO₂, SO₂N(R^{11B}), OSO₂, wherein
R^{8B}, R^{9B}, R^{10B}, and R^{11B} represent a hydrogen atom, an alkyl group having 1 to 8 carbon atoms, an alkyl group having 1 to 8 carbon atoms and substituted with 1 to 3 halogen atoms, an alkyl group having 1 to 8 carbon atoms and substituted with hydroxyl group, or an aralkyl group (the aryl moiety has 6 to 10 carbon atoms, and the alkylene moiety has 1 to 8 carbon atoms),
D^{B} represents an alkylene chain having 1 to 6 carbon atoms, which may have 1 to 4 of the same or different substituents selected from an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with hydroxyl group, and an aralkyl group (the aryl moiety has 6 to 10 carbon atoms, and the alkylene moiety has 1 to 8 carbon atoms), as a substituent, and may further have a double bond, or represents an atomic bond,
E^{B} represents O, S, NR^{12B}, or an atomic bond, wherein
R^{12B} represents a hydrogen atom, an alkyl group having 1 to 8 carbon atoms, an alkenyl group having 2 to 8 carbon atoms, an alkyl group having 1 to 8 carbon atoms and substituted with 1 to 3 halogen atoms, an alkyl group having 1 to 8 carbon atoms and substituted with hydroxyl group, or an aralkyl group (the aryl moiety has 6 to 10 carbon atoms, and the alkylene moiety has 1 to 8 carbon atoms),
G^{B} represents piperazine, piperidine, morpholine, cyclohexane, benzene, naphthalene, quinoline, quinoxaline, benzimidazole, thiophene, imidazole, thiazole, oxazole, indole, benzofuran, pyrrole, pyridine, or pyrimidine, which may have 1 to 4 of the same or different substituents selected from an alkyl group having 1 to 8 carbon atoms, an alkenyl group having 2 to 8 carbon atoms, an alkoxy group having 1 to 8 carbon atoms, an alkyl group having 1 to 8 carbon atoms and substituted with 1 to 3 halogen atoms, an alkoxy group having 1 to 8 carbon atoms and substituted with 1 to 3 halogen atoms, a halogen atom, a hydroxyl group, a nitro group, a cyano group, an amino group, an alkylamino group having 1 to 8 carbon atoms, a dialkylamino group having 2 to 8 carbon atoms, an acyl group having 2 to 8 carbon atoms, a methylenedioxy group, a carboxyl group, an alkylsulfinyl group having 1 to 6 carbon atoms, an alkylthio group having 1 to 6 carbon atoms, an alkylsulfonyl group having 1 to 6 carbon atoms, an aralkyl group (the aryl moiety has 6 to 10 carbon atoms, and the alkylene moiety has 1 to 8 carbon atoms), a phenyl group which may be substituted, a pyridyl group which may be substituted, an imidazolyl group which may be substituted, an oxazolyl group which may be substituted, and a thiazolyl group which may be substituted, as a substituent, and
m^{B} represents an integer of 0 to 5,
provided that when R^{1B} and R^{2B} do not bind together to form a ring, those compounds are excluded wherein, X is C, Y is N, the double line consisting of the solid line and the broken line is a double bond, Z is oxygen atom, A is benzene ring, m is 0, B is C(═O)NH, E is an atomic bond, and G is phenyl group.

17. The method according to claim 12, wherein the compound or pharmaceutically acceptable salt thereof, is selected from the group consisting of the following (A1) to (A21) and (B1) to (B214):
(A1) 5-[3-(1H-tetrazol-5-yl)phenyl]-1H-naphtho[1,2-b][1,4]diazepine-2,4(3H,5H)-dione;
(A2) 5-[3-(1H-tetrazol-5-yl)phenyl]-1H-naphtho[1,2-b][1,4]diazepine-2,4(3H,5H)-dione sodium salt;
(A3) 5-[3-(1H-tetrazol-5-yl)phenyl]-1H-naphtho[1,2-b][1,4]diazepine-2,4(3H,5H)-dione potassium salt;
(A4) 5-[4-(1H-tetrazol-5-yl)phenyl]-1H-naphtho[1,2-b][1,4]diazepine-2,4(3H,5H)-dione;
(A5) 5-[4-(1H-tetrazol-5-yl)phenyl]-1H-naphtho[1,2-b][1,4]diazepine-2,4(3H,5H)-dione sodium salt;
(A6) 1-Methyl-5-[3-(1H-tetrazol-5-yl)phenyl]-1H-naphtho[1,2-b][1,4]diazepine-2,4(3H,5H)-dione;
(A7) 1,3-dimethyl-5-[3-(1H-tetrazol-5-yl)phenyl]-1H-naphtho[1,2-b][1,4]diazepine-2,4(3H,5H)-dione;
(A8) 5-[2-chloro-5-(1H-tetrazol-5-yl)phenyl]-1H-naphtho[1,2-b][1,4]diazepine-2,4(3H,5H)-dione;
(A9) 5-[2-chloro-5-(1H-tetrazol-5-yl)phenyl]-1H-naphtho[1,2-b][1,4]diazepine-2,4(3H,5H)-dione sodium salt;
(A10) 5-[2-methyl-5-(1H-tetrazol-5-yl)phenyl]-1H-naphtho[1,2-b][1,4]diazepine-2,4(3H,5H)-dione;
(A11) 5-[2-methyl-5-(1H-tetrazol-5-yl)phenyl]-1H-naphtho[1,2-b][1,4]diazepine-2,4(3H,5H)-dione sodium salt;
(A12) 5-[2-bromo-5-(1H-tetrazol-5-yl)phenyl]-1H-naphtho[1,2-b][1,4]diazepine-2,4(3H,5H)-dione;
(A13) 5-[3-(2-methyl-2H-tetrazol-5-yl)phenyl]-1H-naphtho[1,2-b][1,4]diazepine-2,4(3H,5H)-dione;
(A14) 5-[3-(1-methyl-1H-tetrazol-5-yl)phenyl]-1H-naphtho[1,2-b][1,4]diazepine-2,4(3H,5H)-dione;
(A15) 5-[3-(5-oxo-4H-[1,2,4]oxadiazol-3-yl)phenyl]-1H-naphtho[1,2-b][1,4]diazepine-2,4(3H,5H)-dione;
(A16) 5-[3-(5-thioxo-4H-[1,2,4]oxadiazol-3-yl)phenyl]-1H-naphtho[1,2-b][1,4]diazepine-2,4(3H,5H)-dione;
(A17) 5-[3-(5-thioxo-4H-[1,2,4]oxadiazol-3-yl)phenyl]-1H-naphtho[1,2-b][1,4]diazepine-2,4(3H,5H)-dione sodium salt;
(A18) 5-[3-(oxazol-2-yl)phenyl]-1H-naphtho[1,2-b][1,4]diazepine-2,4(3H,5H)-dione;
(A19) 5-[3-(1H-pyrazol-4-yl)phenyl]-1H-naphtho[1,2-b][1,4]diazepine-2,4(3H,5H)-dione;
(A20) 5-[4-fluoro-3-(1H-tetrazol-5-yl)phenyl]-1H-naphtho[1,2-b][1,4]diazepine-2,4(3H,5H)-dione;
(A21) 5-[4-fluoro-3-(1H-tetrazol-5-yl)phenyl]-1H-naphtho[1,2-b][1,4]diazepine-2,4(3H,5H)-dione sodium salt;
(B1) 5-(4-benzoylaminophenyl)-1H-naphtho[1,2-b][1,4]diazepine-2,4(3H,5H)-dione;
(B2) 5-[4-[2-(trifluoromethyl)benzoyl]aminophenyl]-1H-naphtho[1,2-b][1,4]diazepine-2,4(3H,5H)-dione;
(B3) 5-[4-(3-bromobenzoyl)aminophenyl]-1H-naphtho[1,2-b][1,4]diazepine-2,4(3H,5H)-dione
(B4) 5-[4-[4-(trifluoromethyl)benzoyl]aminophenyl]-1H-naphtho[1,2-b][1,4]diazepine-2,4(3H,5H)-dione;
(B5) 5-[4-(2-methylbenzoyl)aminophenyl]-1H-naphtho[1,2-b][1,4]diazepine-2,4(3H,5H)-dione;
(B6) 5-[4-(2,6-dDimethylbenzoylamino)phenyl]-1H-naphtho[1,2-b][1,4]diazepine-2,4(3H,5H)-dione;
(B7) 5-[4-(2,6-dichlorobenzoyl)aminophenyl]-1H-naphtho[1,2-b][1,4]diazepine-2,4(3H,5H)-dione;
(B8) 5-[4-(3-chlorobenzoyl)aminophenyl]-1H-naphtho[1,2-b][1,4]diazepine-2,4(3H,5H)-dione;
(B9) 5-[4-(2-phenylacetylamino)phenyl]-1H-naphtho[1,2-b][1,4]diazepine-2,4(3H,5H)-dione;
(B10) 1-[4-(2,4-dioxo-1,2,3,4-tetrahydronaphtho[1,2-b][1,4]diazepin-5-yl)phenyl]-3-phenylthiourea;
(B11) 5-[4-(2,3-dimethoxybenzoylamino)phenyl]-1H-naphtho[1,2-b][1,4]diazepine-2,4(3H,5H)-dione;
(B12) 5-[4-(2-methoxybenzoylamino)phenyl]-1H-naphtho[1,2-b][1,4]diazepine-2,4(3H,5H)-dione;
(B13) 5-[4-[(2-chlorophenylacetyl)amino]phenyl]-1H-naphtho[1,2-b][1,4]diazepine-2,4(3H,5H)-dione;
(B14) 5-[4-(2,3-dimethylbenzoylamino)phenyl]-1H-naphtho[1,2-b][1,4]diazepine-2,4(3H,5H)-dione;
(B15) 5-[4-(2,5-dimethylbenzoylamino)phenyl]-1H-naphtho[1,2-b][1,4]diazepine-2,4(3H,5H)-dione;
(B16) 5-[4-(5-bromo-2-chlorobenzoylamino)phenyl]-1H-naphtho[1,2-b][1,4]diazepine-2,4(3H,5H)-dione;
(B17) 5-[4-(2,4-dichlorobenzoylamino)phenyl]-1H-naphtho[1,2-b][1,4]diazepine-2,4(3H,5H)-dione;
(B18) 5-[4-(2-hydroxybenzoylamino)phenyl]-1H-naphtho[1,2-b][1,4]diazepine-2,4(3H,5H)-dione;
(B19) 5-[4-(2,3-dihydroxybenzoylamino)phenyl]-1H-naphtho[1,2-b][1,4]diazepine-2,4(3H,5H)-dione;
(B20) 1-[4-(2,4-dioxo-1,2,3,4-tetrahydronaphtho[1,2-b][1,4]diazepin-5-yl)phenyl]-3-phenylurea;
(B21) 5-[4-[(2,6-dichlorophenylacetyl)amino]phenyl]-1H-naphtho[1,2-b][1,4]diazepine-2,4(3H,5H)-dione;
(B22) 5-[4-[(2-methoxyphenylacetyl)amino]phenyl]-1H-naphtho[1,2-b][1,4]diazepine-2,4(3H,5H)-dione;
(B23) 5-[4-[(2-hydroxyphenylacetyl)amino]phenyl]-1H-naphtho[1,2-b][1,4]diazepine-2,4(3H,5H)-dione;
(B24) 1-(2-chlorophenyl)-3-[4-(2,4-dioxo-1,2,3,4-tetrahydronaphtho[1,2-b][1,4]diazepin-5-yl)phenyl]thiourea;
(B25) 5-[4-[3-(trifluoromethyl)benzoylamino]phenyl]-1H-naphtho[1,2-b][1,4]diazepine-2,4(3H,5H)-dione;
(B26) 5-[4-[2-[(2-trifluoromethyl)phenyl]acetylamino]phenyl]-1H-naphtho[1,2-b][1,4]diazepine-2,4(3H,5H)-dione;
(B27) 1-(2-chlorophenyl)-3-[4-(2,4-dioxo-1,2,3,4-tetrahydronaphtho[1,2-b][1,4]diazepin-5-yl)phenyl]urea;
(B28) 5-[4-[(2-phenylpropionyl)amino]phenyl]-1H-naphtho[1,2-b][1,4]diazepine-2,4(3H,5H)-dione;
(B29) 5-[4-(2-chloro-3-methoxybenzoylamino)phenyl]-1H-naphtho[1,2-b][1,4]diazepine-2,4(3H,5H)-dione;
(B30) 5-[4-(3-phenylpropionylamino)phenyl]-1H-naphtho[1,2-b][1,4]diazepine-2,4(3H,5H)-dione;
(B31) 5-[4-[(1H-indole-3-carbonyl)amino]phenyl]-1H-naphtho[1,2-b][1,4]diazepine-2,4(3H,5H)-dione;
(B32) 5-[4-(2-chloro-3-hydroxybenzoylamino)phenyl]-1H-naphtho[1,2-b][1,4]diazepine-2,4(3H,5H)-dione;
(B33) 5-[4-[(2-methyl-2-phenylpropionyl)amino]phenyl]-1H-naphtho[1,2-b][1,4]diazepine-2,4(3H,5H)-dione;
(B34) 5-[4-(2-phenoxyacetylamino)phenyl]-1H-naphtho[1,2-b][1,4]diazepine-2,4(3H,5H)-dione;
(B35) 5-[4-[2-(2-chloro-4-methoxyphenyl)acetylamino]phenyl]-1H-naphtho[1,2-b][1,4]diazepine-2,4(3H,5H)-dione;
(B36) 5-[4-[(1-methyl-1H-imidazole-2-carbonyl)amino]phenyl]-1H-naphtho[1,2-b][1,4]diazepine-2,4(3H,5H)-dione;
(B37) 5-[4-[2-(2,4-dichlorophenyl)acetylamino]phenyl]-1H-naphtho[1,2-b][1,4]diazepine-2,4(3H,5H)-dione;
(B38) 5-[4-[2-(2-chloro-4-hydroxyphenyl)acetylamino]phenyl]-1H-naphtho[1,2-b][1,4]diazepine-2,4(3H,5H)-dione;
(B39) 5-[4-(3-phenylpropenylamino)phenyl]-1H-naphtho[1,2-b][1,4]diazepine-2,4(3H,5H)-dione;
(B40) 5-[4-[(3-pyridylacetyl)amino]phenyl]-1H-naphtho[1,2-b][1,4]diazepine-2,4(3H,5H)-dione hydrochloride;
(B41) 5-[4-(1H-benzimidazole-2-carbonylamino)phenyl]-1H-naphtho[1,2-b][1,4]diazepine-2,4(3H,5H)-dione;
(B42) 1-[4-(2,3-dimethylbenzoylamino)phenyl]-7-methoxy-1H-1,5-benzodiazepine-2,4(3H,5H)-dione;
(B43) 5-[4-[(benzoylamino)methyl]phenyl]-1H-naphtho[1,2-b][1,4]diazepine-2,4(3H,5H)-dione;
(B44) 5-[4-[(2-chlorobenzoylamino)methyl]phenyl]-1H-naphtho[1,2-b][1,4]diazepine-2,4(3H,5H)-dione;
(B45) 1-[4-(2,3-dimethylbenzoylamino)phenyl]-7-hydroxy-1H-1,5-benzodiazepine-2,4(3H,5H)-dione;
(B46) 5-[4-(2-chlorobenzoylamino)phenyl]-1H-naphtho[1,2-b][1,4]diazepine-2,4(3H,5H)-dione;
(B47) 5-[4-(2-bromobenzoylamino)phenyl]-1H-naphtho[1,2-b][1,4]diazepine-2,4(3H,5H)-dione;
(B48) 5-[4-(2-iodobenzoylamino)phenyl]-1H-naphtho[1,2-b][1,4]diazepine-2,4(3H,5H)-dione;
(B49) 5-[4-(2,3-dimethylbenzoylamino)-3-fluorophenyl]-1H-naphtho[1,2-b][1,4]diazepine-2,4(3H,5H)-dione;
(B50) 5-[4-[2-(2-methylphenyl)acetylamino]phenyl]-1H-naphtho[1,2-b][1,4]diazepine-2,4(3H,5H)-dione;
(B51) 5-[4-[(quinoxalin-2-yl)carbonylamino]phenyl]-1H-naphtho[1,2-b][1,4]diazepine-2,4(3H,5H)-dione;
(B52) 5-[4-[(5-methylthiophen-2-yl)carbonylamino]phenyl]-1H-naphtho[1,2-b][1,4]diazepine-2,4(3H,5H)-dione;
(B53) 5-[3-[(2-chlorophenylacetyl)amino]phenyl]-1H-naphtho[1,2-b][1,4]diazepine-2,4(3H,5H)-dione;
(B54) 5-[4-[(2,4,6-trimethylbenzoyl)amino]phenyl]-1H-naphtho[1,2-b][1,4]diazepine-2,4(3H,5H)-dione;
(B55) 5-[4-(cyclohexylcarbonylamino)phenyl]-1,2,3,4,8,9,10,11-octahydronaphtho[1,2-b][1,4]diazepine-2,4(3H,5H)-dione;
(B56) 1-[4-(2,3-dimethylbenzoyl)aminophenyl]-6-methyl-1H-1,5-benzodiazepine-2,4(3H,5H)-dione;
(B57) 5-[4-[(2-ethylbenzoyl)amino]phenyl]-1H-naphtho[1,2-b][1,4]diazepine-2,4(3H,5H)-dione;
(B58) 5-[4-[(6-methylpyridin-2-yl)carbonylamino]phenyl]-1,2,3,4,8,9,10,11-octahydronaphtho[1,2-b][1,4]diazepine-2,4(3H,5H)-dione;
(B59) 5-[4-[(2-methylpyridin-3-yl)carbonylamino]phenyl]-1,2,3,4,8,9,10,11-octahydronaphtho[1,2-b][1,4]diazepine-2,4(3H,5H)-dione;
(B60) 1-[4-(2,4-dioxo-1,2,3,4-tetrahydronaphtho[1,2-b][1,4]diazepin-5-yl)phenyl]-3-(2-methylphenyl)thiourea;
(B61) 5-[4-(2-methoxy-3-methylbenzoyl)aminophenyl]-1,2,3,4,8,9,10,11-octahydronaphtho[1,2-b][1,4]diazepine-2,4(3H,5H)-dione;
(B62) 5-[4-(2,3-dichlorobenzoyl)aminophenyl]-1,2,3,4,8,9,10,11-octahydronaphtho[1,2-b][1,4]diazepine-2,4(3H,5H)-dione;
(B63) 5-[4-(2,3-dimethylbenzoylamino)-3-hydroxyphenyl]-1H-naphtho[1,2-b][1,4]diazepine-2,4(3H,5H)-dione;
(B64) 5-[4-(2-chloro-3-methoxybenzoylamino)phenyl]-1,3-dihydronaphtho[1,2-e]-1,4-diazepin-2-one;
(B65) 5-[4-[(4-dimethylaminobenzoyl)amino]phenyl]-1H-naphtho[1,2-b][1,4]diazepine-2,4(3H,5H)-dione;
(B66) 5-[4-[2-(2,4-dichlorophenoxy)acetylamino]phenyl]-1H-naphtho[1,2-b][1,4]diazepine-2,4(3H,5H)-dione;
(B67) 5-[4-[2-(2-methylphenoxy)acetylamino]phenyl]-1H-naphtho[1,2-b][1,4]diazepine-2,4(3H,5H)-dione;
(B68) N-[4-(2,4-dioxo-1,2,3,4-tetrahydronaphtho[1,2-b][1,4]diazepin-5-yl)butyl]-2-chloro-3-methoxybenzamide;
(B69) 5-[4-(2-chloro-3-hydroxybenzoylamino)phenyl]-1,3-dihydronaphtho[1,2-e]-1,4-diazepin-2-one;
(B70) 5-[4-(2-acetylbenzoylamino)phenyl]-1H-naphtho[1,2-b][1,4]diazepine-2,4(3H,5H)-dione;
(B71) 5-[4-(2-tert-butylbenzoylamino)phenyl]-1H-naphtho[1,2-b][1,4]diazepine-2,4(3H,5H)-dione;
(B72) 5-[2-(2-iodobenzoyl)aminoethyl]-1H-naphtho[1,2-b][1,4]diazepine-2,4(3H,5H)-dione;
(B73) 5-[3-[(2-iodobenzoyl)amino]phenyl]-1H-naphtho[1,2-b][1,4]diazepine-2,4(3H,5H)-dione;
(B74) 6,7-dimethyl-1-[4-(2-iodobenzoyl)aminophenyl]-1H-1,5-benzodiazepine-2,4(3H,5H)-dione;
(B75) 5-[4-[(1-methylpiperidin-4-yl)carbonylamino]phenyl]-1H-naphtho[1,2-b][1,4]diazepine-2,4(3H,5H)-dione hydrochloride;
(B76) 5-[4-[(benzofuran-2-yl)carbonylamino]phenyl]-1H-naphtho[1,2-b][1,4]diazepine-2,4(3H,5H)-dione;
(B77) 5-[4-[(1-methyl-1H-indol-3-yl)carbonylamino]phenyl]-1H-naphtho[1,2-b][1,4]diazepine-2,4(3H,5H)-dione;
(B78) 5-[4-(2-propenylbenzoylamino)phenyl]-1H-naphtho[1,2-b][1,4]diazepine-2,4(3H,5H)-dione;
(B79) 5-[4-(2-propylbenzoylamino)phenyl]-1H-naphtho[1,2-b][1,4]diazepine-2,4(3H,5H)-dione;
(B80) 5-[3-fluoro-4-(2-iodobenzoylamino)phenyl]-1H-naphtho[1,2-b][1,4]diazepine-2,4(3H,5H)-dione;
(B81) 5-[4-(2-hydroxy-3-methylbenzoyl)aminophenyl]-1,2,3,4,8,9,10,11-octahydronaphtho[1,2-b][1,4]diazepine-2,4(3H,5H)-dione;
(B82) 5-[4-[(2-isopropoxybenzoyl)amino]phenyl]-1H-naphtho[1,2-b][1,4]diazepine-2,4(3H,5H)-dione;
(B83) 5-[4-[(3-methylthiophen-2-yl)carbonylamino]phenyl]-1H-naphtho[1,2-b][1,4]diazepine-2,4(3H,5H)-dione;
(B84) 5-[4-(2-phenoxypropionylamino)phenyl]-1H-naphtho[1,2-b][1,4]diazepine-2,4(3H,5H)-dione;
(B85) 5-[4-[2-(4-chloro-2-methylphenoxy)acetylamino]phenyl]-1H-naphtho[1,2-b][1,4]diazepine-2,4(3H,5H)-dione;
(B86) 5-[4-(4-fluoro-2-trifluoromethylbenzoyl)aminophenyl]-1H-naphtho[1,2-b][1,4]diazepine-2,4(3H,5H)-dione;
(B87) 5-[4-(4-fluoro-2-methoxybenzoyl)aminophenyl]-1H-naphtho[1,2-b][1,4]diazepine-2,4(3H,5H)-dione;
(B88) 5-[4-(4-fluoro-2-hydrooxybenzoyl)aminophenyl]-1H-naphtho[1,2-b][1,4]diazepine-2,4(3H,5H)-dione;
(B89) 5-[3-[(2-iodophenylacetyl)amino]phenyl]-1H-naphtho[1,2-b][1,4]diazepine-2,4(3H,5H)-dione;
(B90) 5-[4-(2-methyl-2-phenoxypropionylamino)phenyl]-1H-naphtho[1,2-b][1,4]diazepine-2,4(3H,5H)-dione;
(B91) 5-[4-(2-tert-butylbenzoylamino)phenyl]-1,3-dihydronaphtho[1,2-e]-1,4-diazepin-2-one;
(B92) 5-[4-[(3-dimethylaminobenzoyl)amino]phenyl]-1H-naphtho[1,2-b][1,4]diazepine-2,4(3H,5H)-dione;
(B93) 5-[4-(4-iodo-2-methoxybenzoylamino)phenyl]-1H-naphtho[1,2-b][1,4]diazepine-2,4(3H,5H)-dione;
(B94) 5-[4-(6-fluoro-2-methoxybenzoylamino)phenyl]-1H-naphtho[1,2-b][1,4]diazepine-2,4(3H,5H)-dione;
(B95) 5-[4-(2-hydroxy-4-iodobenzoylamino)phenyl]-1H-naphtho[1,2-b][1,4]diazepine-2,4(3H,5H)-dione;
(B96) 5-[4-(6-fluoro-2-hydroxybenzoylamino)phenyl]-1H-naphtho[1,2-b][1,4]diazepine-2,4(3H,5H)-dione;
(B97) 5-[4-(2-fluorobenzoyl)aminophenyl]-1H-naphtho[1,2-b][1,4]diazepine-2,4(3H,5H)-dione;
(B98) 5-[4-[(2-dimethylaminobenzoyl)amino]phenyl]-1H-naphtho[1,2-b][1,4]diazepine-2,4(3H,5H)-dione;
(B99) 5-[4-(2-methoxy-6-methylbenzoylamino)phenyl]-1H-naphtho[1,2-b][1,4]diazepine-2,4(3H,5H)-dione;
(B100) 5-[4-(2-hydroxy-6-methylbenzoylamino)phenyl]-1H-naphtho[1,2-b][1,4]diazepine-2,4(3H,5H)-dione;
(B101) 5-[4-[3-(2-methylphenyl)propionylamino]phenyl]-1H-naphtho[1,2-b][1,4]diazepine-2,4(3H,5H)-dione;
(B102) 5-(4-phenylcarbamoylphenyl)-1H-naphtho[1,2-b][1,4]diazepine-2,4(3H,5H)-dione;
(B103) 5-(4-benzylcarbamoylphenyl)-1H-naphtho[1,2-b][1,4]diazepine-2,4(3H,5H)-dione;
(B104) 5-[4-[3-(2-methylphenyl)propenoylamino]phenyl]-1H-naphtho[1,2-b][1,4]diazepine-2,4(3H,5H)-dione;
(B105) 5-[4-[3-(2-chlorophenyl)propionylamino]phenyl]-1H-naphtho[1,2-b][1,4]diazepine-2,4(3H,5H)-dione;
(B106) 5-[4-(2-iodobenzoyl)aminophenyl]-1,2,3,4,8,9,10,11-octahydronaphtho[1,2-b][1,4]diazepine-2,4(3H,5H)-dione;
(B107) 5-[4-[(1-methyl-1H-pyrrol-2-ylacetyl)amino]phenyl]-1,2,3,4,8,9,10,11-octahydronaphtho[1,2-b][1,4]diazepine-2,4(3H,5H)-dione;
(B108) 5-[4-(2-chlorobenzyl)carbamoylphenyl]-1H-naphtho[1,2-b][1,4]diazepine-2,4(3H,5H)-dione;
(B109) 5-[4-[3-(2-chlorophenyl)propenoylamino]phenyl]-1H-naphtho[1,2-b][1,4]diazepine-2,4(3H,5H)-dione;
(B110) 5-[4-(2-chlorophenyl)carbamoylphenyl]-1H-naphtho[1,2-b][1,4]diazepine-2,4(3H,5H)-dione;
(B111) 5-[4-(6-bromo-2,3-methylenedioxybenzoylamino)phenyl]-1H-naphtho[1,2-b][1,4]diazepine-2,4(3H,5H)-dione;
(B112) 5-[4-(6-bromo-2-methoxybenzoylamino)phenyl]-1H-naphtho[1,2-b][1,4]diazepine-2,4(3H,5H)-dione;
(B113) 5-[4-[(2-tert-butylbenzoyl)amino]phenyl]-1,2,3,4,8,9,10,11-octahydronaphtho[1,2-b][1,4]diazepine-2,4(3H,5H)-dione;
(B114) 5-[2-(2-iodobenzoyl)aminopyridin-5-yl]-1H-naphtho[1,2-b][1,4]diazepine-2,4(3H,5H)-dione;
(B115) 5-[4-(6-bromo-2-hydroxybenzoylamino)phenyl]-1H-naphtho[1,2-b][1,4]diazepine-2,4(3H,5H)-dione;
(B116) 5-[4-(6-chloro-2-methoxybenzoylamino)phenyl]-1H-naphtho[1,2-b][1,4]diazepine-2,4(3H,5H)-dione;
(B117) 5-[4-(2-iodobenzoylamino)phenyl]-1H-[1,4]diazepino[2,3-h]quinoline-2,4(3H,5H)-dione;
(B118) 5-[4-(6-chloro-2-hydroxybenzoylamino)phenyl]-1H-naphtho[1,2-b][1,4]diazepine-2,4(3H,5H)-dione;
(B119) 5-[4-(2-hydroxy-6-methoxybenzoylamino)phenyl]-1H-naphtho[1,2-b][1,4]diazepine-2,4(3H,5H)-dione;
(B120) 5-[4-[2-methoxy-6-(trifluoromethyl)benzoylamino]phenyl]-1H-naphtho[1,2-b][1,4]diazepine-2,4(3H,5H)-dione;
(B121) 5-[4-[2-hydroxy-6-(trifluoromethyl)benzoylamino]phenyl]-1H-naphtho[1,2-b][1,4]diazepine-2,4(3H,5H)-dione;
(B122) 5-[4-[(2-isopropenylbenzoyl)amino]phenyl]-1,2,3,4,8,9,10,11-octahydronaphtho[1,2-b][1,4]diazepine-2,4(3H,5H)-dione;
(B123) 5-[4-[(2-isopropylbenzoyl)amino]phenyl]-1,2,3,4,8,9,10,11-octahydronaphtho[1,2-b][1,4]diazepine-2,4(3H,5H)-dione;
(B124) 5-[4-[2-chloro-5-(methylthio)benzoylamino]phenyl]-1H-naphtho[1,2-b][1,4]diazepine-2,4(3H,5H)-dione;
(B125) 5-[4-[2-(methylthio)benzoylamino]phenyl]-1H-naphtho[1,2-b][1,4]diazepine-2,4(3H,5H)-dione;
(B126) 5-[4-[3-(methylthio)benzoylamino]phenyl]-1H-naphtho[1,2-b][1,4]diazepine-2,4(3H,5H)-dione;
(B127) 5-[4-[2-ethyl-6-methoxybenzoylamino]phenyl]-1H-naphtho[1,2-b][1,4]diazepine-2,4(3H,5H)-dione;
(B128) 5-[4-(3-methanesulfonybenzoylamino)phenyl]-1H-naphtho[1,2-b][1,4]diazepine-2,4(3H,5H)-dione;
(B129) 6-ethyl-1-[4-(2-iodobenzoyl)aminophenyl]-1H-1,5-benzodiazepine-2,4(3H,5H)-dione;
(B130) 5-[4-[2-ethyl-6-hydroxybenzoylamino]phenyl]-1H-naphtho[1,2-b][1,4]diazepine-2,4(3H,5H)-dione;
(B131) 5-[4-(3-methanesulfinylbenzoylamino)phenyl]-1H-naphtho[1,2-b][1,4]diazepine-2,4(3H,5H)-dione;
(B132) 5-[4-(2-chloro-5-methanesulfinylbenzoylamino)phenyl]-1H-naphtho[1,2-b][1,4]diazepine-2,4(3H,5H)-dione;
(B133) 5-[4-(2-methanesulfinylbenzoylamino)phenyl]-1H-naphtho[1,2-b][1,4]diazepine-2,4(3H,5H)-dione;
(B134) 5-[4-[[2-(4-morpholinyl)acetyl]amino]phenyl]-1,2,3,4,8,9,10,11-octahydronaphtho[1,2-b][1,4]diazepine-2,4(3H,5H)-dione hydrochloride;
(B135) 5-[4-(2-chloro-6-methoxybenzoylamino)phenyl]-1,3-dihydronaphtho[1,2-e]-1,4-diazepin-2-one;
(B136) 5-[4-[[(3-chloropyridin-2-yl)carbonyl]amino]phenyl]-1H-naphtho[1,2-b][1,4]diazepine-2,4(3H,5H)-dione;
(B137) 5-[4-(2-chloro-6-hydroxybenzoylamino)phenyl]-1,3-dihydronaphtho[1,2-e]-1,4-diazepin-2-one;
(B138) 5-[4-(3-chloro-2-methoxybenzoylamino)phenyl]-1H-naphtho[1,2-b][1,4]diazepine-2,4(3H,5H)-dione;
(B139) 5-[4-[(3-methylpyridin-2-yl)carbonylamino]phenyl]-1H-naphtho[1,2-b][1,4]diazepine-2,4(3H,5H)-dione;
(B140) 5-[4-[[(3-chloropyridin-2-yl)carbonyl]amino]phenyl]-1,2,3,4,8,9,10,11-octahydronaphtho[1,2-b][1,4]diazepine-2,4(3H,5H)-dione;
(B141) 5-[4-(3-chloro-2-hydroxybenzoylamino)phenyl]-1H-naphtho[1,2-b][1,4]diazepine-2,4(3H,5H)-dione;
(B142) 5-[4-[[(3-hydroxypyridin-2-yl)carbonyl]amino]phenyl]-1H-naphtho[1,2-b][1,4]diazepine-2,4(3H,5H)-dione;
(B143) 5-[4-[(3-vinylpyridin-2-yl)carbonylamino]phenyl]-1H-naphtho[1,2-b][1,4]diazepine-2,4(3H,5H)-dione;
(B144) 5-[4-[(3-ethylpyridin-2-yl)carbonylamino]phenyl]-1H-naphtho[1,2-b][1,4]diazepine-2,4(3H,5H)-dione;
(B145) N-[4-(2,4-dioxo-1,2,3,4-tetrahydronaphtho[1,2-b][1,4]diazepin-5-yl)phenyl]-2-nitrobenzenesulfonamide;
(B146) N-[4-(2,4-dioxo-1,2,3,4-tetrahydronaphtho[1,2-b][1,4]diazepin-5-yl)phenyl]benzenesulfonamide;
(B147) 3-bromo-N-[4-(2,4-dioxo-1,2,3,4-tetrahydronaphtho[1,2-b][1,4]diazepin-5-yl)phenyl]benzenesulfonamide;
(B148) N-[4-(2,4-dioxo-1,2,3,4-tetrahydronaphtho[1,2-b][1,4]diazepin-5-yl)phenyl]-3-methoxybenzenesulfonamide;
(B149) N-[3-(2-oxo-2,3-dihydro-1H-naphtho[1,2-e][1,4]diazepin-5-yl)phenyl]benzenesulfonamide;
(B150) N-[3-(2,4-dioxo-1,2,3,4-tetrahydronaphtho[1,2-b][1,4]diazepin-5-yl)phenyl]-2-nitrobenzenesulfonamide;
(B151) N-[4-(2,4-dioxo-1,2,3,4,8,9,10,11-octahydro-naphtho[1,2-b][1,4]diazepin-5-yl)phenyl]-2-nitrobenzenesulfonamide;
(B152) N-[4-(2,4-dioxo-1,2,3,4,8,9,10,11-octahydro-naphtho[1,2-b][1,4]diazepin-5-yl)phenyl]-N-methyl-2-nitrobenzenesulfonamide;
(B153) N-[3-(2,4-dioxo-1,2,3,4-tetrahydronaphtho[1,2-b][1,4]diazepin-5-yl)phenyl]-N-methyl-2-nitrobenzenesulfonamide;
(B154) 4-(2,4-dioxo-1,2,3,4,8,9,10,11-octahydronaphtho[1,2-b][1,4]diazepin-5-yl)-N-phenylbenzenesulfonamide;
(B155) N-[3-(2,4-dioxo-1,2,3,4-tetrahydronaphtho[1,2-b][1,4]diazepin-5-yl)phenyl]-2-naphthalenesulfonamide;
(B156) N-[3-(2,4-dioxo-1,2,3,4-tetrahydronaphtho[1,2-b][1,4]diazepin-5-yl)phenyl]-1-naphthalenesulfonamide;
(B157) N-[4-(2,4-dioxo-1,2,3,4-tetrahydronaphtho[1,2-b][1,4]diazepin-5-yl)phenyl]cyclohexanesulfonamide;
(B158) N-[4-(2,4-dioxo-1,2,3,4-tetrahydronaphtho[1,2-b][1,4]diazepin-5-yl)phenyl]-3-pyridinesulfonamide hydrochloride;
(B159) N-[4-(2,4-dioxo-1,2,3,4-tetrahydronaphtho[1,2-b][1,4]diazepin-5-yl)phenyl]-4-isopropylbenzenesulfonamide;
(B160) N-[4-(2,4-dioxo-1,2,3,4-tetrahydronaphtho[1,2-b][1,4]diazepin-5-yl)phenyl]phenylmethanesulfonamide;
(B161) N-[4-(2,4-dioxo-1,2,3,4-tetrahydronaphtho[1,2-b][1,4]diazepin-5-yl)phenyl]-2-thiophene-sulfonamide;
(B162) N-[4-(2,4-dioxo-1,2,3,4-tetrahydronaphtho[1,2-b][1,4]diazepin-5-yl)phenyl]-2-naphthalenesulfonamide;
(B163) 4-(2,4-dioxo-1,2,3,4,8,9,10,11-octahydronaphtho-[1,2-b][1,4]diazepin-5-yl)phenyl 3-bromobenzene-sulfonate;
(B164) N-benzyl-N-[4-(1-benzyl-2,4-dioxo-1,2,3,4-tetrahydronaphtho[1,2-b][1,4]diazepin-5-yl)phenyl]-2-nitrobenzenesulfonamide;
(B165) N-benzyl-N-[4-(2,4-dioxo-1,2,3,4-tetrahydronaphtho[1,2-b][1,4]diazepin-5-yl)phenyl]-2-nitrobenzenesulfonamide;
(B166) 3-bromo-N-[4-(2,4-dioxo-1,2,3,4-tetrahydronaphtho[1,2-b][1,4]diazepin-5-yl)phenyl]-N-methylbenzenesulfonamide;
(B167) N-[4-(2,4-dioxo-1,2,3,4-tetrahydronaphtho[1,2-b][1,4]diazepin-5-yl)phenyl]-N-methyl-2-nitrobenzenesulfonamide;
(B168) N-[4-(2,4-dioxo-1,2,3,4-tetrahydronaphtho[1,2-b][1,4]diazepin-5-yl)phenyl]-N-(2-hydroxyethyl)-2-nitrobenzenesulfonamide;
(B169) N-[4-(7-chloro-2,4-dioxo-2,3,4,5-tetrahydro-1H-benzo[b][1,4]diazepin-1-yl)phenyl]benzenesulfonamide;
(B170) N-[4-(7-bromo-2,4-dioxo-2,3,4,5-tetrahydro-1H-benzo[b][1,4]diazepin-1-yl)phenyl]benzenesulfonamide;
(B171) N-[4-[(2,4-dioxo-7-(trifluoromethyl)-2,3,4,5-tetrahydro-1H-benzo[b][1,4]diazepin-1-yl)]phenyl]benzenesulfonamide;
(B172) N-[4-(2,4-dioxo-2,3,4,5-tetrahydro-1H-benzo[b][1,4]diazepin-1-yl)phenyl]benzenesulfonamide;
(B173) 1-(2-chlorophenyl)-N-[4-(2,4-dioxo-1,2,3,4-tetrahydronaphtho[1,2-b][1,4]diazepin-5-yl)phenyl]methanesulfonamide;
(B174) 1-(3-bromophenyl)-N-[4-(2,4-dioxo-1,2,3,4-tetrahydronaphtho[1,2-b][1,4]diazepin-5-yl)phenyl]methanesulfonamide;
(B175) N-[4-(2,4-dioxo-1,2,3,4-tetrahydronaphtho[1,2-b][1,4]diazepin-5-yl)phenyl]-2-trifluoromethylbenzenesulfonamide;
(B176) N-[4-(7-bromo-6-methyl-2,4-dioxo-2,3,4,5-tetrahydro-1H-benzo[b][1,4]diazepin-1-yl)phenyl]benzenesulfonamide;
(B177) 1-(2-chlorophenyl)-N-[4-(2,4-dioxo-1,2,3,4,8,9,10,11-octahydronaphtho[1,2-b][1,4]diazepin-5-yl)phenyl]methanesulfonamide;
(B178) 3-bromo-N-[4-(2,4-dioxo-1,2,3,4,8,9,10,11-octahydronaphtho[1,2-b][1,4]diazepin-5-yl)phenyl]benzenesulfonamide;
(B179) N-[4-(2,4-dioxo-1,2,3,4,8,9,10,11-octahydronaphtho[1,2-b][1,4]diazepin-5-yl)phenyl]-3-methoxybenzenesulfonamide;
(B180) 1-(2-bromophenyl)-N-[4-(2,4-dioxo-1,2,3,4-tetrahydronaphtho[1,2-b][1,4]diazepin-5-yl)phenyl]methanesulfonamide;
(B181) N-[4-(2,4-dioxo-1,2,3,4-tetrahydronaphtho[1,2-b][1,4]diazepin-5-yl)phenyl]-1-(2-methylphenyl)methanesulfonamide;
(B182) N-[4-(2,4-dioxo-1,2,3,4-tetrahydronaphtho[1,2-b][1,4]diazepin-5-yl)phenyl]-1-(2-nitrophenyl)methanesulfonamide;
(B183) N-[4-(2,4-dioxo-1,2,3,4-tetrahydronaphtho[1,2-b][1,4]diazepin-5-yl)phenyl]-2-phenylethanesulfonamide;
(B184) 1-(2,3-dichlorophenyl)-N-[4-(2,4-dioxo-1,2,3,4,8,9,10,11-octahydronaphtho[1,2-b][1,4]diazepin-5-yl)phenyl]methanesulfonamide;
(B185) 1-(2-chlorophenyl)-N-[4-(2,4-dioxo-7-methoxy-1H-benzo[1,2-b][1,4]diazepin-1-yl)phenyl]methanesulfonamide;
(B186) 1-(2-chlorophenyl)-N-[4-(2,4-dioxo-7-hydroxy-1H-benzo[1,2-b][1,4]diazepin-1-yl)phenyl]methanesulfonamide;
(B187) 1-(4-chlorophenyl)-N-[4-(2,4-dioxo-1,2,3,4-tetrahydronaphtho[1,2-b][1,4]diazepin-5-yl)phenyl]methanesulfonamide;
(B188) 1-(2-chlorophenyl)-N-[4-(2,4-dioxo-1,2,3,4-tetrahydronaphtho[1,2-b][1,4]diazepin-5-yl)benzyl]methanesulfonamide;
(B189) 1-(2-chlorophenyl)-N-[4-(2,4-dioxo-1,2,3,4-tetrahydronaphtho[1,2-b][1,4]diazepin-5-yl)-2-methoxyphenyl]methanesulfonamide;
(B190) 1-(2-chlorophenyl)-N-[4-(2,4-dioxo-1,2,3,4-tetrahydronaphtho[1,2-b][1,4]diazepin-5-yl)-2-hydroxyphenyl]methanesulfonamide;
(B191) 1-(2,6-dichlorophenyl)-N-[4-(2,4-dioxo-1,2,3,4,8,9,10,11-octahydronaphtho[1,2-b][1,4]diazepin-5-yl)phenyl]methanesulfonamide;
(B192) 1-(2-chlorophenyl)-N-[4-(2,4-dioxo-6-methyl-1H-benzo[1,2-b][1,4]diazepin-1-yl)phenyl]methanesulfonamide;
(B193) 1-(2-chlorophenyl)-N-[3-(2,4-dioxy-1,2,3,4-tetrahydronaphtho[1,2-b][1,4]diazepin-5-yl) propyl]methanesulfonamide;
(B194) 1-(2-chlorophenyl)-N-[2-(2,4-dioxo-1,2,3,4-tetrahydronaphtho[1,2-b][1,4]diazepin-5-yl)ethyl]methanesulfonamide;
(B195) N-[4-(2,4-dioxo-1,2,3,4,8,9,10,11-octahydronaphtho[1,2-b][1,4]diazepin-5-yl)phenyl]-1-(2-iodophenyl)methanesulfonamide;
(B196) 1-(2-chlorophenyl)-N-[4-(2,4-dioxo-1,2,3,4,8,9,10,11-octahydronaphtho[1,2-b][1,4]diazepin-5-yl)phenyl]-N-methylmethanesulfonamide;
(B197) 1-(2-chlorophenyl)-N-[4-(2-oxo-2,3-dihydro-1H-naphtho[1,2-e][1,4]diazepin-5-yl)phenyl]methanesulfonamide;
(B198) 1-[(2-trifluoromethyl)phenyl]-N-[4-(2,4-dioxo-1,2,3,4-tetrahydronaphtho[1,2-b][1,4]diazepin-5-yl)phenyl]phenyl-N-methylmethanesulfonamide;
(B199) 1-(2-ethylphenyl)-N-[4-(2,4-dioxo-1,2,3,4-tetrahydronaphtho[1,2-b][1,4]diazepin-5-yl)phenyl]phenyl-N-methylmethanesulfonamide;
(B200) 1-(2,3-dimethylphenyl)-N-[4-(2,4-dioxo-1,2,3,4-tetrahydronaphtho[1,2-b][1,4]diazepin-5-yl)phenyl]phenyl-N-methylmethanesulfonamide;
(B201) 2-(2-chlorophenyl)-N-[4-(2,4-dioxo-1,2,3,4-tetrahydronaphtho[1,2-b][1,4]diazepin-5-yl)phenyl]phenyl-N-methylethanesulfonamide;
(B202) 1-(2-nitrophenyl)-N-[4-(2,4-dioxo-1,2,3,4-tetrahydronaphtho[1,2-b][1,4]diazepin-5-yl)phenyl]phenyl-N-methylmethanesulfonamide;
(B203) 1-(2-aminophenyl)-N-[4-(2,4-dioxo-1,2,3,4-tetrahydronaphtho[1,2-b][1,4]diazepin-5-yl)phenyl]phenyl-N-methylmethanesulfonamide;
(B204) 1-(2-dimethylaminophenyl)-N-[4-(2,4-dioxo-1,2,3,4-tetrahydronaphtho[1,2-b][1,4]diazepin-5-yl)phenyl]phenyl-N-methylmethanesulfonamide;
(B205) 5-[4-[(pyridin-4-yl)carbonylamino]phenyl]-1H-naphtho[1,2-b][1,4]diazepine-2,4(3H,5H)-dione hydrochloride;
(B206) 5-[4-[2-[(pyridin-3-yl)oxy]acetylamino]phenyl]-1H-naphtho[1,2-b][1,4]diazepine-2,4(3H,5H)-dione hydrochloride;
(B207) 5-[4-[(pyridin-3-yl)carbonylamino]phenyl]-1H-naphtho[1,2-b][1,4]diazepine-2,4(3H,5H)-dione hydrochloride;
(B208) 5-[4-[(2-methylpyridin-3-yl)carbonylamino]phenyl]-1H-naphtho[1,2-b][1,4]diazepine-2,4(3H,5H)-dione hydrochloride;
(B209) 5-[4-[(2-chloropyridin-3-yl)carbonylamino]phenyl]-1H-naphtho[1,2-b][1,4]diazepine-2,4(3H,5H)-dione;
(B210) 5-[4-[2-[(pyridin-2-yl)oxy]acetylamino]phenyl]-1H-naphtho[1,2-b][1,4]diazepine-2,4(3H,5H)-dione;
(B211) 5-[4-[[4-(trifluoromethyl)pyridin-3-yl]carbonylamino]phenyl]-1H-naphtho[1,2-b][1,4]diazepine-2,4(3H,5H)-dione;
(B212) 5-[4-[(2-chloropyridin-3-yl)carbonylamino]phenyl]-1H-[1,4]diazepino[2,3-f]isoquinoline-2,4(3H,5H)-dione;
(B213) 5-[4-[(2-chloropyridin-3-yl)carbonylamino]phenyl]-8,9,10,11-tetrahydro-1H-[1,4]diazepino[2,3-f]isoquinoline-2,4(3H,5H)-dione; and
(B214) 5-[4-[(2-isopropylbenzoyl)amino]phenyl]-1H-naphtho[1,2-b][1,4]diazepine-2,4(3H,5H)-dione.

18. The method according to claim 12, wherein the nociceptive pain is inflammatory pain.

19. The method according to claim 18, wherein the inflammatory pain is pain due to inflammatory bowel disease.

20. The method according to claim 13, wherein the visceral pain is visceral pain caused by the intestinal tract.

21. The method according to claim 20, wherein the visceral pain caused by the intestinal tract is visceral pain caused by inflammatory bowel disease.

22. The method according to claim 13, wherein the visceral pain is pain after inflammation is relieved or healed.
